SEQUENCE LISTING (4 sequences)

SEQ ID NO: 1 - protein, 95 aa, Homo sapiens
VSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTSS

SEQ ID NO: 2 - protein, 105 aa, Homo sapiens
TITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQ

SEQ ID NO: 3 - protein, 96 aa, Homo sapiens
VSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 4 - protein, 105 aa, Homo sapiens; identical to SEQ ID NO: 2

(19) United States
(12) Patent Application Publication
(10) Pub. No.: US 2025/0221970 A1
(43) Pub. Date: Jul. 10, 2025

(54) Combinations of a B-RAF Inhibitor and an Anti-EGFR Antibody for the Treatment of Cancer

(71) Applicants: MapKure, LLC, Wilmington, DE (US); Amgen Inc., Thousand Oaks, CA (US)

(72) Inventor: Lusong Luo, San Mateo, CA (US)

(73) Assignees: MapKure, LLC, Wilmington, DE (US); Amgen Inc., Thousand Oaks, CA (US)

(21) Appl. No.: 19/089,489

(22) Filed: Mar. 25, 2025

Related U.S. Application Data
(63) Continuation of application No. PCT/US23/75047, filed on Sep. 25, 2023.
(60) Provisional application No. 63/409,862, filed on Sep. 26, 2022.
(60) Provisional application No. 63/515,217, filed on Jul. 24, 2023.

(51) Int. Cl. A61K 31/4375; A61K 39/00; A61K 39/395

(52) U.S. Cl. CPC A61K 31/4375; A61K 39/39558; A61K 2039/545

(57) ABSTRACT

Provided here is a combination comprising
(i) Compound A having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or the structure of formula (I):

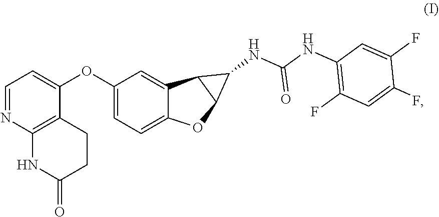 [SMILES: O=C1CCc2c(Oc3ccc4c(c3)[C@H]3[C@H](NC(=O)Nc5cc(F)c(F)cc5F)[C@H]3O4)ccnc2N1]

or a pharmaceutically acceptable salt, tautomer, stereoisomer, enantiomer, isotopologue, solvate, or prodrug thereof; and an anti-EGFR antibody, e.g., panitumumab; compositions comprising the same; and methods of using such combinations and compositions in the treatment of cancer, e.g., colorectal cancer, pancreatic cancer, and non-small cell lung cancer.

### CROSS-REFERENCE TO RELATED APPLICATIONS

This application is a continuation of International Application No. PCT/US23/75047, filed on Sep. 25, 2023, which claims priority to U.S. Provisional Application No. 63/409,862, filed on Sep. 26, 2022 and U.S. Provisional Application No. 63/515,217, filed on Jul. 24, 2023, the disclosures of each of which are hereby incorporated by reference in their entireties.

### FIELD

Provided herein is a novel combination comprising a B-Raf inhibitor, particularly 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea or a pharmaceutically acceptable salt thereof, and an anti-EGFR antibody; pharmaceutical compositions comprising the same and methods of using such combinations and compositions in the treatment of conditions in which the inhibition of B-Raf, KRAS, NRAS, and/or EGFR is beneficial, e.g., cancer.

### SEQUENCE LISTING

The instant application contains a Sequence Listing, which has been submitted electronically in XML format and is hereby incorporated by reference in its entirety. Said XML copy, created on Sep. 25, 2023, is named “01368-0003-00PCT.xml” and is 4,887 bytes in size.

### BACKGROUND

Effective treatment of hyperproliferative disorders including cancer is a continuing goal in the oncology field. Generally, cancer results from the deregulation of the normal processes that control cell division, differentiation and apoptotic cell death and is characterized by the proliferation of malignant cells, which have the potential for unlimited growth, local expansion and systemic metastasis. Deregulation of normal processes includes abnormalities in signal transduction pathways and response to factors that differ from those found in normal cells.

An important large family of enzymes is the protein kinase enzyme family. Currently, there are about 500 different known protein kinases. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase is regulated by one or more kinases. Consequently, aberrant or inappropriate protein kinase activity can contribute to the rise of disease states associated with such aberrant kinase activity including benign and malignant proliferative disorders as well as diseases resulting from inappropriate activation of the immune and nervous systems. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The protein serine/threonine kinases (PSTK) include cyclic AMP- and cyclic GMP-dependent protein kinases, calcium and phospholipid dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also in progress to identify modulators of tyrosine kinases.

Receptor tyrosine kinases (RTKs) catalyze phosphorylation of certain tyrosyl amino acid residues in various proteins, including themselves, which govern cell growth, proliferation and differentiation.

Downstream of the several RTKs lie several signaling pathways; among them is the Ras-Raf-MEK-ERK kinase pathway. It is currently understood that activation of Ras GTPase proteins in response to growth factors, hormones, cytokines, etc. stimulates phosphorylation and activation of Raf kinases. These kinases then phosphorylate and activate the intracellular protein kinases MEK1 and MEK2, which in turn phosphorylate and activate other protein kinases, ERK1 and 2. This signaling pathway, also known as the mitogen-activated protein kinase (MAPK) pathway or cytoplasmic cascade, mediates cellular responses to growth signals. The ultimate function of this is to link receptor activity at the cell membrane with modification of cytoplasmic or nuclear targets that govern cell proliferation, differentiation, and survival.

The constitutive activation of this pathway is sufficient to induce cellular transformation. Dysregulated activation of the MAP kinase pathway due to aberrant receptor tyrosine kinase activation, Ras mutations or Raf mutations has frequently been found in human cancers, and represents a major factor determining abnormal growth control. In human malignances, Ras mutations are common, having been identified in about 30% of cancers. The Ras family of GTPase proteins (proteins which convert guanosine triphosphate to guanosine diphosphate) relay signals from activated growth factor receptors to downstream intracellular partners. Prominent among the targets recruited by active membrane-bound Ras are the Raf family of serine/threonine protein kinases. The Raf family is composed of three related kinases (A-, B- and C-Raf) that act as downstream effectors of Ras. Ras-mediated Raf activation in turn triggers activation of MEK1 and MEK2 (MAP/ERK kinases 1 and 2) which in turn phosphorylate ERK1 and ERK2 (extracellular signal-regulated kinases 1 and 2) on the tyrosine-185 and threonine-183. Activated ERK1 and ERK2 translocate and accumulate in the nucleus, where they can phosphorylate a variety of substrates, including transcription factors that control cellular growth and survival.

Mutations in various Ras GTPases and the B-Raf kinase have been identified that can lead to sustained and constitutive activation of the MAPK pathway, ultimately resulting in increased cell division and survival. As a consequence of this, these mutations have been strongly linked with the establishment, development, and progression of a wide range of human cancers. The biological role of the Raf kinases, and specifically that of B-Raf, in signal transduction is described in Davies, H., et al., Nature (2002) 9:1-6; Garnett, M. J. & Marais, R., Cancer Cell (2004) 6:313-319; Zebisch, A. & Troppmair, J., Cell. Mol. Life Sci. (2006) 63:1314-1330; Midgley, R. S. & Kerr, D. J., Crit. Rev. Onc/Hematol. (2002) 44:109-120; Smith, R. A., et al., Curr. Top. Med. Chem. (2006) 6:1071-1089; and Downward, J., Nat. Rev. Cancer (2003) 3:11-22.

Naturally occurring mutations of the B-Raf kinase that activate MAPK pathway signaling have been found in a large percentage of human melanomas (Davies (2002) supra) and thyroid cancers (Cohen et al J. Nat. Cancer Inst. (2003) 95 (8) 625-627 and Kimura et al Cancer Res. (2003) 63 (7) 1454-1457), as well as at lower, but still significant, frequencies in the following:

Barret's adenocarcinoma (Garnett et al., Cancer Cell (2004) 6 313-319 and Sommerer et al Oncogene (2004) 23 (2) 554-558), billiary tract carcinomas (Zebisch et al., Cell. Mol. Life Sci. (2006) 63 1314-1330), breast cancer (Davies (2002) supra), cervical cancer (Moreno-Bueno et al Clin. Cancer Res. (2006) 12 (12) 3865-3866), cholangiocarcinoma (Tannapfel et al Gut (2003) 52(5) 706-712), central nervous system tumors including primary CNS tumors such as glioblastomas, astrocytomas and ependymomas (Knobbe et al Acta Neuropathol. (Berl.) (2004) 108(6) 467-470, Davies (2002) supra, and Garnett et al., Cancer Cell (2004) supra) and secondary CNS tumors (i.e., metastases to the central nervous system of tumors originating outside of the central nervous system), colorectal cancer, including large intestinal colon carcinoma (Yuen et al Cancer Res. (2002) 62(22) 6451-6455, Davies (2002) supra and Zebisch et al., Cell. Mol. Life Sci. (2006), gastric cancer (Lee et al Oncogene (2003) 22(44) 6942-6945), carcinoma of the head and neck including squamous cell carcinoma of the head and neck (Cohen et al J. Nat. Cancer Inst. (2003) 95(8) 625-627 and Weber et al Oncogene (2003) 22(30) 4757-4759), hematologic cancers including leukemias (Garnett et al., Cancer Cell (2004) supra, particularly acute lymphoblastic leukemia (Garnett et al., Cancer Cell (2004) supra and Gustafsson et al Leukemia (2005) 19(2) 310-312), acute myelogenous leukemia (AML) (Lee et al Leukemia (2004) 18(1) 170-172, and Christiansen et al Leukemia (2005) 19(12) 2232-2240), myelodysplastic syndromes (Christiansen et al Leukemia (2005) supra) and chronic myelogenous leukemia (Mizuchi et al Biochem. Biophys. Res. Commun. (2005) 326(3) 645-651); Hodgkin's lymphoma (Figl et al Arch. Dermatol. (2007) 143(4) 495-499), non-Hodgkin's lymphoma (Lee et al Br. J. Cancer (2003) 89(10) 1958-1960), megakaryoblastic leukemia (Eychene et al Oncogene (1995) 10(6) 1159-1165) and multiple myeloma (Ng et al Br. J. Haematol. (2003) 123(4) 637-645), hepatocellular carcinoma (Garnett et al., Cancer Cell (2004), lung cancer (Brose et al Cancer Res. (2002) 62(23) 6997-7000, Cohen et al J. Nat. Cancer Inst. (2003) supra and Davies (2002) supra), including small cell lung cancer (Pardo et al EMBO J (2006) 25(13) 3078-3088) and non-small cell lung cancer (Davies (2002) supra), ovarian cancer (Russell & McCluggage J. Pathol. (2004) 203(2) 617-619 and Davies (2002) supr), endometrial cancer (Garnett et al., Cancer Cell (2004) supra, and Moreno-Bueno et al Clin. Cancer Res. (2006) supra), pancreatic cancer (Ishimura et al Cancer Lett. (2003) 199(2) 169-173), pituitary adenoma (De Martino et al J. Endocrinol. Invest. (2007) 30(1) RC1-3), prostate cancer (Cho et al Int. J. Cancer (2006) 119(8) 1858-1862), renal cancer (Nagy et al Int. J. Cancer (2003) 106(6) 980-981), sarcoma (Davies (2002) supra), and skin cancers (Rodriguez-Viciana et al Science (2006) 311(5765) 1287-1290 and Davies (2002) supra). Overexpression of c-Raf has been linked to AML (Zebisch et al., Cancer Res. (2006) 66(7) 3401-3408, and Zebisch (Cell. Mol. Life Sci. (2006)) and erythroleukemia (Zebisch et al., Cell. Mol. Life Sci. (2006).

Epidermal Growth Factor Receptor (EGFR) is the cell-surface receptor for members of epidermal growth factor family and is activated by binding to specific ligands, including epidermal growth factor. Upon activation, EGFR undergoes a transition from an inactive monomer form to an active homodimer (Yarden et al Biochemistry, 26 (5) 1443-1451). The homodimer stimulates intracellular protein tyrosine kinase activity. As a result, several tyrosine residues in the C-terminal domain of EGFR are phosphorylated (Downward et al, Nature 311 (5985) 483-485). This phosphorylation elicits downstream activation and initiates several signal transduction cascades, principally the MAPK, Akt and JNK pathways, eventually causing DNA synthesis and cell proliferation (Oda et al Mol. Syst. Biol. 1(1)).

Overexpression of Epidermal Growth Factor Receptor (EGFR) has been associated with a number of cancers, including lung cancer, anal cancer and glioblastoma (Walker et al, Hum. Pathol. 40(11) 1517-1527). Inhibition of EGFR is an effective treatment for certain cancers; however, many patients develop resistance (Jackman et al Clin. Cancer. Res. 15 (16) 5267-5273).

Panitumumab (Vectibix) is a fully human monoclonal antibody that binds specifically to and antagonizes EGFR. Panitumumab is approved by the U.S. Food and Drug Administration for the treatment of wild-type RAS metastatic colorectal cancer (mCRC) in combination with FOLFOX for first-line treatment and as a monotherapy following disease progression after prior treatment with fluoropyrimidine, oxaliplatin, and irinotecan-containing chemotherapy. It is marketed by Amgen Inc. under the brand name of Vectibix. Other anti-EGFR antibodies include, but are not limited to, cetuximab, zalutumumab, nimotuzumab, and matuzumab.

Though there have been many recent advances in the treatment of cancer, there remains a need for more effective and/or alternative treatments for individuals (e.g., individuals with B-Raf, KRAS, or NRAS mutations) suffering from the effects of cancer.

Citation or identification of any reference in this section is not to be construed as an admission that the reference is prior art to the present application.

### SUMMARY

Provided here is a combination of Compound A having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or the structure of formula (I):

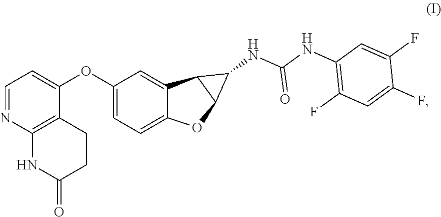 [SMILES: O=C1CCc2c(Oc3ccc4c(c3)[C@H]3[C@H](NC(=O)Nc5cc(F)c(F)cc5F)[C@H]3O4)ccnc2N1]

or a pharmaceutically acceptable salt, tautomer, stereoisomer, enantiomer, isotopologue, solvate, or prodrug thereof, and an anti-EGFR antibody. In some embodiments, the anti-EGFR antibody is panitumumab, cetuximab, zalutumumab, nimotuzumab, or matuzumab or an antigen binding fragment thereof. In one embodiment, the anti-EGFR antibody is panitumumab or an antigen binding fragment thereof. In one embodiment, the combination comprises an anti-EGFR antibody. In one embodiment, the anti-EGFR antibody is panitumumab.

The combination provided herein is useful in the treatment of cancer. In one embodiment, provided herein is a method of treating cancer in a mammal in need thereof (e.g., a human) which comprises administering a therapeutically effective amount of a combination provided herein. In one embodiment, Compound A is administered at about 5 mg, about 10 mg, about 15 mg, about 20 mg, about 25 mg, about 30 mg, or about 40 mg. In one embodiment, Compound A is administered once a day. In one embodiment, panitumumab is administered in an amount of about 6 mg/kg as an intravenous infusion over about 60 minutes every two weeks.

In one embodiment, the cancer is colorectal cancer, pancreatic cancer, or non-small cell lung cancer. In one embodiment, the cancer is colorectal cancer. In one embodiment, the cancer is metastatic colorectal cancer. In one embodiment, the cancer is BRAF mutant metastatic colorectal cancer. In one embodiment, the cancer is BRAF V600E mutant metastatic colorectal cancer. In one embodiment, the cancer is KRAS mutant colorectal cancer. In one embodiment, the cancer is KRAS G12C mutant colorectal cancer. In one embodiment, the cancer is KRAS G12D mutant colorectal cancer. In one embodiment, the cancer is KRAS G12V mutant colorectal cancer. In one embodiment, the cancer is Trp53 mutant colorectal cancer. In one embodiment, the cancer is NRAS mutant colorectal cancer. In one embodiment, the cancer is KRAS/NRAS mutant colorectal cancer.

In one embodiment, the cancer is pancreatic cancer. In one embodiment, the cancer is pancreatic ductal adenocarcinoma (PDAC). In one embodiment, the cancer is BRAF mutant pancreatic cancer. In one embodiment, the cancer is BRAF V600E mutant pancreatic cancer. In one embodiment, the cancer is KRAS mutant pancreatic cancer. In one embodiment, the cancer is KRAS G12C mutant pancreatic cancer. In one embodiment, the cancer is KRAS G12D mutant pancreatic cancer. In one embodiment, the cancer is KRAS G12V mutant pancreatic cancer. In one embodiment, the cancer is Trp53 mutant pancreatic cancer. In one embodiment, the cancer is NRAS mutant pancreatic cancer.

In one embodiment, the cancer is non-small cell lung cancer. In one embodiment, the cancer is BRAF mutant non-small cell lung cancer. In one embodiment, the cancer is BRAF V600E mutant non-small cell lung cancer. In one embodiment, the cancer is KRAS mutant non-small cell lung cancer. In one embodiment, the cancer is KRAS G12C mutant non-small cell lung cancer. In one embodiment, the cancer is KRAS G12D mutant non-small cell lung cancer. In one embodiment, the cancer is KRAS G12V mutant non-small cell lung cancer. In one embodiment, the cancer is Trp53 mutant non-small cell lung cancer. In one embodiment, the cancer is NRAS mutant non-small cell lung cancer.

In one embodiment, the method as described herein provides a plasma Compound A AUC₈ₕ between about 2,128 ng*h/ml and about 3,192 ng*h/ml in the subject. In one embodiment, the method as described herein provides a plasma Compound A AUC₈ₕ between about 4,576 ng*h/ml and about 6,864 ng*h/ml in the subject. In one embodiment, the method as described herein provides a plasma Compound A AUC₈ₕ between about 7,944 ng*h/ml and about 11,916 ng*h/ml in the subject. In one embodiment, the method as described herein provides a plasma Compound A AUC₈ₕ between about 9,840 ng*h/ml and about 14,760 ng*h/ml in the subject. In one embodiment, the method as described herein provides a plasma Compound A AUC₈ₕ between about 12,640 ng*h/ml and about 18,960 ng*h/ml in the subject. In one embodiment, the method as described herein provides a plasma Compound A AUC₈ₕ between about 30,000 ng*h/ml and about 45,000 ng*h/ml in the subject.

In one embodiment, the subject achieves a stable disease, a partial response, or a complete response. In one embodiment, the subject does not experience a progressive disease.

In one embodiment, provided herein is use of a combination of Compound A or a pharmaceutically acceptable salt, tautomer, stereoisomer, enantiomer, isotopologue, solvate, or prodrug thereof and panitumumab in the manufacture of a medicament for treating a cancer in a subject in need thereof. In one embodiment, provided herein is use of Compound A or a pharmaceutically acceptable salt, tautomer, stereoisomer, enantiomer, isotopologue, solvate, or prodrug thereof in the manufacture of a medicament for treating a cancer in a subject in need thereof, where the medicament is suitable for being administered with panitumumab. In one embodiment, provided here is use of panitumumab in the manufacture of a medicament for treating a cancer in a subject in need thereof, where the medicament is suitable for being administered with Compound A.

### BRIEF DESCRIPTION OF DRAWINGS

FIG. 1 describes the safety run-in and dose escalation plan.

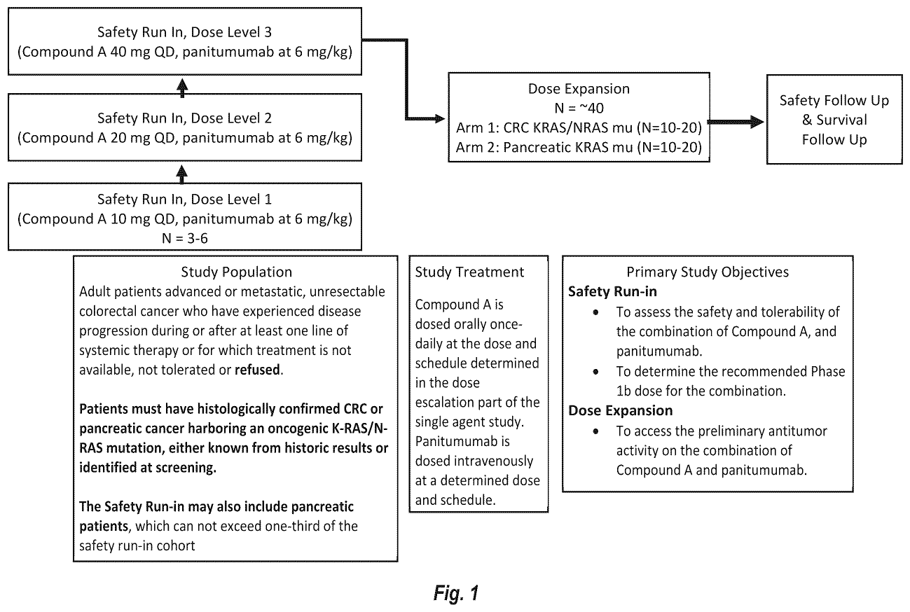

FIG. 2 describes the study design: Part 1 (Dose Finding) and Part 2 (Dose Expansion).

FIG. 3 describes the study periods.

### DETAILED DESCRIPTION

### Definitions

As used herein, “Compound A” refers to the compound having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or the structure of formula (I):

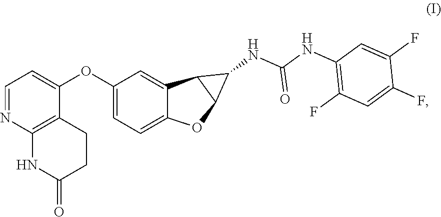 [SMILES: O=C1CCc2c(Oc3ccc4c(c3)[C@H]3[C@H](NC(=O)Nc5cc(F)c(F)cc5F)[C@H]3O4)ccnc2N1]

or a pharmaceutically acceptable salt, tautomer, stereoisomer, enantiomer, isotopologue, solvate, or prodrug thereof. Compound A is disclosed and claimed, along with pharmaceutically acceptable salts thereof, and also as solvates thereof, as being useful as an inhibitor of BRAF activity, particularly in treatment of cancer, in WO2014206343 and WO2020151756, the entire disclosures of which are incorporated herein by reference. Compound A is Compound 1.49 in WO2014206343 and Compound 1 in WO2020151756. Compound A can be prepared as described in WO2014206343 and WO2020151756. In one embodiment, Compound A is a hydrate. Unless specifically stated, “Compound A” as used herein refers to Compound A or a pharmaceutically acceptable salt, tautomer, stereoisomer, enantiomer, isotopologue, solvate, or prodrug thereof.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. The term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology 3^{rd} unology, Ed., W.H. Freeman & Co., New York, 1997.

More particularly, “antibody” refers to a polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the V_{L}, V_{H}, C_{L} and C_{H}, domains; a F(ab′)₂ fragment, a bivalent fragment comprising two Fab fragments linked by a disulfide bridge at the hinge region; a F_{d} fragment consisting of the V_{H} and C_{H}, domains; a F, fragment consisting of the V_{L} and V_{H} domains of a single arm of an antibody, a dAb fragment (Ward et al. (1989) Nature 341:544-546), which consists of a V_{H} domain; and an isolated complementarity determining region (CDR). Furthermore, although the two domains of the Fᵥ fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fᵥ (scFᵥ)). Bird et al. (1988) Science 242:423-426 and Huston et al. (1988) Proc. Natl. Acad Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

“Antibody fragments” or “antigen binding fragments” include proteolytic antibody fragments (such as F(ab′)₂ fragments, Fab′ fragments, Fab′-SH fragments and Fab fragments as are known in the art), recombinant antibody fragments (such as sFᵥ fragments, dsFᵥ fragments, bispecific sFᵥ fragments, bispecific dsFᵥ fragments, F(ab)′₂ fragments, single chain Fv proteins (“scFᵥ”), disulfide stabilized F, proteins (“dsFᵥ”), diabodies, and triabodies (as are known in the art), and camelid antibodies (see, for example, U.S. Pat. Nos. 6,015,695; 6,005,079; 5,874,541; 5,840,526; 5,800,988; and 5,759,808). An scFᵥ protein is a fusion protein in which a light chain variable region of an immunoglobulin and a heavy chain variable region of an immunoglobulin are bound by a linker, while in dsFᵥs, the chains have been mutated to introduce a disulfide bond to stabilize the association of the chains.

The term “anti-EGFR antibody,” as used herein, generally refers to an antibody or an antigen-binding fragment thereof that specifically or preferentially binds an EGFR. In some cases, and anti-EGFR antibody may bind to a mutated form of EGFR (e.g., EGFR variant III (also known as EGFRvIII), which is the most common extracellular domain mutation of EGFR; this mutation leads to a deletion of exons 2-7 of the EGFR gene and renders the mutant receptor incapable of binding any known ligand). For example, an anti-EGFR antibody may be panitumumab, cetuximab, zalutumumab, nimotuzumab, or matuzumab thereof.

Panitumumab (Vectibix) is a recombinant human IgG2 monoclonal antibody that binds specifically to the human EGFR that can be made according to the procedure described in US Patent Publication No. 2015/0152184 which is incorporated herein by reference in its entirety. The sequence of the heavy and light chains for panitumumab are known in the art and can also be found in public databases, e.g., Inxight Drugs developed by The National Center for Advancing Translational Sciences (NCATS). In one embodiment, panitumumab is the generic, compendial, nonproprietary, or official FDA name for the product marketed as Vectibix by Amgen and a product that is interchangeable with or equivalent to the product marketed as Vectibix.

In one embodiment, panitumumab is a recombinant human IgG2 monoclonal antibody that binds specifically to the human EGFR. It comprises of one heavy chain variable region and a light chain variable region, and can be made according the procedure described in U.S. Pat. No. 6,235,883. The sequence of the heavy chain and its matching light chain for panitumumab as disclosed in U.S. Pat. No. 6,235,883 are listed below:

Heavy chain 1
(SEQ ID NO: 1)
VSGGSVSSGD YYWTWIRQSP GKGLEWIGHI YYSGNTNYNP
SLKSRLTISI DTSKTQFSLK LSSVTAADTA IYYCVRDRVT
GAFDIWGQGT MVTSS
Light chain 1
(SEQ ID NO: 2)
TITCQASQDI SNYLNWYQQK PGKAPKLLIY DASNLETGVP
SRFSGSGSGT DFTFTISSLQ PEDIATYFCQ HFDHLPLAFG
VAAPSVFIFP PSDEQ

The above heavy chain 1 and light chain 1 correspond to sequences 37 and 38 in U.S. Pat. No. 6,235,883 which is incorporated herein by reference in its entirety.

Heavy chain 2
(SEQ ID NO: 3)
VSGGSVSSGDYYWTWIRQSPGKGLEWIGHIYYSGNTNYNPSLKSRLTIS
IDTSKTQFSLKLSSVTAADTAIYYCVRDRVTGAFDIWGQGTMVTVSS
Light chain 2
(SEQ ID NO: 4)
TITCQASQDISNYLNWYQQKPGKAPKLLIYDASNLETGVPSRFSGSGSG
TDFTFTISSLQPEDIATYFCQHFDHLPLAFGGGTKVEIKRTVAAPSVFI
FPPSDEQ

The above heavy chain 2 and light chain 2 correspond to sequences 76 and 54 in U.S. Pat. No. 7,807,798 which is incorporated herein by reference in its entirety.

In one embodiment, panitumumab is an isolated human antibody as disclosed in U.S. Pat. No. 7,807,798 that binds to human epidermal growth factor receptor (EGF-r). In one embodiment, panitumumab is an isolated human antibody comprising a heavy chain immunoglobulin molecule comprising:
a) a CDR1 comprising amino acids 8 to 15 of SEQ ID NO: 3,
b) a CDR2 comprising amino acids 29 to 45 of SEQ ID NO: 3; and
c) a CDR3 comprising amino acids 77 to 85 of SEQ ID NO: 3;
and comprising a light chain immunoglobulin molecule comprising.
d) a CDR1 comprising amino acids 5 to 15 of SEQ ID NO: 4;
e) a CDR2 comprising amino acids 31 to 37 of SEQ ID NO. 4; and
f) a CDR3 comprising amino acids 70 to 78 of SEQ ID NO: 4:

The sequences of cetuximab, zalutumumab, nimotuzumab, and matuzumab are also known in the art and can also be found in public databases, e.g., Inxight Drugs developed by The National Center for Advancing Translational Sciences (NCATS). Cetuximab, zalutumumab, nimotuzumab, and matuzumab can readily prepared by the common knowledge in the art. In one embodiment, cetuximab is an anti-epidermal growth factor receptor monoclonal antibody Mab C225 as defined in U.S. Pat. No. 7,960,516 B2 which is incorporated herein by reference in its entirety. In one embodiment, cetuximab is an anti-EGFR antibody described in U.S. Pat. No. 4,943,533 and WO 96/40210. In one embodiment, zalutuzumab (Humax-EGFR) is an anti-EGFR antibody described in WO 02/100348 and WO 2004/056847. In one embodiment, nimotuzumab (TheraCIM hR3) is an anti-EGFR antibody described in U.S. Pat. Nos. 5,891,996 and 6,506,883. In one embodiment, matuzumab (EMD72000) is an anti-EGFR antibody described in WO 02/66058. The disclosures of these references are incorporated herein by reference in their entirety.

In one embodiment, a solid form of Compound A is used for the treatment provided herein. In one embodiment, a crystal form of Compound A is used for the treatment provided herein. In one embodiment, an amorphous form of Compound A is used for the treatment provided herein. In one embodiment, the free base of Compound A is used for the treatment provided herein. In one embodiment, a HCl salt of Compound A is used for the treatment provided herein. In one embodiment, a solid form of Compound A described in WO2020151756 is used for the treatment provided herein. In one embodiment, a solid form of Compound A described in WO2020151756 is used for the treatment provided herein. In one embodiment, Forms A, A*, A**, B, C, D, E, F, G, H, I, J, or K of Compound A as described in WO2020151756 is used for the treatment provided herein. In one embodiment, Form F of Compound A as described in WO2020151756 is used for the treatment provided herein. In one embodiment, Form F of Compound A as described in Example 7 of WO2020151756 is used for the treatment provided herein. The disclosure of WO2020151756 is incorporated herein by reference in its entirety.

As used herein, the term “neoplasm” refers to an abnormal growth of cells or tissue and is understood to include benign, i.e., non-cancerous growths, and malignant, i.e., cancerous growths. The term “neoplastic” means of or related to a neoplasm.

As used herein, the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

By the term “treating” and derivatives thereof as used herein, is meant therapeutic therapy. In reference to a particular condition, treating means: (1) to ameliorate the condition or one or more of the biological manifestations of the condition; (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof, or (4) to slow the progression of the condition or one or more of the biological manifestations of the condition.

As used herein, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that elicits the biological or medical response of a tissue, system, animal, or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compound A disclosed herein may contain one or more chiral atoms, or may otherwise be capable of existing as enantiomers. Accordingly, the compounds of this invention include mixtures of enantiomers as well as purified enantiomers or enantiomerically enriched mixtures. Also, it is understood that all tautomers and mixtures of tautomers are included within the scope of Compound A.

As used herein, the term “solvate” refers to a complex of variable stoichiometry formed by a solute (in this invention, compounds of formula (I) or a salt thereof and a solvent). Also, it is understood that Compound A may be presented, separately or both, as solvates. Such solvents for the purpose of the invention may not interfere with the biological activity of the solute. Examples of suitable solvents include, but are not limited to, water, methanol, dimethylsulforide. ethanol and acetic acid. In one embodiment, the solvent used is a pharmaceutically acceptable solvent. Examples of suitable pharmaceutically acceptable solvents include, without limitation, water, ethanol and acetic acid. In another embodiment, the solvent used is water (i.e., a hydrate).

Compound A may have the ability to crystallize in more than one form, a characteristic, which is known polymorphism, and it is understood that such polymorphic forms (“polymorphs”) are within the scope of Compound A. Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percentages of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 2θ (or ±0.2 degrees 2θ) while still describing the particular XRPD peak.

As used herein, the term “pharmaceutically acceptable salt(s)” refers to a salt prepared from a pharmaceutically acceptable non-toxic acid or base, including an inorganic acid and base and an organic acid and base. Suitable pharmaceutically acceptable base addition salts of the compounds provided herein include, but are not limited to those well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center is substantially free of the opposite enantiomer of the compound. A stereomerically pure compound having two chiral centers is substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

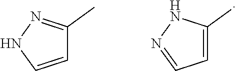

As readily understood by one skilled in the art, a wide variety of functional groups and other structures may exhibit tautomerism and all tautomers of the compounds provided herein are within the scope of the present invention.

It should also be noted the compounds can contain unnatural proportions of atomic isotopes at one or more of the atoms. For example, the compounds may be radiolabeled with radioactive isotopes, such as for example tritium (³H), iodine-125 (¹²³I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

The term “subject” includes an animal, including, but not limited to, an animal such as a primate, a cow, monkey, horse, sheep, pig, chicken, turkey, quail, cat, dog, mouse, rat, rabbit, or guinea pig. In some embodiments, the subject is a mammal, for example, a human.

While it is possible that, for use in therapy, Compound A, may be administered as the raw chemical, it is possible to present the active ingredient as a pharmaceutical composition. Accordingly, the invention further provides pharmaceutical compositions, which include a Compound A, and one or more pharmaceutically acceptable carriers, diluents, or excipients. Compound A is as described above. The carrier(s), diluent(s) or excipient(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical composition including admixing a Compound A with one or more pharmaceutically acceptable carriers, diluents, or excipients. Such elements of the pharmaceutical compositions utilized may be presented in separate pharmaceutical combinations or formulated together in one pharmaceutical composition. Accordingly, the invention further provides a pharmaceutical composition containing Compound A and one or more pharmaceutically acceptable carriers, diluents, or excipients. Compound A described above may be utilized in any of the compositions described above.

Pharmaceutical compositions may be presented in unit dose forms containing a predetermined amount of active ingredient per unit dose. As is known to those skilled in the art, the amount of active ingredient per dose depends on the condition being treated, the route of administration and the age, weight and condition of the patient. Preferred unit dosage compositions are those containing a daily dose or sub-dose, or an appropriate fraction thereof, of an active ingredient. Furthermore, such pharmaceutical compositions may be prepared by any of the methods well known in the pharmacy art.

The combination may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It is appreciated that the preferred route may vary with, for example, the condition of the recipient of the combination and the cancer to be treated. It is also appreciated that each of the agents administered may be administered by the same or different routes and that the combination provided herein may be compounded together in a pharmaceutical composition, or separated in two pharmaceutical compositions.

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Unless otherwise defined, in all dosing protocols described herein, the regimen of compounds administered does not have to commence with the start of treatment and terminate with the end of treatment; it is only required that the number of consecutive days in which both compounds are administered and the optional number of consecutive days in which only one of the component compounds is administered, or the indicated dosing protocol—including the amount of compound administered, occur at some point during the course of treatment.

Compound A may be employed in combination with an anti-EGFR antibody or an antigen binding fragment thereof in accordance with the disclosure by administration simultaneously in a unitary pharmaceutical composition including both compounds.

Furthermore, it does not matter if the compounds are administered in the same dosage form, e.g., Compound A may be administered orally and the anti-EGFR antibody or an antigen binding fragment thereof may be administered intravenously.

By the term “kit” or “kit of parts” as used herein is meant the pharmaceutical composition or combination that are used to administer the combination according to the disclosure. In one embodiment, the kit can contain the combination in a single pharmaceutical composition, such as a tablet, or in separate pharmaceutical compositions. In one aspect, there is provided a kit of parts comprising components: the combination in association with a pharmaceutically acceptable excipients, diluents, or carrier. The kit can also be provided with instruction, such as dosage and administration instructions. Such dosage and administration instructions can be of the kind that are provided to a doctor, for example by a drug product label, or they can be of the kind that are provided by a doctor, such as instructions to a patient.

The term “dose” as used herein is understood to mean a dose that is intended to either slowly raise plasma or blood concentration levels of the compound to a therapeutically effective level, or to maintain such a therapeutically effective level.

In certain embodiments, the treatment of a cancer may be assessed by Response Evaluation Criteria in Solid Tumors (RECIST 1.1) (see Thereasse P., et al. New Guidelines to Evaluate the Response to Treatment in Solid Tumors. J. of the National Cancer Institute; 2000; (92) 205-216 and Eisenhauer, Elizabeth A., et al., European journal of cancer 45.2 (2009): 228-247). New response evaluation criteria in solid tumors: Revised RECIST guideline (version 1.1). European J. Cancer; 2009; (45) 228-247). Overall responses for all possible combinations of tumor responses in target and non-target lesions with or without the appearance of new lesions are as follows:

| Target | | | Overall |
|---|---|---|---|
| lesions | Non-target lesions | New lesions | response |
| CR | CR | No | CR |
| CR | Incomplete response/SD | No | PR |
| PR | Non-PD | No | PR |
| SD | Non-PD | No | SD |
| PD | Any | Yes or no | PD |
| Any | PD | Yes or no | PD |
| Any | Any | Yes | PD |

CR = complete response; PR = partial response; SD = stable disease; and PD = progressive disease.

With respect to the evaluation of target lesions, complete response (CR) is the disappearance of all target lesions, partial response (PR) is at least a 30% decrease in the sum of the longest diameter of target lesions, taking as reference the baseline sum longest diameter, progressive disease (PD) is at least a 20% increase in the sum of the longest diameter of target lesions, taking as reference the smallest sum longest diameter recorded since the treatment started or the appearance of one or more new lesions, and stable disease (SD) is neither sufficient shrinkage to qualify for partial response nor sufficient increase to qualify for progressive disease, taking as reference the smallest sum longest diameter since the treatment started.

With respect to the evaluation of non-target lesions, complete response (CR) is the disappearance of all non-target lesions and normalization of tumor marker level; incomplete response/stable disease (SD) is the persistence of one or more non-target lesion(s) and/or the maintenance of tumor marker level above the normal limits, and progressive disease (PD) is the appearance of one or more new lesions and/or unequivocal progression of existing non-target lesions.

The procedures, conventions, and definitions described below provide guidance for implementing the recommendations from the Response Assessment for Neuro-Oncology (RANO) Working Group regarding response criteria for high-grade gliomas (Wen P., Macdonald, DR., Reardon, D A., et al. Updated response assessment criteria for high-grade gliomas: Response assessment in neuro-oncology working group. J Clin Oncol 2010; 28: 1963-1972). Primary modifications to the RANO criteria for Criteria for Time Point Responses (TPR) can include the addition of operational conventions for defining changes in glucocorticoid dose, and the removal of subjects' clinical deterioration component to focus on objective radiologic assessments. The baseline MRI scan is defined as the assessment performed at the end of the post-surgery rest period, prior to initiating or re-initiating compound treatment. The baseline MRI is used as the reference for assessing complete response (CR) and partial response (PR). Whereas, the smallest SPD (sum of the products of perpendicular diameters) obtained either at baseline or at subsequent assessments are designated the nadir assessment and utilized as the reference for determining progression. For the 5 days preceding any protocol-defined MRI scan, subjects receive either no glucocorticoids or are on a stable dose of glucocorticoids. A stable dose is defined as the same daily dose for the 5 consecutive days preceding the MRI scan. If the prescribed glucocorticoid dose is changed in the 5 days before the baseline scan, a new baseline scan is required with glucocorticoid use meeting the criteria described above. The following definitions are used.

Measurable Lesions: Measurable lesions are contrast-enhancing lesions that can be measured bi-dimensionally. A measurement is made of the maximal enhancing tumor diameter (also known as the longest diameter, LD). The greatest perpendicular diameter is measured on the same image. The cross hairs of bi-dimensional measurements should cross and the product of these diameters are calculated.

Minimal Diameter: T1-weighted image in which the sections are 5 mm with 1 mm skip. The minimal LD of a measurable lesion is set as 5 mm by 5 mm. Larger diameters may be required for inclusion and/or designation as target lesions. After baseline, target lesions that become smaller than the minimum requirement for measurement or become no longer amenable to bi-dimensional measurement are recorded at the default value of 5 mm for each diameter below 5 mm. Lesions that disappear are recorded as 0 mm by 0 mm.

Multicentric Lesions: Lesions that are considered multicentric (as opposed to continuous) are lesions where there is normal intervening brain tissue between the two (or more) lesions. For multicentric lesions that are discrete foci of enhancement, the approach is to separately measure each enhancing lesion that meets the inclusion criteria. If there is no normal brain tissue between two (or more) lesions, they are considered the same lesion.

Nonmeasurable Lesions: All lesions that do not meet the criteria for measurable disease as defined above are considered non-measurable lesions, as well as all non-enhancing and other truly nonmeasurable lesions. Nonmeasurable lesions include foci of enhancement that are less than the specified smallest diameter (i.e., less than 5 mm by 5 mm), non-enhancing lesions (e.g., as seen on T1-weighted post-contrast, T2-weighted, or fluid-attenuated inversion recovery (FLAIR) images), hemorrhagic or predominantly cystic or necrotic lesions, and leptomeningeal tumor. Hemorrhagic lesions often have intrinsic T1-weighted hyperintensity that could be misinterpreted as enhancing tumor, and for this reason, the pre-contrast T1-weighted image may be examined to exclude baseline or interval sub-acute hemorrhage.

At baseline, lesions are classified as follows: Target lesions: Up to 5 measurable lesions can be selected as target lesions with each measuring at least 10 mm by 5 mm, representative of the subject's disease; Non-target lesions: All other lesions, including all nonmeasurable lesions (including mass effects and T2/FLAIR findings) and any measurable lesion not selected as a target lesion. At baseline, target lesions are to be measured as described in the definition for measurable lesions and the SPD of all target lesions is to be determined. The presence of all other lesions is to be documented. At all post-treatment evaluations, the baseline classification of lesions as target and non-target lesions are maintained and lesions are documented and described in a consistent fashion over time (e.g., recorded in the same order on source documents and eCRFs). All measurable and nonmeasurable lesions must be assessed using the same technique as at baseline (e.g., subjects should be imaged on the same MRI scanner or at least with the same magnet strength) for the duration of the study to reduce difficulties in interpreting changes. At each evaluation, target lesions are measured and the SPD calculated. Non-target lesions are assessed qualitatively and new lesions, if any, are documented separately. At each evaluation, a time point response is determined for target lesions, non-target lesions, and new lesion. Tumor progression can be established even if only a subset of lesions is assessed. However, unless progression is observed, objective status (stable disease, PR or CR) can only be determined when all lesions are assessed.

Confirmation assessments for overall time point responses of CR and PR are performed at the next scheduled assessment, but confirmation may not occur if scans have an interval of <28 days. Best response, incorporating confirmation requirements, are derived from the series of time points.

As used herein, “in some embodiments”, “in one embodiment”, and “in certain embodiments” are all used interchangeably. In some embodiments, recitations of Compound A may be replaced with “Compound A or a pharmaceutically acceptable salt or solvate thereof” or “Compound A or a pharmaceutically acceptable salt, tautomer, stereoisomer, enantiomer, isotopologue, solvate, or prodrug” and vice versa.

As used herein, all amounts specified for Compound A or panitumumab are indicated as the amount of free or unsalted compound.

### Combination

Provided here is a combination of Compound A having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or the structure of formula (I):

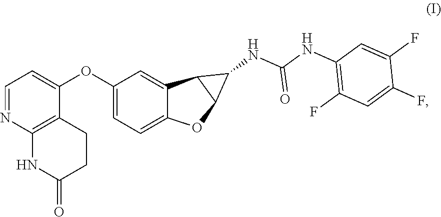 [SMILES: O=C1CCc2c(Oc3ccc4c(c3)[C@H]3[C@H](NC(=O)Nc5cc(F)c(F)cc5F)[C@H]3O4)ccnc2N1]

or a pharmaceutically acceptable salt, tautomer, stereoisomer, enantiomer, isotopologue, solvate, or prodrug thereof, and an anti-EGFR antibody or an antigen-binding fragment thereof. The combination is useful in the treatment of cancer. In some embodiments, the anti-EGFR antibody is panitumumab, cetuximab, zalutumumab, nimotuzumab, or matuzumab or an antigen binding fragment thereof. In one embodiment, the anti-EGFR antibody is panitumumab or an antigen binding fragment thereof. In one embodiment, the combination comprises an anti-EGFR antibody. In one embodiment, the combination comprises panitumumab.

In one embodiment, a solid form of Compound A described in WO2020151756 is used for the treatment provided herein. In one embodiment, Forms A, A*, A**, B, C, D, E, F, G, H, I, J, or K of Compound A as described in WO2020151756 is used for the treatment provided herein. In one embodiment, Form F of Compound A as described in WO2020151756 is used for the treatment provided herein. In one embodiment, Compound A is in Form F.

In one embodiment, a combination kit comprising a combination provided herein together with a pharmaceutically acceptable carrier or carriers. In one embodiment, provided herein is the method of treatment provided herein with a combination kit comprising a combination provided herein. In one embodiment, provided herein is the use provided herein of a combination kit comprising a combination provided herein.

In one embodiment, the anti-EGFR antibody, e.g., panitumumab, is provided in a form suitable for IV administration.

In one embodiment, the anti-EGFR antibody, e.g., panitumumab, is provided in a form suitable for subcutaneous administration.

### Methods of Treatment

Provided herein is a method of treating a cancer in a subject in need thereof, comprising administering to the subject a combination disclosed herein. Provided herein is a combination disclosed herein for use in treating or preventing cancer. Provided herein is use of a combination disclosed herein in the manufacture of medicament for treating or preventing cancer.

Also provided herein is Compound A, or a pharmaceutically acceptable salt, tautomer, stereoisomer, enantiomer, isotopologue, solvate, or prodrug thereof, for use in treating or preventing (e.g., treating) cancer in a subject, wherein Compound A, or a pharmaceutically acceptable salt, tautomer, stereoisomer, enantiomer, isotopologue, solvate, or prodrug thereof, is to be administered in combination with an anti-EGFR antibody. In some embodiments, the anti-EGFR antibody is panitumumab.

Also provided herein is a pharmaceutical composition comprising Compound A, or a pharmaceutically acceptable salt, tautomer, stereoisomer, enantiomer, isotopologue, solvate, or prodrug thereof, for use in treating or preventing (e.g., treating) cancer in a subject, wherein Compound A, or a pharmaceutically acceptable salt, tautomer, stereoisomer, enantiomer, isotopologue, solvate, or prodrug thereof, is to be administered in combination with an anti-EGFR antibody. In some embodiments, the anti-EGFR antibody is panitumumab.

Also provided herein is an anti-EGFR antibody for use in treating or preventing (e.g., treating) cancer, wherein the anti-EGFR antibody is to be administered in combination with Compound A, or a pharmaceutically acceptable salt, tautomer, stereoisomer, enantiomer, isotopologue, solvate, or prodrug thereof. In some embodiments, the anti-EGFR antibody is panitumumab.

Also provided herein is a pharmaceutical composition comprising an anti-EGFR antibody for use in treating or preventing (e.g., treating) cancer, wherein the anti-EGFR antibody is to be administered in combination with Compound A, or a pharmaceutically acceptable salt, tautomer, stereoisomer, enantiomer, isotopologue, solvate, or prodrug thereof. In some embodiments, the anti-EGFR antibody is panitumumab.

Also provided herein is the use of Compound A, or a pharmaceutically acceptable salt, tautomer, stereoisomer, enantiomer, isotopologue, solvate, or prodrug thereof, for the manufacture of a medicament for treating or preventing (e.g., treating) cancer, wherein the medicament is to be adminstered with an anti-EGFR antibody. In some embodiments, the anti-EGFR antibody is panitumumab.

Also provided herein is the use of an anti-EGFR antibody for the manufacture of a medicament for treating or preventing (e.g., treating) cancer, wherein the medicament is to be adminstered with Compound A, or a pharmaceutically acceptable salt, tautomer, stereoisomer, enantiomer, isotopologue, solvate, or prodrug thereof. In some embodiments, the anti-EGFR antibody is panitumumab.

Further provided herein is a method of treating cancer in a human in need thereof comprising the administration of a therapeutically effective amount of Compound A; and an anti-EGFR antibody. In some embodiments, the anti-EGFR antibody is panitumumab, cetuximab, zalutumumab, nimotuzumab, or matuzumab or an antigen binding fragment thereof. In one embodiment, the anti-EGFR antibody is panitumumab or an antigen binding fragment thereof. In one embodiment, the administered anti-EGFR antibody is panitumumab.

In some embodiments, Compound A or a pharmaceutically acceptable salt, tautomer, stereoisomer, enantiomer, isotopologue, solvate, or prodrug thereof is administered at about 5 mg, about 10 mg, about 15 mg, about 20 mg, about 25 mg, about 30 mg, about 35 mg, about 40 mg, about 45 mg, about 50 mg, about 55 mg, or about 60 mg per day. In some embodiments, Compound A or a pharmaceutically acceptable salt, tautomer, stereoisomer, enantiomer, isotopologue, solvate, or prodrug thereof is administered orally at about 5 mg, about 10 mg, about 15 mg, about 20 mg, about 25 mg, about 30 mg, about 35 mg, about 40 mg, about 45 mg, about 50 mg, about 55 mg, or about 60 mg per day. In some embodiments, Compound A or a pharmaceutically acceptable salt, tautomer, stereoisomer, enantiomer, isotopologue, solvate, or prodrug thereof is administered at about 10±5 mg, about 15±5 mg, about 20±5 mg, about 25±5 mg, about 30±5 mg, about 35±5 mg, about 40±5 mg, about 45±5 mg, about 50±5 mg, about 55±5 mg, or about 60±5 mg per day. In some embodiments, Compound A or a pharmaceutically acceptable salt, tautomer, stereoisomer, enantiomer, isotopologue, solvate, or prodrug thereof is administered at about 5±3 mg, about 10±3 mg, about 15±3 mg, about 20±3 mg, about 25±3 mg, about 30±3 mg, about 35±3 mg, about 40±3 mg, about 45±3 mg, about 50±3 mg, about 55±3 mg, or about 60±3 mg per day. In some embodiments, Compound A or a pharmaceutically acceptable salt, tautomer, stereoisomer, enantiomer, isotopologue, solvate, or prodrug thereof is administered at about 5±1 mg, about 10±1 mg, about 15±1 mg, about 20±1 mg, about 25±1 mg, about 30±1 mg, about 35±1 mg, about 40±1 mg, about 45±1 mg, about 50±1 mg, about 55±1 mg, or about 60±1 mg per day. In some embodiments, Compound A or a pharmaceutically acceptable salt, tautomer, stereoisomer, enantiomer, isotopologue, solvate, or prodrug thereof is administered at a dose between about 5 mg and about 60 mg per day. In some embodiments, Compound A or a pharmaceutically acceptable salt, tautomer, stereoisomer, enantiomer, isotopologue, solvate, or prodrug thereof is administered at a dose between about 5 mg and about 40 mg per day. In some embodiments, Compound A or a pharmaceutically acceptable salt, tautomer, stereoisomer, enantiomer, isotopologue, solvate, or prodrug thereof is administered at about 5 mg per day. In some embodiments, Compound A or a pharmaceutically acceptable salt, tautomer, stereoisomer, enantiomer, isotopologue, solvate, or prodrug thereof is administered at about 10 mg per day. In some embodiments, Compound A or a pharmaceutically acceptable salt, tautomer, stereoisomer, enantiomer, isotopologue, solvate, or prodrug thereof is administered at about 15 mg per day. In some embodiments, Compound A or a pharmaceutically acceptable salt, tautomer, stereoisomer, enantiomer, isotopologue, solvate, or prodrug thereof is administered at about 20 mg per day. In some embodiments, Compound A or a pharmaceutically acceptable salt, tautomer, stereoisomer, enantiomer, isotopologue, solvate, or prodrug thereof is administered at about 25 mg per day. In some embodiments, Compound A or a pharmaceutically acceptable salt, tautomer, stereoisomer, enantiomer, isotopologue, solvate, or prodrug thereof is administered at about 30 mg per day. In some embodiments, Compound A or a pharmaceutically acceptable salt, tautomer, stereoisomer, enantiomer, isotopologue, solvate, or prodrug thereof is administered at about 35 mg per day. In some embodiments, Compound A or a pharmaceutically acceptable salt, tautomer, stereoisomer, enantiomer, isotopologue, solvate, or prodrug thereof is administered at about 40 mg per day. In some embodiments, the administration is oral administration.

In some embodiments, Compound A or a pharmaceutically acceptable salt, tautomer, stereoisomer, enantiomer, isotopologue, solvate, or prodrug thereof is administered once a day. In some embodiments, Compound A or a pharmaceutically acceptable salt, tautomer, stereoisomer, enantiomer, isotopologue, solvate, or prodrug thereof is administered twice a day. In some embodiments, Compound A or a pharmaceutically acceptable salt, tautomer, stereoisomer, enantiomer, isotopologue, solvate, or prodrug thereof is administered three time a day.

In some embodiments, panitumumab is administered in an amount of about 6 mg/kg. In some embodiments, panitumumab is administered intravenously in an amount of about 6 mg/kg. In one embodiment, panitumumab is administered in an amount of about 6 mg/kg administered as an intravenous infusion over about 60 minutes. In one embodiment, panitumumab is administered in an amount of about 6 mg/kg administered as an intravenous infusion over about 60 minutes approximately every two weeks.

In one embodiment, panitumumab is administered in an amount of about 1 mg/kg, about 2 mg/kg, about 3 mg/kg, about 4 mg/kg, about 5 mg/kg, about 6 mg/kg, about 7 mg/kg, or about 8 mg/kg approximately every two weeks. In one embodiment, panitumumab is administered in an amount of about 1 mg/kg±0.5 mg/kg, about 2 mg/kg±0.5 mg/kg, about 3 mg/kg±0.5 mg/kg, about 4 mg/kg±0.5 mg/kg, about 5 mg/kg±0.5 mg/kg, about 6 mg/kg±0.5 mg/kg, about 7 mg/kg±0.5 mg/kg, or about 8 mg/kg±0.5 mg/kg approximately every two weeks. In one embodiment, panitumumab is administered in an amount of about 1 mg/kg±0.3 mg/kg, about 2 mg/kg±0.3 mg/kg, about 3 mg/kg±0.3 mg/kg, about 4 mg/kg±0.3 mg/kg, about 5 mg/kg±0.3 mg/kg, about 6 mg/kg±0.3 mg/kg, about 7 mg/kg±0.3 mg/kg, or about 8 mg/kg±0.3 mg/kg approximately every two weeks. In one embodiment, panitumumab is administered in an amount of about 1 mg/kg±0.1 mg/kg, about 2 mg/kg±0.1 mg/kg, about 3 mg/kg±0.1 mg/kg, about 4 mg/kg±0.1 mg/kg, about 5 mg/kg±0.1 mg/kg, about 6 mg/kg±0.1 mg/kg, about 7 mg/kg±0.1 mg/kg, or about 8 mg/kg±0.1 mg/kg approximately every two weeks.

In one embodiment, panitumumab is administered in an amount of about 1 mg/kg, about 2 mg/kg, about 3 mg/kg, about 4 mg/kg, about 5 mg/kg, about 6 mg/kg, about 7 mg/kg, or about 8 mg/kg administered as an intravenous infusion over about 60 minutes approximately every two weeks. In one embodiment, panitumumab is administered in an amount of about 1 mg/kg±0.5 mg/kg, about 2 mg/kg±0.5 mg/kg, about 3 mg/kg±0.5 mg/kg, about 4 mg/kg±0.5 mg/kg, about 5 mg/kg±0.5 mg/kg, about 6 mg/kg±0.5 mg/kg, about 7 mg/kg±0.5 mg/kg, or about 8 mg/kg±0.5 mg/kg administered as an intravenous infusion over about 60 minutes approximately every two weeks. In one embodiment, panitumumab is administered in an amount of about 1 mg/kg±0.3 mg/kg, about 2 mg/kg±0.3 mg/kg, about 3 mg/kg±0.3 mg/kg, about 4 mg/kg±0.3 mg/kg, about 5 mg/kg±0.3 mg/kg, about 6 mg/kg±0.3 mg/kg, about 7 mg/kg±0.3 mg/kg, or about 8 mg/kg±0.3 mg/kg administered as an intravenous infusion over about 60 minutes approximately every two weeks. In one embodiment, panitumumab is administered in an amount of about 1 mg/kg±0.1 mg/kg, about 2 mg/kg±0.1 mg/kg, about 3 mg/kg±0.1 mg/kg, about 4 mg/kg±0.1 mg/kg, about 5 mg/kg±0.1 mg/kg, about 6 mg/kg±0.1 mg/kg, about 7 mg/kg±0.1 mg/kg, or about 8 mg/kg±0.1 mg/kg administered as an intravenous infusion over about 60 minutes approximately every two weeks.

In some embodiments, panitumumab is administered in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg. In some embodiments, panitumumab is administered in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg.

In one embodiment, panitumumab is coadministered with Compound A or a pharmaceutically acceptable salt or solvate thereof. In one embodiment, panitumumab and Compound A or a pharmaceutically acceptable salt or solvate thereof are administered simultaneously, sequentially or separately. In some embodiments, panitumumab and Compound A or a pharmaceutically acceptable salt or solvate thereof are administered within about 30 minutes of each other. In some embodiments, panitumumab is administered intravenously in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg; and Compound A is administered orally once per day at about 5 mg, about 10 mg, about 15 mg, about 20 mg, about 25 mg, about 30 mg, about 35 mg, or about 40 mg. In some embodiments, panitumumab is administered intravenously in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg; and Compound A is administered orally once per day at about 5 mg. In some embodiments, panitumumab is administered intravenously in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg; and Compound A is administered orally once per day at about 10 mg. In some embodiments, panitumumab is administered intravenously in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg; and Compound A is administered orally once per day at about 15 mg. In some embodiments, panitumumab is administered intravenously in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg; and Compound A is administered orally once per day at about 20 mg. In some embodiments, panitumumab is administered intravenously in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg; and Compound A is administered orally once per day at about 25 mg. In some embodiments, panitumumab is administered intravenously in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg; and Compound A is administered orally once per day at about 30 mg. In some embodiments, panitumumab is administered intravenously in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg; and Compound A is administered orally once per day at about 35 mg. In some embodiments, panitumumab is administered intravenously in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg; and Compound A is administered orally once per day at about 40 mg.

In some embodiments, the cancer is colorectal cancer, pancreatic cancer, or non-small cell lung cancer. In one embodiment, the cancer is colorectal cancer. In one embodiment, the cancer is metastatic colorectal cancer. In one embodiment, the cancer is BRAF mutant metastatic colorectal cancer. In one embodiment, the cancer is BRAF V600E mutant metastatic colorectal cancer. In one embodiment, the cancer is KRAS mutant colorectal cancer. In one embodiment, the cancer is KRAS G12C mutant colorectal cancer. In one embodiment, the cancer is KRAS G12D mutant colorectal cancer. In one embodiment, the cancer is KRAS G12V mutant colorectal cancer. In one embodiment, the cancer is Trp53 mutant colorectal cancer. In one embodiment, the cancer is NRAS mutant colorectal cancer.

In some embodiments, provided herein is a method of treating a colorectal cancer in a subject by administering to the subject an anti-EGFR antibody and Compound A. In some embodiments, the colorectal cancer has an oncogenic K-RAS, N-RAS or B-RAF mutation. In some embodiments, the colorectal cancer is a metastatic or unresectable colorectal cancer. In some embodiments, prior to the administering, the subject had been treated with another therapy and experienced progression of cancer after said another therapy.

In one embodiment, the cancer is pancreatic cancer. In one embodiment, the cancer is pancreatic ductal adenocarcinoma (PDAC). In one embodiment, the cancer is BRAF mutant pancreatic cancer. In one embodiment, the cancer is BRAF V600E mutant pancreatic cancer. In one embodiment, the cancer is KRAS mutant pancreatic cancer. In one embodiment, the cancer is KRAS G12C mutant pancreatic cancer. In one embodiment, the cancer is KRAS G12D mutant pancreatic cancer. In one embodiment, the cancer is KRAS G12V mutant pancreatic cancer. In one embodiment, the cancer is Trp53 mutant pancreatic cancer. In one embodiment, the cancer is NRAS mutant pancreatic cancer.

In one embodiment, the cancer is non-small cell lung cancer. In one embodiment, the cancer is BRAF mutant non-small cell lung cancer. In one embodiment, the cancer is BRAF V600E mutant non-small cell lung cancer. In one embodiment, the cancer is KRAS mutant non-small cell lung cancer. In one embodiment, the cancer is KRAS G12C mutant non-small cell lung cancer. In one embodiment, the cancer is KRAS G12D mutant non-small cell lung cancer. In one embodiment, the cancer is KRAS G12V mutant non-small cell lung cancer. In one embodiment, the cancer is Trp53 mutant non-small cell lung cancer. In one embodiment, the cancer is NRAS mutant non-small cell lung cancer.

In some embodiments, the cancer is colorectal cancer, pancreatic cancer, non-small cell lung cancer, melanoma, brain cancer, lung cancer, kidney cancer, bone cancer, liver cancer, bladder cancer, breast, head and neck cancer, ovarian cancer, skin cancer, adrenal cancer, cervical cancer, lymphoma, or thyroid cancer. In some embodiments, the patient has progressed on one or more prior therapies.

In some embodiments, the cancer is characterized by a mutation in a gene selected from RAS, NRAS, KRAS, RAF, BRAF, CRAF, ARAF, and combination of any thereof; preferably RAS, NRAS, KRAS, RAF, BRAF, and combination of any thereof; more preferably NRAS, KRAS, BRAF, and combination of any thereof. In some embodiments, the cancer is characterized by a mutation in a gene selected from RAS, NRAS, KRAS, RAF, BRAF, CRAF, ARAF, and combination of any thereof, preferably RAS, NRAS, KRAS, RAF, BRAF, and combination of any thereof, more preferably NRAS, KRAS, BRAF, and combination of any thereof, wherein the patient has progressed on one or more prior therapies.

In some embodiments, the cancer is characterized by a mutation selected from NRAS Q61R, NRAS Q61K, NRAS Q61L, NRAS G12S, NRAS G13R, KRAS G12A, KRAS G12C, KRAS G12D, KRAS G12V, BRAF V600E, BRAF fusion, and combination of any thereof; preferably NRAS Q61R, NRAS Q61K, NRAS Q61L, KRAS G12D, KRAS G12V, BRAF V600E, BRAF fusion, and combination of any thereof; more preferably NRAS Q61R, NRAS Q61K, NRAS Q61L, KRAS G12D, KRAS G12V, and combination of any thereof.

In one embodiment, the cancer is characterized by other MAPK pathway genomic aberration. In one embodiment, the other MAPK pathway genomic aberration is RAS A1 splice isoform.

In some embodiments, the cancer is characterized by a mutation in a gene selected from ARAF, BRAF, RAF1, KRAS, HRAS, NF1, MAP2K1, MAP2K2, MAPK1, and combination of any thereof.

In some embodiments, the cancer is characterized by a mutation selected from BRAF N20T, BRAF A33T, BRAF S36A, BRAF V47_G393del, BRAF V47_G327del, BRAF V47_D380del, BRAF V47_M438del, BRAF N49I, BRAF M53I, BRAF L64I, BRAF G69S, BRAF A81_D380del, BRAF A81_M438del, BRAF G104E, BRAF T119S, BRAF P141L, BRAF S151A, BRAF P162S, BRAF V169_G327del, BRAF V169_D380del, BRAF R188T, BRAF Q201H, BRAF G203_G393del, BRAF K205Q, BRAF V226L, BRAF E228V, BRAF R239Q, BRAF T241P, BRAF T241M, BRAF L245F, BRAF A246P, BRAF F247L, BRAF Q257R, BRAF Q257H, BRAF G258V, BRAF F259L, BRAF Q262R, BRAF H269Y, BRAF R271H, BRAF E275K, BRAF D287H, BRAF F294L, BRAF T310I, BRAF A320T, BRAF I326V, BRAF P341S, BRAF R347*, BRAF P348T, BRAF S363F, BRAF S364L, BRAF P367S, BRAF P367R, BRAF P367L, BRAF D380H, BRAF R389C, BRAF T401I, BRAF A404Cfs*9, BRAF P407L, BRAF S419Y, BRAF G421V, BRAF R444W, BRAF D448Y, BRAF D449Y, BRAF W450*, BRAF W450L, BRAF E451K, BRAF E451Q, BRAF P453T, BRAF V459L, BRAF R462E, BRAF R462K, BRAF R462L, BRAF I463T, BRAF I463S, BRAF G464L, BRAF G464R, BRAF G464E, BRAF G464A, BRAF G464V, BRAF S465D, BRAF S465E, BRAF S465A, BRAF G466R, BRAF G466E, BRAF G466A, BRAF G466V, BRAF S467A, BRAF S467L, BRAF F468C, BRAF G469L, BRAF G469del, BRAF G469S, BRAF G469R, BRAF G469E, BRAF G469A, BRAF G469V, BRAF T470K, BRAF V471I, BRAF V471F, BRAF Y472dup, BRAF Y472S, BRAF Y472C, BRAF G478C, BRAF K483E, BRAF K483M, BRAF L485_P490del, BRAF L485Y, BRAF L485_P490delinsY, BRAF L485S, BRAF L485W, BRAF L485F, BRAF L485_P490delinsF, BRAF N486_Q494del, BRAF N486del, BRAF N486_T488del, BRAF N486_T491del, BRAF N486_L495del, BRAF N486D, BRAF N486_V487del, BRAF N486_P490del, BRAF N486_A489delinsK, BRAF N486_T491delinsK, BRAF V487_P490del, BRAF V487_P492delinsA, BRAF T488_P492del, BRAF T488_Q493delinsK, BRAF A489_P490del, BRAF P490del, BRAF P490_Q494del, BRAF K499E, BRAF K499N, BRAF E501K, BRAF E501G, BRAF V504_R506dup, BRAF V504L, BRAF L505F, BRAF L505H, BRAF R509G, BRAF R509H, BRAF L514V, BRAF M517L, BRAF Q524L, BRAF L525R, BRAF T529M, BRAF T529N, BRAF T529I, BRAF W531C, BRAF G534D, BRAF Y538H, BRAF R558Q, BRAF G563D, BRAF H568D, BRAF H574N, BRAF H574Y, BRAF H574Q, BRAF N581D, BRAF N581Y, BRAF N581T, BRAF N581S, BRAF N581I, BRAF N581K, BRAF I582M, BRAF F583C, BRAF L584F, BRAF H585Y, BRAF E586K, BRAF D587A, BRAF D587G, BRAF D587E, BRAF V590L, BRAF V590G, BRAF I592V, BRAF I592M, BRAF G593D, BRAF D594N, BRAF D594H, BRAF D594Y, BRAF D594_T599dup, BRAF D594A, BRAF D594G, BRAF D594V, BRAF D594E, BRAF F595L, BRAF F595S, BRAF G596S, BRAF G596R, BRAF G596C, BRAF G596D, BRAF G596V, BRAF L597S, BRAF L597V, BRAF L597Q, BRAF L597P, BRAF L597R, BRAF A598T, BRAF A598S, BRAF A598V, BRAF A598_T599insARC, BRAF A598_T599insV, BRAF T599dup, BRAF T599A, BRAF T599K, BRAF T599R, BRAF T599L, BRAF T599_V600insTT, BRAF T599_V600insS, BRAF T599_V600insETT, BRAF T599_V600insEAT, BRAF V600_K601delinsEN, BRAF V600_S605delinsEISRWR, BRAF V600K, BRAF V600R, BRAF V600Q, BRAF V600dup, BRAF V600delinsYM, BRAF V600M, BRAF V600L, BRAF V600D, BRAF V600_K601delinsE, BRAF V600E, BRAF V600A, BRAF V600G, BRAF K601del, BRAF K601Q, BRAF K601E, BRAF K601_W604del, BRAF K601T, BRAF K601L, BRAF K601_S602delinsNT, BRAF K601N, BRAF S602T, BRAF S602Y, BRAF S602F, BRAF R603*, BRAF W604del, BRAF W604R, BRAF W604G, BRAF S605A, BRAF S605F, BRAF S605E, BRAF S605G, BRAF S605N, BRAF S605L, BRAF G606W, BRAF G606E, BRAF G606A, BRAF G606V, BRAF S607P, BRAF S607F, BRAF H608R, BRAF Q609E, BRAF Q609L, BRAF Q609H, BRAF E611D, BRAF L613F, BRAF G615R, BRAF L618F, BRAF W619R, BRAF S637*, BRAF V639L, BRAF E648Q, BRAF Y656D, BRAF R671Q, BRAF P676S, BRAF L678I, BRAF V681I, BRAF E695K, BRAF K698R, BRAF L711F, BRAF A712T, BRAF R719S, BRAF H725Y, BRAF A728V, BRAF P731T, BRAF P731S, BRAF P731L, BRAF A762E, BRAF A762V, and combination of any thereof.

In some embodiments, the cancer is characterized by a mutation selected from KIAA1549-BRAF fusion, BCAS1-BRAF fusion, CCDC6-BRAF fusion, CDC42BPB-BRAF fusion, FAM131B-BRAF fusion, FXR1-BRAF fusion, GIT2-BRAF fusion, KLHL7-BRAF fusion, RNF130-BRAF fusion, TMEM106B-BRAF fusion, MKRN1-BRAF fusion, AGAP3-BRAF fusion, AGK-BRAF fusion, AKAP9-BRAF fusion, ARMC10-BRAF fusion, CUL1-BRAF fusion, GTF2I-BRAF fusion, PAPSS1-BRAF fusion, PCBP2-BRAF fusion, PPFIBP2-BRAF fusion, SND1-BRAF fusion, TRIM24-BRAF fusion, ZKSCAN1-BRAF fusion, SEPT3-BRAF fusion, and combination of any thereof.

In some embodiments, the cancer is characterized by a mutation selected from NRAS G12A, NRAS G12C, NRAS G12D, NRAS G12N, NRAS G12P, NRAS G12R, NRAS G12S, NRAS G12V, NRAS G12Y, NRAS G13A, NRAS G13C, NRAS G13D, NRAS G13E, NRAS G13N, NRAS G13R, NRAS G13S, NRAS G13V, NRAS A18T, NRAS I24N, NRAS P34L, NRAS Y40*, NRAS Q43*, NRAS T50I, NRAS T58L, NRAS A59G, NRAS A59D, NRAS A59T, NRAS G60E, NRAS G60R, NRAS Q61E, NRAS Q61H, NRAS Q61H, NRAS Q61K, NRAS Q61L, NRAS Q61L, NRAS Q61P, NRAS Q61R, NRAS Q61R, NRAS Q61R, NRAS Q61*, NRAS E63K, NRAS Y64D, NRAS S65C, NRAS R68S, NRAS S89A, NRAS G115Efs*46, NRAS E132K, NRAS K135N, NRAS A146P, NRAS A146T, NRAS A146V, NRAS E162*, and combination of any thereof.

In some embodiments, the cancer harbors one or more of the mutations as described herein. In some embodiments, the subject with the cancer harbors one or more of the mutations as described herein.

In some embodiments, Compound A is administered one to three times a day. In one embodiment, Compound A is administered three times a day. In one embodiment, Compound A is administered twice a day. In one embodiment, Compound A is administered once a day.

In some embodiments, the method as described herein provides a plasma Compound A AUC₈ₕ between about 2,000 ng*h/ml and about 3,200 ng*h/ml in the subject. In one embodiment, the method as described herein provides a plasma Compound A AUC₈ₕ between about 2,128 ng*h/ml and about 3,192 ng*h/ml in the subject. In one embodiment, the method as described herein provides a plasma Compound A AUC₈ₕ between about 2,400 ng*h/ml and about 2,900 ng*h/ml in the subject.

In some embodiments, the method as described herein provides a plasma Compound A AUC₈ₕ between about 4,600 ng*h/ml and about 6,900 ng*h/ml in the subject. In one embodiment, the method as described herein provides a plasma Compound A AUC₈ₕ between about 4,576 ng*h/ml and about 6,864 ng*h/ml in the subject. In one embodiment, the method as described herein provides a plasma Compound A AUC₈ₕ between about 5,100 ng*h/ml and about 6,300 ng*h/ml in the subject.

In some embodiments, the method as described herein provides a plasma Compound A AUC₈ₕ between about 8,000 ng*h/ml and about 12,000 ng*h/ml in the subject. In one embodiment, the method as described herein provides a plasma Compound A AUC₈ₕ between about 7,944 ng*h/ml and about 11,916 ng*h/ml in the subject. In one embodiment, the method as described herein provides a plasma Compound A AUC₈ₕ between about 8,900 ng*h/ml and about 10,900 ng*h/ml in the subject.

In some embodiments, the method as described herein provides a plasma Compound A AUC₈ₕ between about 10,000 ng*h/ml and about 14,800 ng*h/ml in the subject. In one embodiment, the method as described herein provides a plasma Compound A AUC₈ₕ between about 9,840 ng*h/ml and about 14,760 ng*h/ml in the subject. In one embodiment, the method as described herein provides a plasma Compound A AUC₈ₕ between about 11,100 ng*h/ml and about 13,500 ng*h/ml in the subject.

In some embodiments, the method as described herein provides a plasma Compound A AUC₈ₕ between about 12,700 ng*h/ml and about 19,000 ng*h/ml in the subject. In one embodiment, the method as described herein provides a plasma Compound A AUC₈ₕ between about 12,640 ng*h/ml and about 18,960 ng*h/ml in the subject. In one embodiment, the method as described herein provides a plasma Compound A AUC₈ₕ between about 14,200 ng*h/ml and about 17,400 ng*h/ml in the subject.

In some embodiments, the method as described herein provides a plasma Compound A AUC₈ₕ between about 30,000 ng*h/ml and about 45,000 ng*h/ml in the subject. In one embodiment, the method as described herein provides a plasma Compound A AUC₈ₕ between about 33,800 ng*h/ml and about 41,300 ng*h/ml in the subject.

In some embodiments, the method as described herein provides a plasma Compound A AUC₈ₕ between about 2,000 ng*h/ml and about 3,200 ng*h/ml in the subject receiving Compound A treatment at about 5 mg/day. In one embodiment, the method as described herein provides a plasma Compound A AUC₈ₕ between about 2,128 ng*h/ml and about 3,192 ng*h/ml in the subject receiving Compound A treatment at about 5 mg/day. In one embodiment, the method as described herein provides a plasma Compound A AUC₈ₕ between about 2,400 ng*h/ml and about 2,900 ng*h/ml in the subject receiving Compound A treatment at about 5 mg/day.

In some embodiments, the method as described herein provides a plasma Compound A AUC₈ₕ between about 4,600 ng*h/ml and about 6,900 ng*h/ml in the subject receiving Compound A treatment at about 10 mg/day. In one embodiment, the method as described herein provides a plasma Compound A AUC₈ₕ between about 4,576 ng*h/ml and about 6,864 ng*h/ml in the subject receiving Compound A treatment at about 10 mg/day. In one embodiment, the method as described herein provides a plasma Compound A AUC₈ₕ between about 5,100 ng*h/ml and about 6,300 ng*h/ml in the subject receiving Compound A treatment at about 10 mg/day.

In some embodiments, the method as described herein provides a plasma Compound A AUC₈ₕ between about 8,000 ng*h/ml and about 12,000 ng*h/ml in the subject receiving Compound A treatment at about 15 mg/day. In one embodiment, the method as described herein provides a plasma Compound A AUC₈ₕ between about 7,944 ng*h/ml and about 11,916 ng*h/ml in the subject receiving Compound A treatment at about 15 mg/day. In one embodiment, the method as described herein provides a plasma Compound A AUC₈ₕ between about 8,900 ng*h/ml and about 10,900 ng*h/ml in the subject receiving Compound A treatment at about 15 mg/day.

In some embodiments, the method as described herein provides a plasma Compound A AUC₈ₕ between about 10,000 ng*h/ml and about 14,800 ng*h/ml in the subject receiving Compound A treatment at about 25 mg/day. In one embodiment, the method as described herein provides a plasma Compound A AUC₈ₕ between about 9,840 ng*h/ml and about 14,760 ng*h/ml in the subject receiving Compound A treatment at about 25 mg/day. In one embodiment, the method as described herein provides a plasma Compound A AUC₈ₕ between about 11,100 ng*h/ml and about 13,500 ng*h/ml in the subject receiving Compound A treatment at about 25 mg/day.

In some embodiments, the method as described herein provides a plasma Compound A AUC₈ₕ between about 12,700 ng*h/ml and about 19,000 ng*h/ml in the subject receiving Compound A treatment at about 40 mg/day. In one embodiment, the method as described herein provides a plasma Compound A AUC₈ₕ between about 12,640 ng*h/ml and about 18,960 ng*h/ml in the subject receiving Compound A treatment at about 40 mg/day. In one embodiment, the method as described herein provides a plasma Compound A AUC₈ₕ between about 14,200 ng*h/ml and about 17,400 ng*h/ml in the subject receiving Compound A treatment at about 40 mg/day.

In some embodiments, the method as described herein provides a plasma Compound A AUC₈ₕ between about 30,000 ng*h/ml and about 45,000 ng*h/ml in the subject receiving Compound A treatment at about 60 mg/day. In one embodiment, the method as described herein provides a plasma Compound A AUC₈ₕ between about 33,800 ng*h/ml and about 41,300 ng*h/ml in the subject receiving Compound A treatment at about 60 mg/day.

In some embodiments, the AUC₈ₕ is measured in the subject's plasma. In some embodiments, the AUC₈ₕ is measured in the subject's blood. In some embodiments, the AUC₈ₕ is measured in the subject's plasma or blood on Cycle 2 Day 1. In some embodiments, the AUC₈ₕ is measured in the subject's plasma or blood on about Day 29 of treatment with Compound A.

In one embodiment, the subject receives the treatment provided herein for 1 to 12 cycles, wherein every cycle consists of about 28 days. In one embodiment, the subject receives the treatment provided herein for 1 to 12 cycles, wherein every cycle consists of about 21 days. In one embodiment, the subject receives the treatment provided herein for 1 to 12 cycles, wherein every cycle consists of about 14 days. In one embodiment, the subject receives the treatment provided herein for 1 to 12 cycles, wherein every cycle consists of about 7 days.

In one embodiment, the subject receives the treatment provided herein for 1 to 12 cycles, wherein every cycle consists of about 28 days. In one embodiment, the subject receives the treatment provided herein for 1 to 10 cycles, wherein every cycle consists of about 28 days. In one embodiment, the subject receives the treatment provided herein for 1 to 8 cycles, wherein every cycle consists of about 28 days. In one embodiment, the subject receives the treatment provided herein for 1 to 6 cycles, wherein every cycle consists of about 28 days. In one embodiment, the subject receives the treatment provided herein for 1 to 4 cycles, wherein every cycle consists of about 28 days. In one embodiment, the subject receives the treatment provided herein for 4 cycles, wherein every cycle consists of about 28 days. In one embodiment, the subject receives the treatment provided herein for 3 cycles, wherein every cycle consists of about 28 days. In one embodiment, the subject receives the treatment provided herein for 2 cycles, wherein every cycle consists of about 28 days. In one embodiment, the subject receives the treatment provided herein for 1 cycle, wherein every cycle consists of about 28 days.

In one embodiment, the subject achieves a stable disease, a partial response, or a complete response. In one embodiment, the subject achieves a partial response or a complete response. In one embodiment, the subject achieves a complete response. In one embodiment, the subject does not experience a progressive disease. In one embodiment, the subject achieves a stable disease. In one embodiment, the subject achieves a partial response. In one embodiment, the subject achieves a stable disease, a partial response, or a complete response for 1 week, 2 weeks, 3 weeks, or 4 weeks. In one embodiment, the subject achieves a stable disease, a partial response, or a complete response for 1 month, 2 months, 3 months, 4 months, 5 months, 6 months, 7 months, 8 months, 9 months, 10 months, 11 months, 12 months. In one embodiment, the subject achieves a stable disease, a partial response, or a complete response for 1 year, 2 years, 3 years, or 4 years.

Provided herein is a method of treating a cancer in a subject in need thereof, comprising administering to said subject a combination provided herein, wherein the cancer is characterized by a mutation selected from BRAF, RAS, NRAS, KRAS, and their combination thereof, preferably NRAS, and KRAS, and their combination thereof, more preferably KRAS. Provided herein is a method of treating a cancer in a subject in need thereof, comprising administering to said subject an inhibitor of BRAF, wherein the cancer is characterized by a KRAS mutation. In one embodiment, the cancer is colorectal cancer (CRC). In one embodiment, the cancer is PDAC.

In some embodiments, the cancer is colorectal cancer, pancreatic cancer, melanoma, non-small cell lung cancer, brain cancer, lung cancer, kidney cancer, bone cancer, liver cancer, bladder cancer, breast, head and neck cancer, ovarian cancer, skin cancer, adrenal cancer, cervical cancer, lymphoma, or thyroid cancer.

In some embodiments, the cancer is characterized by a mutation selected from NRAS Q61R, NRAS Q61K, NRAS Q61L, NRAS G12S, NRAS G13R, KRAS G12A, KRAS G12C, KRAS G12D, KRAS G12V, BRAF V600E, BRAF fusion, and combination of any thereof; preferably NRAS Q61R, NRAS Q61K, NRAS Q61L, KRAS G12D, KRAS G12V, BRAF V600E, BRAF fusion, and combination of any thereof, more preferably NRAS Q61R, NRAS Q61K, NRAS Q61L, KRAS G12D, KRAS G12V, and combination of any thereof. In one embodiment, the cancer is characterized by a mutation provided herein.

In certain embodiments, the patient has colorectal cancer, pancreatic cancer, non-small cell lung cancer, melanoma, brain cancer, lung cancer, kidney cancer, bone cancer, liver cancer, bladder cancer, breast, head and neck cancer, ovarian cancer, skin cancer, adrenal cancer, cervical cancer, lymphoma, or thyroid cancer. In particular embodiments, the patient response measured is inhibition of disease progression, inhibition of tumor growth, reduction of primary and/or secondary tumor(s), relief of tumor-related symptoms, improvement in quality of life, delayed appearance of primary and/or secondary tumors, slowed development of primary and/or secondary tumors, decreased occurrence of primary and/or secondary tumors, slowed or decreased severity of secondary effects of disease, arrested tumor growth or regression of tumor.

Provided herein is a method of treating a cancer in a subject in need thereof, comprising administering to said subject a combination provided herein, wherein the cancer is metastatic cancer, resistant cancer, recurrent cancer, and/or unresectable cancer. In some embodiments, the subject is an adult patient, e.g., a patient 18 years and older. In one embodiment, Form F is in a powder form with particle sizes of D90<about 200 μm. In one embodiment, Form F is in a powder form with particle sizes of D90<about 400 μm. In one embodiment, Form F is in a powder form with particle sizes of D90<about 600 μm. In one embodiment, Form F is in a powder form with particle sizes of D90<about 100 μm. In one embodiment, Form F is in a powder form with particle sizes of D90<about 50 μm. In one embodiment, the purity of Form F is more than about 99.0%. In one embodiment, the purity of Form F is more than about 98.0%. In one embodiment, the purity of Form F is more than about 97.0%. In one embodiment, the purity of Form F is more than about 96.0%. In one embodiment, the purity of Form F is more than about 95.0%.

### Kits

Provided herein is a kit comprising Compound A, or a pharmaceutically acceptable salt, tautomer, stereoisomer, enantiomer, isotopologue, solvate, or prodrug thereof, and an anti-EGFR antibody, along with instructions for effective administration. For example, in some embodiments, the kit comprises Compound A and panitumumab, along with instructions for effective administration. In some embodiments, the kit is for use in a method (e.g., in a method of treating cancer) described herein.

Provided herein is a kit comprising a combination provided herein and means for monitoring patient response to administration of said compound provided herein.

In other embodiments, provided herein are kits comprising a combination provided herein and means for measuring the amount of inhibition of B-RAF, KRAS, NRAS, or MEK in a patient. In certain embodiments, the kits comprise means for measuring inhibition of B-RAF or MEK in circulating plasma, blood, or tumor cells and/or skin biopsies or tumor biopsies/aspirates of a patient. In certain embodiments, provided herein are kits comprising a compound provided herein and means for measuring the amount of inhibition of B-RAF, KRAS, NRAS, or MEK before, during and/or after administration of a compound provided herein. In certain embodiments, the patient has colorectal cancer, pancreatic cancer, non-small cell lung cancer, melanoma, or ovarian cancer.

In certain embodiments, the kits provided herein comprise an amount of a combination provided herein effective for treating or preventing colorectal cancer, pancreatic cancer, non-small cell lung cancer, melanoma, brain cancer, lung cancer, kidney cancer, bone cancer, liver cancer, bladder cancer, breast, head and neck cancer, ovarian cancer, skin cancer, adrenal cancer, cervical cancer, lymphoma, and thyroid cancer; preferably melanoma, ovarian cancer, and non-small cell lung cancer. In certain embodiments, the kits provided herein comprise an amount of a compound provided herein effective for treating or preventing colorectal cancer, pancreatic cancer, non-small cell lung cancer, melanoma, or ovarian cancer.

In certain embodiments, the kits provided herein further comprise with a pharmaceutically acceptable carrier or carriers.

In certain embodiments, the kits provided herein further comprise instructions for use, such as for administering a compound provided herein and/or monitoring patient response to administration of the compound.

### EXAMPLES

The examples below were intended to be purely exemplary and should not be considered to be limiting in any way. Unless otherwise specified, the experimental methods in the Examples described below were conventional methods.

TABLE 1
List of Abbreviations and Terms

| Abbreviation | Definition |
|---|---|
| AE | adverse event |
| A-RAF | A-RAF proto-oncogene |
| AUC | area under the plasma concentration time curve |
| AUC_{0-∞} | area under the plasma concentration time curve from zero |
| | to infinity |
| AUCₗₐₛₜ | area under the plasma concentration time curve from zero |
| | to the last measurable concentration |
| BID | twice daily |
| B-RAF | v-RAF murine sarcoma viral oncogene homolog B |
| C-RAF | Raf-1 proto-oncogene serine/threonine kinase |
| CI | confidence interval |
| Cmax | maximum observed plasma concentration |
| CR | complete response |
| CRC | colorectal cancer |
| CT | computed tomography |
| CTCAE | Common Terminology Criteria for Adverse Events |
| CYP | cytochrome P450 |
| DLT | dose-limiting toxicity |
| ECG | electrocardiogram |
| ECHO | echocardiogram |
| ECOG | Eastern Cooperative Oncology Group |
| eCRF | electronic case report form |
| EDC | electronic data capture (system) |
| EGFR | epidermal growth factor receptor |
| ERK | extracellular signal-regulated kinase |
| FDG-PET | fluorodeoxyglucose PET |
| HBsAg | hepatitis B surface antigen |
| HCC | hepatocellular carcinoma |
| IB | Investigator's Brochure |
| IC₅₀ | concentration of an inhibitor where the response (or |
| | binding) was reduced by half |
| ICF | informed consent form |
| ICH | International Council for Harmonisation |
| IEC | Independent Ethics Committee |
| IRB | Institutional Review Board |
| K-RAS | Kirsten rat sarcoma virus oncogene |
| LVEF | left ventricular ejection fraction |
| MAPK | mitogen activated protein kinase |
| MedDRA | Medical Dictionary for Regulatory Activities |
| MEK | MAPK/ERK kinase |
| MRI | magnetic resonance imaging |
| MTD | maximum tolerated dose |
| NF1 | neurofibromin 1 |
| N-RAS | neuroblastoma RAS viral oncogene homolog |
| NSCLC | non-small cell lung carcinoma |
| ORR | objective response rate |
| PD | pharmacodynamics |
| PET | positron emission tomography |
| PFS | progression-free survival |
| PK | pharmacokinetic(s) |
| PR | partial response |
| QD | once a day |
| RAF | rapidly accelerated fibrosarcoma |
| RECIST | Response Evaluation Criteria in Solid Tumors |
| RP2D | recommended phase 2 dose |
| RVO | retinal vein occlusion |
| SAE | serious adverse event |
| SMC | Safety Monitoring Committee |
| SS | steady state |
| T_{1/2} | elimination half-life |
| TEAE | treatment-emergent adverse event |
| Tₘₐₓ | time to maximum observed plasma concentration |
| ULN | upper limit of normal |

### Example 1

The investigational products are Compound A and panitumumab. The title of study is “A First in Human, Phase 1a/1b, Open Label, Dose-Escalation and Expansion Study to Investigate the Safety, Pharmacokinetics and Antitumor Activities of the RAF Dimer Inhibitor Compound A in Patients with Advanced or Refractory Tumors.”

The study is a multicenter, open-label, 2-part (Safety Run-In and Dose Expansion) Phase 1a/1b study of Compound A in combination with panitumumab in patients with tumors harboring B-RAF or K-RAS/N-RAS mutations that may respond to a RAF dimer inhibitor.

The number of patients is approximately 64 patients in total. Part 1 (Safety Run-In) approximately 24 patients. Part 2 (Dose Expansion) has approximately 40 patients.

### Objectives and Endpoints

Study objectives for safety run-in include primary objectives, secondary objectives, and exploratory objectives. The Primary objectives are to assess the safety and tolerability of the combination of Compound A and panitumumab and to determine the recommended Phase 1b/2 dose for the combination. Secondary objectives are to characterize the pharmacokinetics (PK) of Compound A and panitumumab in combination and to assess the preliminary antitumor activity of the combination of Compound A and panitumumab. Exploratory objectives are to determine potential predictive biomarkers of efficacy, to assess potential pharmacodynamic (PD) biomarkers of target engagement, biological activity, and mechanism of action, and to explore mechanisms of treatment resistance in patients who fail to respond or develop resistance.

Study objectives for dose expansion include primary objectives, secondary objectives, and exploratory objectives. A primary objective is to assess the preliminary antitumor activity of the combination of Compound A and panitumumab. Secondary objectives are to further assess the safety and tolerability of the combination of Compound A and panitumumab and to further characterize the PK of Compound A and panitumumab. Exploratory objectives are to explore potential predictive biomarkers of efficacy, to assess potential PD biomarkers of target engagement, biological activity, and mechanism of action, and to explore mechanisms of resistance in patients who fail to respond or develop resistance.

### Study Endpoints for Safety Run-In

A primary endpoint is the safety and tolerability of the combination of Compound A and panitumumab assessed throughout the study by the incidence and severity of AEs and SAEs (coded to system organ class (SOC) and preferred term (PT) using the Medical Dictionary for Regulatory Activities (MedDRA)) and graded according to the Common Terminology Criteria for Adverse Events Version 5.0 (CTCAE v5.0), physical examination, ophthalmologic examination, vital signs, electrocardiograms (ECGs), echocardiograms (ECHOs), and laboratory tests. The recommended Phase 1b/2 dose is determined based on safety, tolerability, PK, preliminary efficacy, and other available data.

Secondary endpoints include determining a pharmacokinetic profile for Compound A and panitumumab in combination. This may include PK parameters including but not limited to single dose: area under the plasma concentration curve (AUC), maximum observed plasma concentration (Cₘₐₓ), time to maximum observed plasma concentration (Tₘₐₓ); steady state: AUC_{last,ss}, C_{max,ss}, and T_{max,ss}. This may further include efficacy parameters, including objective response rate (ORR), disease control rate (DCR), duration of response (DOR), clinical benefit rate (CBR), and progression-free survival (PFS). Exploratory Objectives are predictive biomarkers of efficacy, including but not limited to mitogen activated protein kinase (MAPK) signaling including phosphorylated extracellular signal-regulated kinase (phospho-ERK) level, v-RAF murine sarcoma viral oncogene homolog B (B-RAF), Kirsten rat sarcoma virus oncogene (K-RAS), neuroblastoma RAS viral oncogene (N-RAS), A-RAF proto-oncogene (A-RAF), neurofibromin-1 (NF-1) mutation, B-RAF or C-RAF amplification and other aberrations in, or affecting, the MAPK pathway.

### Study Endpoints for Dose Expansion

Primary endpoints are efficacy parameters, including: objective response rate (ORR), disease control rate (DCR), duration of response (DOR), clinical benefit rate (CBR), and progression-free survival (PFS). Secondary endpoints are: safety and tolerability assessment of AEs, physical examination, ophthalmologic examination, vital signs, ECG, ECHO, and laboratory measurements as described in Safety Run-In; PK parameters for Compound A, including but not limited to: AUC, Cₘₐₓ, and Tₘₐₓ; and PK parameters for panitumumab, including but not limited to: AUC, Cmax, and Tmax.

Exploratory endpoints are predictive biomarkers of efficacy, including but not limited to mitogen activated protein kinase (MAPK) signaling including phosphorylated extracellular signal-regulated kinase (phospho-ERK) level, v-RAF murine sarcoma viral oncogene homolog B (B-RAF), Kirsten rat sarcoma virus oncogene (K-RAS), neuroblastoma RAS viral oncogene (N-RAS), A-RAF proto-oncogene (A-RAF), neurofibromin-1 (NF-1) mutation, B-RAF or C-RAF amplification and other aberrations in, or affecting, the MAPK pathway.

### Study Design

This is a 2-part Phase 1b study of Compound A and panitumumab in combination in patients with tumors harboring oncogenic K-RAS/N-RAS or B-RAF mutations in colorectal cancer (CRC) and KRAS mutations in pancreatic cancer. Compound A in combination with panitumumab in CRC K-RAS/N-RAS or B-RAF nd pancreatic K-RAS mutated patients can have increased antitumor activities and address the high unmet medical needs in these patient populations.

The safety run-in is a multicenter, open-label, multiple-dose, dose-escalation study in patients with tumors harboring oncogenic B-RAF or K-RAS/N-RAS mutations in colorectal cancer or pancreatic cancer. Three combination dose levels are used. The Safety Monitoring Committee (SMC) evaluate safety data after patients have completed at least 1 cycle of treatment and decide upon subsequent dose levels. The number of pancreatic patients can not exceed one-third of the safety run-in cohort.

The dose expansion is a multicenter, open-label, multiple-group, noncomparative, indication expansion study for colorectal cancer with confirmed K-RAS/N-RAS mutations, with approximately 20 patients and pancreatic cancer with confirmed K-RAS mutations, with approximately 20 patients. Additional groups are added to the study as necessary to investigate emerging signals based on SMC advice and with agreement of the Institutional Review Board (IRB)/Independent Ethics Committee (IEC).

Patients are monitored for safety, tolerability, and efficacy throughout the study from the day of first administration of Investigational Medicinal Product (IMP) up to 30 days after the last administration of study drugs. Radiological assessment of tumor response is performed approximately every 6 (±1) weeks in the first six months every 8 (±1) weeks thereafter, after first year the assessment can be adjusted to every 12 (±1) weeks. Tumor response is assessed by investigators based on Response Evaluation Criteria in Solid Tumors (RECIST) 1.1.

Patients receive study drug(s) until progressive disease (PD), unacceptable toxicity, death, or another discontinuation criterion is met.

The study population is adult patients with advanced or metastatic, unresectable colorectal cancer or pancreatic cancer who have experienced disease progression during or after at least one line of systemic therapy or for which treatment is not available, not tolerated or refused. In the Safety Run-in, patients must have a known mutation status and a histologically or cytologically confirmed diagnosis of colorectal cancer or pancreatic cancer, harboring an oncogenic B-RAF or K-RAS/N-RAS mutation for which no effective standard therapy is available or acceptable to the patient. The number of pancreatic patients can not exceed one-third of the safety run-in cohort. In the Dose Expansion, patients must have a known mutation status and histologically or cytologically confirmed advanced or refractory solid colorectal cancer harboring an oncogenic K-RAS/N-RAS mutation or pancreatic cancer with K-RAS mutation for which no effective standard therapy is available or acceptable to the patient.

To be eligible for inclusion in the study, the patient must: be able to provide written informed consent and can understand and comply with the requirements of the study; be ≥18 years of age (or the legal age of consent in the jurisdiction in which the study is taking place) on the day of signing the Informed Consent Form (ICF); and have advanced or metastatic, unresectable colorectal cancer who have experienced disease progression per RECIST v1.1 during or after at least one line of systemic therapy or for which treatment is not available, not tolerated or refused.

In addition, patients must meet the following eligibility criteria for the corresponding part of the study. In the Safety Run-In, patients must have a known mutation status and a histologically or cytologically confirmed colorectal cancer or pancreatic cancer harboring an oncogenic B-RAF or K-RAS/N-RAS mutation for which no effective standard therapy is available or acceptable to the patient. The number of pancreatic patients can not exceed one-third of the safety run-in cohort. In the Dose Expansion, patients must have a known mutation status and histologically or cytologically confirmed advanced or refractory colorectal cancer harboring an oncogenic K-RAS/N-RAS mutation or pancreatic cancer with K-RAS mutations for which no effective standard therapy is available or acceptable to the patient. Eligible patients must further: have archival tumor tissue or agree to a tumor biopsy at baseline for mutation and biomarkers analysis; have measurable disease as defined per RECIST 1.1; have Eastern Cooperative Oncology Group (ECOG) performance status of ≤1 at screening; have life expectancy ≥12 weeks at the time of signing the ICF; have adequate organ function as indicated by the following laboratory values without transfusion within 14 days of the first dosing of IMP:
Absolute neutrophil count (ANC)≥1500 cells/μL
Platelets ≥100,000/μL
Hemoglobin ≥9 g/dL or ≥5.6 mmol/L
Serum creatinine ≤1.5× upper limit of normal (ULN) or calculated creatinine clearance >50 ml/min
Serum total bilirubin ≤1.5×ULN (total bilirubin must be <3×ULN for patients with Gilbert's syndrome) and
Aspartate aminotransferase (AST) and alanine aminotransferase (ALT)≤3×ULN or ≤5×ULN for patients with liver metastases.

Additionally, female patients are eligible to enter and participate in the study if they are of non-childbearing potential (ie, physiologically incapable of becoming pregnant), including any female who:has had a hysterectomy; has had a bilateral oophorectomy (ovariectomy); has had a bilateral tubal ligation; or is postmenopausal (total cessation of menses for ≥1 year). Female patients are also eligible to enter and participate in the study if they are of childbearing potential and have a negative serum pregnancy test within 7 days of the first dosing of IMP, are not breast feeding, and use protocol-approved contraception before study entry and throughout the study until 90 days after the last IMP administration. Male patients are eligible to enter and participate in the study if they are vasectomized or agree to use protocol-approved contraception during the study treatment period and for at least 90 days after the last dose of the IMP.

A patient may be excluded for prior treatment with any RAF or MEK inhibitor; current or history of central nervous system (CNS) metastasis; history or evidence of retinal pathology on ophthalmologic examination that is considered a risk factor for central serous retinopathy, RVO, or neovascular macular degeneration; having any of the following risk factors for RVO:
Intraocular pressure ≥21 mmHg
Grade ≥2 serum cholesterol
Grade ≥2 hypertriglyceridemia
Grade ≥2 or symptomatic hyperglycemia (fasting)
Grade ≥2 hypertension.

A patient may also be excluded if they: have history of glaucoma; have pulmonary fibrosis/interstitial lung disease (ILD); have an active parathyroid disorder or a history of malignancy associated hypercalcemia; have experienced any of the following within 6 months (24 weeks) of signing the informed consent form: clinically significant cardiac disease (New York Heart Association Class III or IV), myocardial infarction, severe/unstable angina, coronary/peripheral artery bypass graft, symptomatic congestive heart failure, cerebrovascular accident, transient ischemic attack, or symptomatic pulmonary embolism; have LVEF ≤50% as assessed by multigated acquisition (MUGA) scan or by ECHO; have abnormal QT interval corrected by Fridericia's formula (>450 msec for male patients, >470 msec for female patients, or >480 msec for patients with bundle branch block) after electrolytes have been corrected at screening; have current severe, uncontrolled systemic disease including, but not limited to, clinically significant cardiovascular, pulmonary, renal disease, or serious infection; have any unstable, preexisting major medical condition that in the opinion of the investigator contraindicates the use of an IMP, including known human immunodeficiency virus (HIV) or active hepatitis B virus (HBV) or hepatitis C virus (HCV) infection. Patients who are hepatitis B surface antigen (HBsAg) positive or HCV antibody positive at Screening are enrolled only if HBV DNA titers <500 IU/mL or negative HCV RNA polymerase chain reaction test, respectively.

A patient may also be excluded if they: have any hemorrhage or bleeding event at CTCAE v5.0 Grade 3 or higher within 28 days of Cycle 1 Day 1; have an increased serum calcium (>1×ULN) or serum phosphorus (>1×ULN) level; have an inability to swallow oral medications (capsules and tablets) without chewing, breaking, crushing, opening or otherwise altering the IMP formulation; have gastrointestinal illnesses (e.g., absorption syndrome); use concomitant systemic or ocular glucocorticoid therapy; use concomitant vitamin D; have a major surgical procedure or significant traumatic injury within 4 weeks prior to the first dose of IMP treatment in Cycle 1, or anticipate the need for major surgery during the course of study treatment; receive concomitant medicines that are strong CYP3A inhibitors; have a known history of significant toxicity from another RAF, MEK, ERK, or anti-EGFR antibody inhibitor requiring discontinuation of treatment from these drugs; had prior treatment of panitumumab and had dose reduction; have underlying medical conditions, laboratory abnormality, or alcohol or drug abuse or dependence that, in the investigator's opinion, are unfavorable for the administration of study drug or affect the explanation of drug toxicity or adverse events; have insufficient compliance during the study according to investigator's judgement; or are pregnant or nursing.

A patient may also be excluded for undergoing treatment with any of the following: cyclical chemotherapy within a period of time that was shorter than the cycle length used for that treatment (e.g., 6 weeks for nitrosourea, mitomycin-C) prior to starting study treatment; biologic therapy (e.g., antibodies) except bevacizumab or aflibercept, continuous or intermittent small molecule therapeutics, or any other investigational agents within a period of time that is ≤5 half-lives (t½) or ≤4 weeks (whichever is shorter) prior to starting study treatment; bevacizumab or aflibercept therapy ≤3 weeks prior to starting study treatment; and radiation therapy to >30% of the bone marrow at any time point or within 28 days prior to the first dose of study treatment.

The dose levels of panitumumab and Compound A in safety run-in are
Cohort 1: panitumumab at 6 mg/kg Q2W+Compound A at 10 mg QD
Cohort 2: panitumumab at 6 mg/kg Q2W+Compound A at 20 mg QD
Cohort 3: panitumumab at 6 mg/kg Q2W+Compound A at 40 mg QD

Compound A may also be administered at 5 mg, 15 mg, 25 mg, 30 mg, or 35 mg. The recommended dose of panitumumab is 6 mg/kg, administered as an intravenous infusion over 60 minutes, every 14 days. Doses higher than 1000 mg should be administered over 90 minutes. Compound A is administered orally (PO), once a day. The proposed dose levels may be modified; additional dose levels (including higher doses) or dosing regimens may be considered. Any modification of dose levels or dosing regimens are reviewed and approved by the SMC.

The dose level for dose expansion is Compound A administered orally (PO), once a day, continuously in 28-day cycles at a dose level determined by the Dose Escalation. In addition to daily oral dose of Compound A, patients receive panitumumab IV every 2 weeks in 28-day cycles at a dose level determined by the Dose Escalation.

The SMC makes a recommendation on the selection of the panitumumab dosing regimen based on available safety, efficacy, PK, and exploratory data from the Safety Run-In.

### Example 2

General properties of the solid form (Form F) of Compound A used are provided in Table 2.

TABLE 2
General Properties of the solid form (Form F) of Compound A used

| | |
|---|---|
| Properties | White to off-white solid |
| Solubility | The solubility of the sample is directly related to the pH of the |
| | solution, with a solubility of 0.24 mg/mL in 0.1N HCI and less than |
| | 5 μg/mL in both pH 4.5 buffer and pH 6.8 buffer. Surfactants |
| | improve the solubility to a large extent, especially sodium lauryl |
| | sulphate (SLS) and hexadecyl trimethyl ammonium bromide |
| | (HTAB), 1% SLS and 0.5% HTAB increased the solubility to 0.45 |
| | mg/mL and 0.31 mg/mL. At the same time, different concentrations |
| | of HTAB have different effects on solubility. |
| Hygroscopicity | At 80% relative humidity (RH), the sample had an actual moisture of |
| | 81.5% and a moisture absorption of about 5.425%; the sample was |
| | moderately hygroscopic. |
| Thermogravimetric | In the TGA and DSC spectra, the sample had 1.438% weight loss |
| Analysis (TGA) and | before 100° C. and the glass transition temperature was 111.07° C. |
| Differential Scanning | |
| Calorimetry (DSC) | |
| Crystal form | Under polarized light microscopy, samples were irregular shape. |
| | XRPD (XRPD): MBP Form F is an amorphous solid. |
| Particle size | D90 = 118 μm |
| distribution | |
| Flowability | Good |
| Stability | Form F is in amorphous form for 24 months at long-term condition |
| | (25° C./60% RH) as well as for 6 months at accelerated condition (40° C./ |
| | 75% RH) |
| Bulk density | 0.25 g/mL |
| Tap density | 0.42 g/mL |
| Solid form | Form F of Compound A in Example 7 of WO2020151756 |

Powder of Form F with particle sizes of D90<200 μm after micronization. The material content (purity) was not less than 98.0%.

### Example 3

The investigational products are Compound A and panitumumab. The title of the study is “A Phase 1b, Open-Label, Dose Escalation and Expansion Study to Investigate the Safety, Pharmacokinetics, and Antitumor Activity of Compound A with Panitumumab in Patients with Advanced or Metastatic RAS mutant Colorectal and Pancreatic Ductal Cancers.”

### Objectives and Endpoints

### Dose Finding (Part 1)

The primary objectives of Dose Finding (Part 1) are to assess the safety and tolerability of the combination of Compound A and panitumumab in participants with advanced or metastatic CRC with a known mutation status and tumor harboring an oncogenic mutation of BRAF, KRAS, or NRAS with documented disease progression during or after at least 1 line of prior therapy and to determine the MTD of Compound A in combination with panitumumab and the RP2D of the combination.

The secondary objectives of Dose Finding (Part 1) are to characterize the PK of Compound A and any relevant metabolites after single and multiple dose administration of the Compound A and panitumumab combination and to assess the preliminary antitumor activity of the combination of Compound A and panitumumab.

The primary endpoints of Dose Finding (Part 1) include: safety and tolerability of Compound A+panitumumab combination treatment is assessed by the incidence of SAEs and incidence and severity of all TEAEs and adverse events of special interest (AESI); the severity of all AEs is graded according to the National Cancer Institute Common Terminology Criteria for Adverse Events (NCI-CTCAE) v5.0; additional safety outcomes include:
Laboratory values (hematology, clinical chemistry, thyroid function testing, coagulation, and urinalysis).
Vital signs.
Electrocardiogram (ECG) and echocardiogram (ECHO)/multiple-gated acquisition (MUGA) findings (as applicable).
Physical and ophthalmological examinations.
Eastern Cooperative Oncology Group (ECOG) Performance status (PS).
Interruptions and/or reductions to dosing with Compound A.

The MTD is determined in accordance with the modified toxicity probability interval-2 (mTPI-2) design (Guo W, Wang S J, Yang S, Lynn H, Ji Y. A Bayesian interval dose-finding design addressing Ockham's razor: mTPI-2. Contemp Clin Trials. 2017 July; 58:23-33) and are based on the occurrence of DLTs, and the emerging safety, and tolerability profile of the Compound A+panitumumab combination.

The RP2D is based on safety, preliminary efficacy, and other complementary data from all dose finding cohorts.

The Sponsor makes a recommendation on the selection of the Compound A+panitumumab dose to be evaluated as the RP2D. Both the MTD and the RP2D must be confirmed by the SMC.

The secondary endpoints of Dose Finding (Part 1) are described here For Compound A and any relevant metabolites, the PK are assessed in plasma as delineated in the SOA (see Table 7). RECIST v1.1 is used to determine tumor response based on the following efficacy endpoints: ORR, defined as the proportion of participants treated with Compound A and panitumumab with confirmed CR or PR; duration of response (DOR), defined as the time from the first determination of response in those participants with a confirmed response until the first documentation of progression or death due to any cause, whichever occurs first; DCR, defined as the proportion of participants treated with Compound A and panitumumab with CR+PR+SD≥24 weeks; PFS, defined as the time from the date of the first administration of study drugs to the date of the first documentation of disease progression or death due to any cause, whichever occurs first.

### Dose Expansion (Part 2):

The primary objective of Dose Expansion (Part 2) is to determine the ORR as assessed by initial investigator review using RECIST v1.1 with Compound A and panitumumab combination treatment at the RP2D.

The secondary objectives of Dose Expansion (Part 2) are to further assess the safety and tolerability of Compound A and panitumumab in advanced or metastatic CRC patients with KRAS or NRAS mutations and in advanced or metastatic PDAC patients with KRAS mutations, with documented disease progression during or after at least 1 line of prior therapy; to determine the ORR as assessed by central review using RECIST v1.1 with Compound A and panitumumab combination treatment at the RP2D; to determine the preliminary activity of Compound A and panitumumab at the RP2D as assessed by DCR by initial investigator review using RECIST v1.1; DOR; and PFS; to further characterize the PK of Compound A and any relevant metabolites.

The exploratory objectives of Dose Expansion (Part 2), whose results may be reported separately to the final clinical study report (CSR), are to determine potential predictive biomarkers of molecular response to Compound A and panitumumab and to assess potential pharmacodynamic (PDx) biomarkers of target engagement, biological activity, and mechanism of action.

### Secondary Endpoints

A secondary Endpoint of Dose Expansion (Part 2) is safety and tolerability of Compound A and panitumumab combination treatment at the RP2D based on the incidence of SAEs, and incidence and severity of all TEAEs and AESI where the severity of all AEs is graded according to the NCI-CTCAE v5.0. Additional safety outcomes include: laboratory values (hematology, clinical chemistry, coagulation, and urinalysis); vital signs; ECG and ECHO/MUGA findings (as applicable); physical and ophthalmological examinations; ECOG PS; interruptions and/or reductions to dosing with Compound A; ORR, defined as the proportion of participants treated with Compound A and panitumumab at the RP2D with confirmed CR or PR; DCR, DOR and PFS—as defined for the Part 1 dose finding portion; determination of plasma concentrations of Compound A and any relevant metabolites.

### Exploratory Endpoints

Exploratory endpoints of Dose Expansion (Part 2) are predictive and PDx biomarkers, including but not limited to markers for mutation, amplification, transcription, and phosphorylation profiles of MAPK pathway signaling, assessed in the baseline tumor tissue and peripheral blood samples and exploratory subgroup analyses, e.g., of circulating tumor deoxyribonucleic acid (ctDNA)/serum, which may be performed where data permit, to investigate mechanisms of resistance in participants treated with the Compound A and panitumumab combination who fail to respond or develop resistance.

### Study Plan

This is a multicenter, global, open-label, Phase 1b dose finding and dose expansion study in participants with advanced or metastatic CRC and PDAC with tumors harboring oncogenic mutations of BRAF and KRAS/NRAS that may be responsive to treatment with Compound A, a RAF dimer inhibitor, given in combination with the EGFR inhibitor, panitumumab.

Participants are recruited to this study after initial referral from participating oncology centers and following at least 1 standard of care anticancer therapy. Eligible, consenting study participants is accrued sequentially to a dose finding (Part 1) phase, followed by a dose expansion (Part 2) phase. Part 1 of the study seeks to establish the MTD and RP2D by assessing the safety, tolerability, preliminary antitumor activity, and PK for the combination of Compound A with panitumumab. Part 2 of the study further evaluates the safety, PK, and assess the preliminary antitumor activity of the RP2D of the Compound A+panitumumab combination.

Exploratory investigation of predictive and PDx biomarkers is undertaken for both Part 1 and Part 2 of the study.

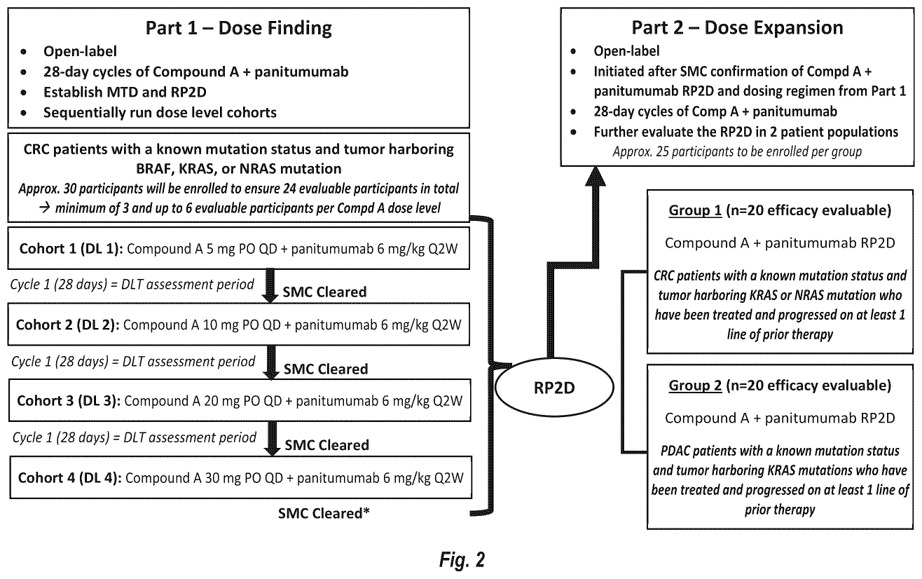

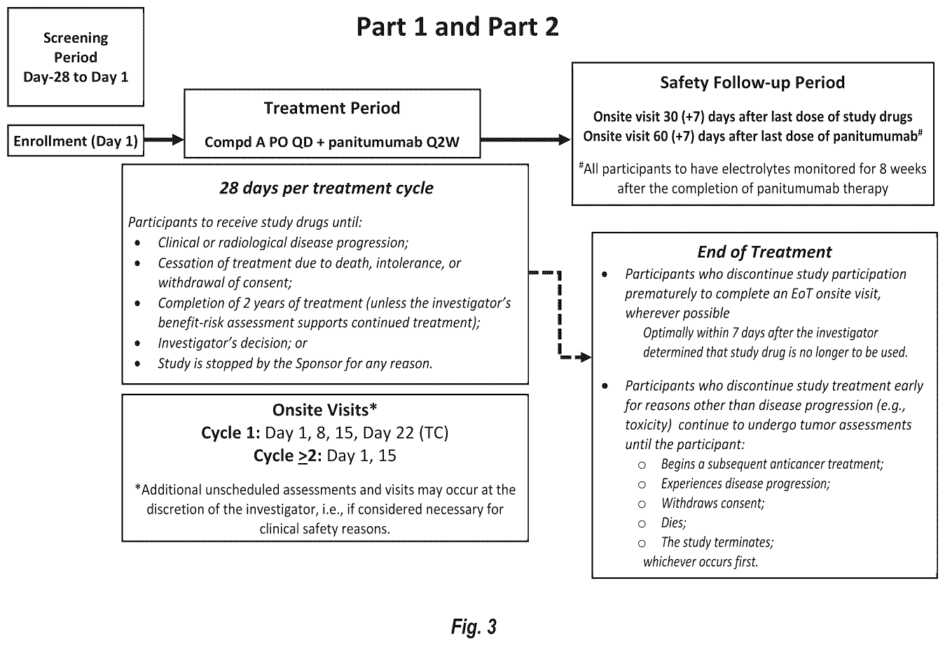

The study design is summarized in FIG. 2 (by study part) and FIG. 3 (by study period).

### Part 1—Dose Finding

Enrollment to the dose finding portion of the study occurs in 4 planned sequentially run cohorts consisting of a minimum of 3 and up to 6 evaluable participants per Compound A dose level given in combination with panitumumab. The dose finding cohorts enroll participants with advanced or metastatic CRC with a known mutation status and tumor harboring an oncogenic mutation of BRAF, KRAS, or NRAS and with documented disease progression by RECIST criteria during or after at least 1 line of prior therapy. Approximately 30 participants are enrolled to ensure approximately 24 evaluable participants in total.

All participants undergo repeated Compound A+panitumumab treatment cycles of 28 days. The starting dose of Compound A is 5 mg administered PO QD. Panitumumab is administered Q2W by IV infusion at 6 mg/kg of bodyweight and on these days is given within 60 minutes (+30 minutes) following oral dosing with Compound A.

The following dose levels for evaluation of the combination are used in Part 1:
Cohort 1 (dose level 1): Compound A 5 mg PO QD+panitumumab 6 mg/kg Q2W
Cohort 2 (dose level 2): Compound A 10 mg PO QD+panitumumab 6 mg/kg Q2W
Cohort 3 (dose level 3): Compound A 20 mg PO QD+panitumumab 6 mg/kg Q2W
Cohort 4 (dose level 4): Compound A 30 mg PO QD+panitumumab 6 mg/kg Q2W

### Dose Escalation and Cohort Progression

Periodic safety evaluation during the Part 1 dose finding portion of the study is performed by the SMC who convenes to actively monitor and review all cumulative data, including safety and efficacy data and all available PK and PDx data for the ongoing study during the dose finding portion. The SMC can make recommendations about early study closure or changes to the conduct of the study. Dose cohort size management and dose escalation decisions for Part 1 are made in accordance with the mTPI-2 model-assisted design (Guo et al., 2017) and must be confirmed by the SMC. The Sponsor may decide to stop or make adaptations to the study based upon the SMC recommendations.

The target toxicity rate for the MTD is ϕ=0.30 and the acceptable toxicity probability interval is (0.25, 0.33). The mTPI-2 design uses a Bayesian statistics framework and a beta-binomial hierarchical model to compute the posterior of dosing intervals that reflect the relative difference between the toxicity rate of each dose level. Unit Probability Mass (UPM) for each of the following toxicity probability intervals is calculated: (0, 0.01), (0.01, 0.09), (0.09, 0.17), (0.17, 0.25), (0.25, 0.33), (0.33,0.41), (0.41, 0.49), . . . , (0.89, 0.97), (0.97, 1).

Note that the width of each interval is 0.08 except for the first and last intervals. The UPM of an interval is the posterior toxicity probability of being in the interval divided by the width of the interval (Ji et al., 2010).

The recommendation according to the mTPI-2 design is as follows: escalate to the next dose (or stay at the same dose if the current dose is the highest dose level) if the maximum UPM is at an interval below (0.25, 0.33); stay at the same dose if the maximum UPM is within (0.25, 0.33); de-escalate to the last safe dose if the maximum UPM is at an interval above (0.25, 0.33). The following 2 additional safety rules (per Ji et al., 2010) are observed for this study:

Safety Rule 1 (early termination): Suppose that dose 1 has been used to treat participants. If the posterior toxicity probability being greater than 0.30 is more than 95%, terminate the trial due to excessive toxicity.

Safety Rule 2 (dose exclusion): Suppose the decision is to escalate from the current dose level I to the next level (i+1). If the posterior probability in dose level 1 being greater than 0.3 is more than 95% then treat the next cohort of participants at dose i and exclude doses (i+1) and higher from the study, that is, these doses are never used again in the study.

Evaluation of a cohort of at least 3 participants who have completed 1 cycle of treatment (up to and including Day 28) per Compound A dose level in combination with panitumumab is required prior to determining the dose level for the next dose finding cohort.

Table 3 shows the decision table based on mTPI-2 for each scenario of observed number of DLTs out of ‘n’ participants at a dose level that is used to assist the SMC in decision making. The SMC's decision to stay, escalate, or de-escalate a particular Compound A dose level also considers the totality of safety and efficacy data and all available PK and PDx data.

If the highest planned dose level (dose level 4—Compound A 30 mg PO QD+panitumumab 6 mg/kg Q2W) is cleared by the SMC based on the mTPI-2 estimate of the toxicity rate and the review of all available data in the study, then the Sponsor, in consultation with the SMC, may recommend escalation of Compound A to 40 mg (the MTD based on Compound A monotherapy) and/or additional dosing schedules. The highest dose of Compound A in the dose finding portion should not exceed 40 mg QD.

Cohorts may receive a Compound A dose level that has already been tested but a dose that is associated with a decision of “dose de-escalate, unacceptable toxicity” cannot be revisited and no additional participants should be treated at this dose or higher doses of Compound A for the remainder of the trial.

TABLE 3
Decision Table for Compound A-EGFR-001
Part 1-- Dose Finding using mTPI-2

| # DLT | N = 3 | N = 4 | N = 5 | N = 6 |
|---|---|---|---|---|
| 0 | E | E | E | E |
| 1 | D* | S | E | E |
| 2 | D | D | D | D |
| 3 | DU | DU | D | D |
| 4 | | DU | DU | DU |
| 5 | | | DU | DU |
| 6 | | | | DU |

E: Escalate to the next higher dose;
S: Stay at the same dose;
D: De-escalate to the previous lower dose;
DU: De-escalate to the previous lower dose and never use the current dose again in the study
*For participants in Dose Level 1, an additional participant is enrolled for evaluation.
Beta prior parameter: (1, 1), target toxicity probability posterior toxicity (pt) = 0.30, toxicity equivalence interval = (0.25, 0.33);
N = Total number of participants dosed at a given level;
# DLT = Total number of participants who experience dose limiting toxicities

### Dose Limiting Toxicities

For the purposes of tolerability decisions in Part 1, a DLT is defined as any AE or abnormal laboratory value assessed as unrelated to underlying disease, disease progression, intercurrent illness, or concomitant medications/therapies that occurs within the first 28-day cycle of treatment. The severity of DLTs is graded according to the NCI-CTCAE v5.0. A DLT must meet at least 1 of the following criteria:

A hematologic DLT is: any Grade ≥4 hematological toxicity; Febrile neutropenia (defined as absolute neutrophil count (ANC)<1000/mm³ with a single temperature of >38.3° C. (101° F.) or a sustained temperature of ≥38.0° C. (100.4° F.) for >1 hour); Grade 3 neutropenia lasting >7 days; Grade 3 thrombocytopenia with clinically significant (CS) bleeding; or Grade 3 anemia requiring transfusions per local or international guidelines in the absence of bleeding.

A nonhematologic DLT is: any death not clearly due to the underlying disease or extraneous causes and for any toxicity requiring permanent discontinuation of study drug(s); any Grade ≥4 nonhematologic event unless otherwise noted (see below); Grade ≥3 total bilirubin (TBIL; except in participants with Gilbert's Syndrome) or transaminases (ALT or AST); Grade ≥3 skin and subcutaneous tissue disorder that did not begin to resolve within 28 days despite initiating optimal medical and supportive care, and protocol-indicated dose management of Compound A and panitumumab; Grade ≥3 significant neurologic toxicity (e.g., seizure, hallucination, confusion, or delirium); Grade ≥3 creatine phosphokinase (CPK) increased with symptoms or Grade ≥2 rhabdomyolysis; Grade ≥3 nonhematologic treatment related toxicity not listed above that did not resolve to Grade ≤1 within 3 days of initiating optimal medical and supportive therapy; other clinically important or persistent toxicities may also be considered a DLT following review by the Sponsor in consultation with investigators or following review by the SMC; confirmed occurrence of Hy's law or potential drug-induced liver injury (DILI) per the US FDA definition, that cannot be explained by any other cause(s) (e.g., viral hepatitis, exposure to other hepatotoxins), without evidence of cholestasis and includes the following laboratory abnormalities (this specific category of DLT uses ULN rather than NCI-CTCAE grade for definition):
Participants with AST or ALT and TBIL baseline values within the normal range who subsequently present with AST or ALT values ≥3× upper limit of normal (ULN) concurrent with a TBIL value ≥2×ULN with no evidence of hemolysis and an ALP value ≤2×ULN or other findings indicating an absence of cholestasis.
For participants with baseline ALT or AST values above the ULN, AST or ALT value ≥2× the baseline values should be used in the definition mentioned above.

Additionally, any CS toxicities that occur after the defined DLT observation period for a given dose level may be considered for subsequent dose escalation and/or RP2D decisions. The following toxicities are not considered a DLT: isolated and asymptomatic Grade ≥3 laboratory abnormalities not listed above that have no clinical correlation and resolve to Grade ≤1 within 3 days with or without the initiation of medical and supportive therapy; Grade ≥3 panitumumab infusion reaction; Grade ≥3 laboratory abnormalities that do not appear clinically relevant or harmful and can be corrected (such as hypoalbuminemia and lymphopenia); Grade ≥3 nausea, vomiting, or diarrhea that resolves to Grade ≤1 within 3 days; Grade ≥3 fatigue that resolves to Grade ≤1 within 5 days; Grade ≥3 asymptomatic increase of lipase or amylase without pancreatitis that resolves to Grade ≤1 within 7 days.

Participants who experience a DLT are managed according to the guidelines for treatment modification due to TEAEs in this protocol.

To be considered DLT evaluable, a participant must have received ≥80% of the assigned doses of Compound A in combination with both doses of panitumumab and remained on study for 28 days from Cycle 1 Day 1 administration of Compound A+panitumumab. If a participant interrupts study drug for any reason other than a DLT and receives less than 80% of assigned doses, then that participant is replaced.

### Early Stopping Rules

In the dose finding portion, participants are carefully monitored for AEs, SAEs and DLTs. The recommendations according to the mTPI-2 model-assisted design. If the lowest dose is under investigation and the UPM is at an interval above (0.25, 0.33), then the trial is terminated due to excessive toxicity. Additionally, the study is terminated due to excessive toxicity using Safety Rule 1 if the posterior toxicity probability being greater than 0.30 is demonstrated to be more than 95% for the lowest dose. Otherwise, the study is continued based on the mTPI-2 recommendations.

### Maximum Tolerated Dose (MTD)

The MTD is determined in accordance with the mTPI-2 model-assisted design and is based on the occurrence of DLTs and the emerging safety and tolerability profile of the Compound A+panitumumab combination. Using the mTPI-2 model, the target toxicity rate for the MTD is ϕ=0.30 and the acceptable toxicity probability interval is (0.25, 0.33).

### Recommended Phase Two Dose (RP2D)

The RP2D is the dose level and dosing regimen for the Compound A+panitumumab combination chosen for further investigation in the dose expansion portion of the study. The Sponsor provides a recommendation on the selection of the Compound A+panitumumab dose to be evaluated. The RP2D must be confirmed by the SMC based on the safety, preliminary efficacy, and other complementary data from all dose finding cohorts.

### Part 2—Dose Expansion

The Part 2 dose expansion portion of the study is initiated only after confirmation of the RP2D and dosing regimen in Part 1 of the dose finding portion of the study. Participants enrolled in the dose finding cohorts may not be re-enrolled in the dose expansion portion of the study. The dose expansion portion of the study evaluates the RP2D in the following groups:

Group 1: Participants with advanced or metastatic CRC that harbors KRAS or NRAS mutations who have been treated and had documented disease progression by RECIST criteria during or after at least 1 line of prior therapy.

Group 2: Participants with advanced or metastatic PDAC that harbors KRAS mutations who have been treated and had documented disease progression by RECIST criteria during or after at least 1 line of prior therapy.

The expansion cohorts each enroll approximately 25 participants to achieve 20 efficacy evaluable participants per group. All participants undergo repeated Compound A+panitumumab treatment cycles of 28 days.

### Study Periods

Each study part is comprised of a screening, treatment, and safety follow-up period (FIG. 3). The screening, treatment, and follow-up schedules are the same for both Part 1 and Part 2 of the study, as delineated in Table 7.

### Screening Period

Screening for the study occurs within 28 days prior to study enrollment and the first administration of study drugs on Day 1 of treatment Cycle 1. Informed consent (from the participant or their legally authorized representative) must be documented before any study-specific procedures, including for screening, are performed.

Consenting participants who meet all the eligibility criteria at screening are enrolled upon confirmation of eligibility.

There is a minimum washout/recovery period for prior anticancer treatments (e.g., systemic chemotherapy, radiotherapy, biologic therapies, continuous or intermittent small-molecule therapies, or any other investigational agent) and major surgery before the start of study treatment, as defined in the study eligibility criteria.

Baseline assessments are conducted in all participants prior to administration of the first dose of the study drugs. Collection of tumor tissue at the screening visit, either from archived tumor tissue or fresh tumor biopsies, is mandatory to establish baseline retrospective mutation status.

Collection of fresh baseline tumor biopsies for analysis at screening is strongly recommended in participants with readily accessible tumor lesions. Participants are selected for screening and eligibility based on known mutation status from the local molecular testing result obtained from the tumor tissue sample collected any time prior to screening. Blood samples are collected from all participants at baseline for biomarker analysis.

All screening and baseline assessments are outlined in Table 7.

### Treatment Period

The treatment period for both the Part 1 dose finding and Part 2 dose expansion portions of the study is below.

Participants undergo repeated treatment cycles for 28 days with the Compound A+panitumumab combination. Participants receive study drugs until such time as: clinical or radiological disease progression; participants who are found to have clinical or radiological progressive disease (PD) by RECIST v1.1 are discontinued from the study; cessation of study treatment due to death, intolerance, or withdrawal of consent from the study; completion of 2 years of treatment (unless the investigator's benefit-risk assessment supports continued treatment); investigator's decision; or Study is stopped by the Sponsor for any reason.

All participants who discontinue study participation prematurely complete all specified assessments at an end of treatment (EoT) visit, wherever possible. The EoT visit is optimally conducted within 7 days after the investigator determines that study drug is no longer to be used.

Participants who discontinue study treatment early for reasons other than disease progression (e.g., toxicity) continue to undergo tumor assessments following the tumor response assessment schedule per protocol until the participant begins a subsequent anticancer treatment, experiences disease progression, withdraws consent, dies, or until the study terminates, whichever occurs first.

### Safety Follow-Up Period

The safety follow-up period consists of an onsite visit at 30 (+7) days after the last dose of the study drugs and another onsite visit at 60 (+7) days after the last dose of panitumumab.

After the defined safety follow-up period, investigators should continue to report any SAEs that are considered to be related to the study drug(s), as well as deaths, irrespective of the cause, if they become aware of them. Study participants who discontinue the study drugs due to a Grade ≥3 drug-related AE are followed until the resolution of the AE (to Grade ≤1, baseline, or stabilization) or initiation of a new anticancer treatment, whichever occurs first.

Additional unscheduled assessments and visits may occur at the discretion of the investigator, i.e., if considered necessary for clinical safety reasons.

### Population

### Definitions

Participants officially enter screening with informed consent.

Screen failures are defined as participants who consent to participate in the clinical study but are not subsequently enrolled to study intervention. A minimal set of screen failure information is required to ensure transparent reporting of screen failure participants to meet the Consolidated Standards of Reporting Trials (CONSORT) publishing requirements and to respond to queries from regulatory authorities. Minimal information includes demography, screen failure details, eligibility criteria, and any SAEs.

Individuals who do not meet the criteria for participation in this study (screen failure) for administrative reasons, or who have borderline test results, may be rescreened once. Rescreened participants should repeat all abnormal screening test and procedures.

Eligible, consenting participants are enrolled to the study. Prior to enrollment of a participant, the following must occur:

Confirmation that the participant (or LAR) has voluntarily signed the ICF.

Enrollment occurs only if the participant meets all study eligibility criteria and has been assessed by the investigator as being an appropriate candidate for study participation.

Prospective approval of protocol deviation to recruitment and enrollment criteria, also known as protocol waivers or exemptions, are not permitted.

To assess any potential impact on participant eligibility with regard to safety, the investigator must refer to the Compound A IB for detailed information regarding warnings, precautions, contraindications, AEs, and other significant data pertaining to the study drugs used in this study.

### Inclusion Criteria

Participants meeting all the following inclusion criteria at screening (and on day of first dosing, where applicable) are eligible for participation in the study:

Participants (or LAR) have voluntarily agreed to study participation by giving written informed consent and must be ≥18 years of age on the day of signing the ICF.

Participants with histologically confirmed advanced or metastatic solid tumors who have had documented disease progression by RECIST criteria during or after at least 1 prior line of systemic anticancer therapies in the representative population or are unable to receive standard of care therapy(ies) as noted by local guidelines.

Participants must meet the following eligibility criteria for the corresponding portion of the study:

Dose Finding: Participants with CRC with a known mutation status by local testing and tumor harboring an oncogenic mutation of BRAF, KRAS, or NRAS in the archival tumor sample or fresh tumor biopsy.

Dose Expansion: Participants must have a known mutation status by local testing and meet one of the following criteria according to the cohort they are enrolled into:

Group 1: Participants with CRC that harbors KRAS or NRAS mutations in the archival tumor sample or fresh tumor biopsy.

Group 2: Participants with PDAC that harbors KRAS mutations in the archival tumor sample or fresh tumor biopsy.

Participants must provide archival tumor tissue or a fresh tumor biopsy for retrospective mutation status analysis by Clinical Laboratory Improvement Amendments (CLIA)-certified next generation sequencing (NGS) assay and for biomarker analysis. Fresh tumor biopsies are strongly recommended at screening in participants with readily accessible tumor lesions.

Participants must have: radiologically measurable disease as defined per RECIST v1.1 at screening; ECOG PS of ≤1 at screening; life expectancy ≥12 weeks at screening according to investigator's best judgment; baseline serum electrolytes must be within the normal range per local laboratories, where for baseline serum electrolytes that are out of range, these may be corrected, and the potential participant may be rescreened; Adequate renal function characterized by estimated creatinine clearance; ≥50 mL/min Chronic Kidney Disease Epidemiology Collaboration equation (CKD-EPI); TBIL ≤1.5×ULN (≤3×ULN for participants with Gilbert's syndrome); AST and ALT ≤3×ULN or ≤5×ULN for participants with liver metastases; Adequate cardiac function, as determined by:
Systolic blood pressure <160 mmHg and diastolic blood pressure <100 mmHg,
(Grade ≤2) despite optimal antihypertensive management,
Left ventricular ejection fraction (LVEF) ≥50% by ECHO or MUGA,
No CS ECG waveform abnormalities,
QTcF≤470 msec, as determined by the mean QTcF values from the ECG assessments at screening (one triplicate),

And adequate hematologic and organ function, as indicated by the following laboratory values, prior to Cycle 1 Day 1: ANC 21500/mm³, or 1.5×10⁹/L; Platelet count ≥100,000/mm³, or 1.0×10⁹/L; Hemoglobin ≥8 g/dL, where for hematologic function, participant must not have received blood transfusion or growth factor support ≤14 days before the sample collection. Female participants are eligible to enter and participate in the study if they are of: nonchildbearing potential; with a negative serum pregnancy test at screening (within 7 days of the first dosing of study drugs) and agree to use contraception before study entry and throughout the study until 180 days after the last administered dose of the study drugs. Male participants are eligible to enter and participate in the study if they are vasectomized or agree to the use of contraception during the study treatment period and for at least 180 days after the last administered dose of the study drugs.

Participants meeting any of the following exclusion criteria at screening and on the day of first dosing are not eligible for the study: female participants who are pregnant or lactating; any major surgery within 4 weeks prior to Cycle 1 Day 1; active infection requiring systemic treatment at the start of the study treatment; participants receiving cancer therapy (chemotherapy or other systemic anticancer therapies, immunotherapy, radiation therapy, or surgery) at the time of Cycle 1 Day 1. Examples of such cancer therapies are: systemic chemotherapy within 4 weeks, or nitrosourea and mitomycin C within 6 weeks prior to Cycle 1 Day 1; biologic therapy (i.e., antibodies), continuous or intermittent small-molecule therapies, or any other investigational agents within a period of 5 times the half-life of the agent or within 4 weeks (whichever is shorter) prior to Cycle 1 Day 1; and curative radiation therapy within 2 weeks of Cycle 1 Day 1.

Participants meeting any of the following exclusion criteria at screening and on the day of first dosing are also not eligible for the study: any of the following cardiovascular criteria: current evidence of unstable angina or another form of symptomatic cardiac ischemia; symptomatic pulmonary embolism or other CS episode of thromboembolic disease ≤6 months before Cycle 1 Day 1; acute myocardial infarction ≤6 months prior to Cycle 1 Day 1; heart failure of New York Heart Association Classification III or IV ≤6 months prior to Cycle 1 Day 1; Grade ≥2 ventricular arrhythmia <6 months prior to Cycle 1 Day 1; cerebrovascular accident (CVA) or Grade ≥2 transient ischemic attack (TIA)≤6 months prior to Cycle 1 Day 1; Grade ≥2 hypertension that cannot be managed by standard anti-hypertension medications before Cycle 1 Day 1.

Participants meeting any of the following exclusion criteria at screening and on the day of first dosing are also not eligible for the study: Syncope or seizure ≤6 months before Cycle 1 Day 1; Any major surgery within 28 days prior to Cycle 1 Day 1; participants with toxicities that have not recovered to Grade ≤1 or stabilized and those Grade 2 toxicities listed as permitted in other eligibility criteria; Grade 2 neuropathy or isolated asymptomatic Grade 2 laboratory abnormality without a clinical correlate may be acceptable at the investigator's discretion after consultation with the medical monitor; Participants with a history of pneumonitis or interstitial lung disease; Participants with immune-related toxicities including myositis, dermatological toxicities, colitis, and myocarditis that have not resolved with appropriate management (i.e., thyroid replacement or diabetes management) following discontinuation of checkpoint inhibitor use; history or presence of gastrointestinal disease or other condition known to interfere with the absorption of drugs; history of ulcerative colitis or Crohn's disease or protracted and ongoing immune-mediated diarrhea from prior checkpoint inhibitor use; history of corneal perforation, keratitis, or severe dry eye; current evidence of symptomatic CNS metastases, leptomeningeal carcinomatosis, or untreated spinal cord compression. Asymptomatic treated or asymptomatic untreated brain metastases are allowed if participants are clinically stable in the judgment of the investigator; any active malignancy ≤3 years before Cycle 1 Day 1 except for the specific cancer under investigation in this study and any localized or noninvasive cancer that has been treated curatively (e.g., resected basal or squamous cell skin cancer, superficial bladder cancer, or carcinoma in situ of the cervix or breast); Any unstable, preexisting major medical condition that in the opinion of the investigator contraindicates the use of the study drugs, including known human immunodeficiency virus (HIV) or active hepatitis B virus (HBV) or hepatitis C virus (HCV) infection; participants who are hepatitis B surface antigen (HBsAg) positive or HCV antibody positive at screening may be enrolled only if HBV deoxyribonucleic acid (DNA) titers <500 IU/mL or negative HCV ribonucleic acid (RNA) polymerase chain reaction test, respectively; participants who have HIV infection at screening may be enrolled only if CD4+ T-cell (CD4+) counts ≥350 cells/μL; known hypersensitivity to RAF inhibitors, anti-EGFR monoclonal antibodies, or their excipients; any known history of Grade ≥3 toxicity lasting >14 days from another RAF, MEK, ERK, or anti-EGFR antibody inhibitor requiring discontinuation of treatment from these drugs; history of solid organ transplant requiring anti-rejection drugs; psychological, familial, sociological, or geographical conditions that are likely to interfere with compliance to the protocol; unfit for the study, in the opinion of the investigator, after medical interview, physical examination, and/or screening investigations; receiving any of the prohibited medications listed in the protocol or are anticipated to require any of these medications; concurrent participation in another therapeutic clinical trial; receiving any treatment with a strong CYP3A4 inhibitor or inducer ≤14 days (or 5 half-lives, whichever is longer) before Cycle 1 Day 1 and until completion of dosing with Compound A for at least 5 half-lives.

### Study Restrictions

### Contraceptive Requirements

Any participant who becomes pregnant during the study must immediately discontinue further treatment with study drug.

### Fasting and Dietary Restrictions

Participants are not required to fast prior to collection of blood samples for safety laboratory testing.

For both Part 1 and Part 2, Compound A is administered orally 1 hour before or 2 hours after a meal on the morning of each dosing day during the treatment period.

Consumption of grapefruit and grapefruit juice, Seville oranges, pomelos, exotic citrus fruits or grapefruit hybrids are not allowed within 14 days before the start of study treatments, during the treatment period, and until the EoT visit. No other dietary restrictions apply.

### Other Lifestyle Restrictions

Exposure to sunlight can exacerbate dermatologic toxicity associated with Compound A and panitumumab treatment. Participants are advised to wear sunscreen and hats and limit sun exposure while receiving treatment with the study drugs.

### Prior and Concomitant Therapies

Cancer therapy or procedures (chemotherapy or other systemic anticancer therapies, immunotherapy, radiation therapy, or surgery) are prohibited or restricted during the study at the time of Cycle 1 Day 1 and until completion of the study treatment period, such therapies or procedures are as follows: systemic chemotherapy within 4 weeks of the last dose or 6 weeks for nitrosourea and mitomycin C prior to Cycle 1 Day 1 until completion of the treatment period; biologic therapy (i.e., antibodies), continuous or intermittent small-molecule therapies, or any other investigational agents within a period of 5 times the half-life of the agent or within 4 weeks (whichever is shorter) prior to Cycle 1 Day 1 until completion of the treatment period; any major surgery within 4 weeks prior to Cycle 1 Day 1 until completion of the treatment period.

The following therapies are prohibited or restricted during the study: any major surgery within 4 weeks prior to Cycle 1 Day 1 until completion of the treatment period; chronic systemic or ocular glucocorticoid therapy within the 14 days before the start of study treatments and until completion of the treatment period with the following exceptions: physiologic or stress doses of steroids, when indicated in participants with endocrine deficiencies, corticosteroids given as the premedication for blood product transfusions, corticosteroids given as the pulse treatment for an acute allergic reaction or bronchospasm, inhaled corticosteroids for asthma and reactive airway disease, oral or IV corticosteroids for up to 6 consecutive days, when indicated for the management of an AE (e.g., Grade ≥3 thrombocytopenia) after consultation with the medical monitor and/or ophthalmologist (as relevant).

The following therapies are prohibited or restricted during the study: platelet or blood transfusion for the treatment of thrombocytopenia within 14 days before the start of study treatments and during the DLT period (Cycle 1), though platelet or blood transfusion for the treatment of thrombocytopenia may be permitted at the investigator's discretion after that particular event of thrombocytopenia has been identified as a DLT; red blood cell (RBC) or blood transfusion or erythropoietin (EPO) for the treatment of anemia within 14 days before the start of study treatments and during the DLT assessment period (Cycle 1), though RBC or blood transfusion for the treatment of anemia may be permitted at the investigator's discretion if the chronic anemia has been persistent within 28 days before the start of study treatments; granulocyte-colony stimulating factor (GCSF)/granulocyte-macrophage colony stimulating factor (GMCSF) for the treatment of leukopenia/neutropenia within 14 days before the start of study treatments and during the DLT assessment period (Cycle 1); strong CYP3A4 inhibitors and inducers (see Table 4) within 14 days before the start of study treatments and during the treatment period until completion of the treatment period; and substrates of CYP2C8 or CYP2C9 with a narrow therapeutic range (see Table 5) during the treatment period until completion.

EPO for the treatment of anemia may be permitted at the investigators discretion if the EPO dose has been stable within 28 days before the start of study treatments.

The following therapies are also prohibited or restricted during the study: Herbal remedies with a known potential to interfere with liver or other major organ functions (i.e., hypericin) or those which act as a strong CYP3A4 inhibitor, within 14 days before the start of study treatments until completion of the treatment period; live and live attenuated vaccines (within 4 weeks before the start of the study treatments), including the intranasal flu vaccine and the monkeypox vaccine, are prohibited; and Coronavirus disease 2019 (COVID-19) vaccine procedure must be completed at least 2 weeks before the start of the study treatments, COVID-19 vaccination is prohibited during the DLT period (Cycle 1) but is permitted from Day 1 of Cycle 2 until the end of the study, though a brief treatment hold (1-2 days before and/or after each dose of the vaccine) may be indicated at the investigator's discretion. Note: Injectable flu vaccine is permitted at any time before and during the trial.

COVID-19 therapy using PAXLOVID (nirmatrelvir+ritonavir) is contraindicated with drugs that are highly dependent upon CYP3A for clearance; as in vitro data has shown Compound A is mainly metabolized by the CYP3A pathway.

The following radiation therapies are prohibited or restricted during the study: prior curative radiotherapy is prohibited within 2 weeks of Cycle 1 Day 1; concomitant radiotherapy to tumor lesion(s) chosen as target lesions is prohibited from the start of study treatments and until completion of the treatment period, prior and concomitant palliative radiotherapy to non-target tumor lesions may be allowed at the investigator's discretion at any time before the start of study treatments and during the treatment period.

TABLE 4
Strong CYP3A4 Inhibitors and Inducers

| | | |
|---|---|---|
| CYP3A4 | Strong | boceprevir, cobicistat, danoprevir and ritonavir, elvitegravir and |
| | Inhibitors | ritonavir, nirmatrelvir and ritonavir, grapefruit juice, indinavir |
| | | and ritonavir, itraconazole, ketoconazole, lopinavir and |
| | | ritonavir, paritaprevir and ritonavir and ombitasvir and/or |
| | | dasabuvir, posaconazole, ritonavir, saquinavir and ritonavir, |
| | | telaprevir, tipranavir and ritonavir, telithromycin, |
| | | troleandomycin, voriconazole, clarithromycin, idelalisib, |
| | | nefazodone, nelfinavir |
| | Strong | apalutamide, carbamazepine, enzalutamide, mitotane, |
| | Inducers | phenytoin, rifampin, St. John's wort |

Abbreviations: CYP3A4 = cytochrome P450, family 3, subfamily A, member 4.

The list of drugs presented is not exhaustive. Please refer to the prescribing information for the concomitant medication to check for CYP3A4 inhibition/induction risks or contact the study medical monitor.

TABLE 5
CYP2C8 and CYP2C9 Substrates

| CYP2C8 Substrates | CYP2C9 Substrates | |
|---|---|---|
| amodiaquine, cerivastatin, | NSAIDs: | diclofenac, ibuprofen, lornoxicam, |
| paclitaxel, *repaglinide, | | meloxicam, S-naproxen, piroxicam, |
| sorafenib, torsemide | | suprofen |
| | Oral Hypoglycemic Agents: | tolbutamide, glipizide |
| | Angiotensin II Blockers: | losartan, irbesartan |
| | Sulfonylureas: | glyburide, glibenclamide, glipizide, |
| | | glimepiride, tolbutamide |
| | Others: | amitriptyline, celecoxib, fluoxetine, |
| | | Fluvastatin, glyburide, nateglinide, |
| | | phenytoin, **rosiglitazone, |
| | | tamoxifen, torsemide, valproic acid, |
| | | warfarin, |
| | | **zakirlukast |

Abbreviations: CYP2C8 = cytochrome P450, family 2, subfamily C8; CYP2C9 = cytochrome P450, family 2, subfamily C94; NSAID = nonsteroidal anti-inflammatory drug.
*CYP2C8 substrates with narrow therapeutic range.
**CYP2C9 substrates with narrow therapeutic range.

The list of drugs presented is not exhaustive. Please refer to the prescribing information for the concomitant medication to check for CYP2C8 or CYP2C9 substrates or contact the study medical monitor.

### Study Duration and Length of Participation

Each study part consists of a screening, treatment, and safety follow-up period. The screening, treatment, and follow-up schedules are the same for both parts of the study, as delineated in the SOA (Table 1). Screening Period represents the period (up to 28 days) between the date of signing the informed consent until the first administration of study drugs. Treatment Period for both the dose finding and dose expansion portions of the study is as follows:
Participants undergo repeated treatment cycles for 28 days with the Compound A+panitumumab combination.
Participants receive study drugs until such time as:
Clinical or radiological disease progression.
Participants who are found to have clinical or radiological progressive disease (PD) by RECIST v1.1 are discontinued from the study.
Cessation of study treatment due to death, intolerance, or withdrawal of consent from the study.
Completion of 2 years of treatment (unless the investigator's benefit-risk assessment supports continued treatment).
Investigator's decision; or
Study is stopped by the Sponsor for any reason.

All participants who discontinue study participation prematurely complete all specified assessments at an end of treatment (EoT) visit, wherever possible. The EoT visit is optimally conducted within 7 days after the investigator determines that study drug is no longer to be used.

Participants who discontinue study treatment early for reasons other than disease progression (e.g., toxicity) continue to undergo tumor assessments following the tumor response assessment schedule per protocol until the participant begins a subsequent anticancer treatment, experiences disease progression, withdraws consent, dies, or until the study terminates, whichever occurs first.

Safety Follow-up Period consists of a follow-up onsite visit at 30 (+7) days after the last dose of the study drugs and another visit at 60 (+7) days after the last dose of panitumumab.

Note: After the defined safety follow-up period, investigators should continue to report any SAEs that are considered to be related to the study drug(s), as well as deaths, irrespective of the cause, if they become aware of them. Study participants who discontinue the study drugs due to a Grade ≥3 drug related AE are to be followed until the resolution of the AE (to Grade ≤1, baseline, or stabilization) or initiation of a new anticancer treatment, whichever occurs first.

Additional unscheduled assessments and visits may occur at the discretion of the investigator, i.e., if considered necessary for clinical safety reasons.

### Investigational Product, Dosage and Mode of Administration

Study drugs include Compound A and panitumumab.

All participants undergo repeated Compound A+panitumumab treatment cycles of 28 days.

Two (2) weeks prior to starting study treatment, during the screening period, prophylactic management should be started including moisturizers, sun protection, an oral antibiotic, and a topical steroid.

### Compound A

Compound A is supplied as an oral, solid immediate-release capsule formulation in strengths of 5 mg and 10 mg.

The Compound A capsules are packaged in high density polyethylene (HDPE) bottles and should be stored according to the conditions specified on the label. Storage conditions for Compound A are supported by stability data conducted on the drug product. The starting dose of Compound A in the dose finding portion of the study is 5 mg administered orally QD. The highest doses of Compound A do not exceed 40 mg QD. Compound A is administered orally 1 hour before or 2 hours after a meal on the morning of each day during the treatment period.

### Panitumumab

Panitumumab (Vectibix—Amgen Inc.) is supplied as sterile, single use vials and administered Q2W by IV infusion via an infusion pump at 6 mg/kg of bodyweight. Prior to infusion, panitumumab should be diluted in a sodium chloride 9 mg/mL (0.9%) solution for injection to a final concentration not to exceed 10 mg/mL. Panitumumab must be administered using a low protein binding 0.2 μm or 0.22 μm in-line filter, through a peripheral line or indwelling catheter. The recommended infusion time is approximately 60 minutes. If the first infusion is tolerated, then subsequent infusions may be administered over 30 to 60 minutes. Doses higher than 1000 mg should be infused over approximately 90 minutes. A reduction in the rate of infusion may be necessary in cases of infusion-related reactions.

Infusion of panitumumab should be started within 60 minutes (+30 minutes) following oral dosing with Compound A capsules. Refer to the panitumumab summary of product characteristics (SmPC) and United States (US) prescribing information (PI) for all preparation and administration instructions.

### Safety Assessments

### Physical Examination

Complete and abbreviated, symptom-directed physical examinations are performed by a licensed physician at the time points specified in the SOA (Table 7). A complete physical examination includes assessments of 1) head, eyes, ears, nose, throat 2) cardiovascular, 3) dermatological, 4) musculoskeletal, 5) respiratory, 6) gastrointestinal, and 7) neurological systems. Limited, symptom-directed examinations are performed at the specified time points or as clinically indicated. Abnormalities observed at baseline are recorded on the Medical History and Baseline Conditions eCRF. At subsequent visits, new or worsened, CS abnormalities are recorded on the AE eCRF.

Potential dermatological toxicity is assessed as part of the physical examination with the characteristics and grade of rash. Any abnormality identified at baseline is recorded on the Medical History eCRF with appropriate disease/condition terms. New or worsening CS abnormalities must be recorded as AEs in the eCRF.

Physical examinations may be performed at various unscheduled time points if deemed necessary by the investigator.

### Vital Signs

Vital signs include measurements of body temperature, heart rate, respiratory rate, and blood pressure (systolic and diastolic) after the participant has been sitting for at least 5 minutes. Pulse oximetry should also be performed and documented. Blood pressure and heart rate measurements are assessed with a completely automated device. Manual techniques are used only if an automated device is not available. Body temperature is measured using a tympanic thermometer.

### Electrocardiogram Monitoring

A 12-lead ECG is performed at screening and during the treatment period within 60 minutes pre-dose and at 2 to 4 hours after Compound A dosing on Cycle 1 Day 1, Cycle 1 Day 8, and Cycle 2 Day 1 (see Table 7). Multiple time point ECGs beyond Cycle 2 Day 1 are not required but may be obtained if clinically indicated. ECGs are required at EoT and in the safety follow-up period. To reduce spurious readings, every effort should be made to perform ECGs in triplicate, with 1- to 2-minute intervals between ECG readings; however, a single ECG is allowed, if necessary, i.e., due to a public health emergency. ECGs are performed with participants in a supine or semi-reclined position for at least 5 minutes before the reading is taken. Heart rate, PR, QRS, QTcF, RR and result interpretation are recorded. Additional ECG monitoring may be performed at other times if deemed necessary by the investigator. When ECG assessment coincides with any other study procedures at the same time point, the ECG must be performed first, followed by vital signs, and then blood sample collection, with blood sample collected at the nominal time.

For safety monitoring purposes, the investigator or designee must review, sign, and date all ECG tracings. The investigator or designee evaluates the overall ECG result as normal, or abnormal not clinically significant (NCS), or abnormal CS. All abnormal CS ECG results must be reported as an AE or SAE. Paper or electronic copies of ECG tracings are kept as part of the participant's permanent study file at the site.

### Central Review

A centralized ECG laboratory is used in this study. Calibrated ECG machines are provided to sites and ECG traces are collected from the sites and data reviewed centrally and documented.

### Evaluation of Left Ventricular Ejection Fraction

Left ventricular ejection fraction should be evaluated at screening using ECHO or MUGA scan and this constitutes the baseline evaluation. A follow-up evaluation should be performed any time the participant shows signs or symptoms that may be associated with cardiac failure (such as shortness of breath, exercise intolerance, and peripheral edema) during the study. The evaluation of the left ventricular ejection fraction scheduled to occur during the safety follow-up should use the same modality as the baseline evaluation performed at screening.

### Ophthalmologic Examination

Real-world data has shown that BRAF inhibitors can cause ocular adverse effects (Mettler et al., Ocular safety profile of BRAF and MEK inhibitors: data from the World Health Organization Pharmacovigilance Database. Ophthalmology. 2021; 128: 1748-55). While currently there is no evidence suggesting that treatment with RAF dimer inhibitors as a class, or for Compound A specifically, i.e., based on the emerging data from the ongoing Compound A clinical study, may lead to an increased incidence of ocular AEs, it is recommended that full ophthalmologic assessment, including for visual acuity, intraocular pressure (provided as a numerical value), slit lamp examination, cup-to-disc ratio, dilated fundoscopy and optical coherence tomography (OCT), be undertaken as indicated for this study. Other methods may be performed if indicated by the investigator, an optometrist, or the ophthalmologist (e.g., fluorescein angiography, etc.). A full ophthalmic examination should be performed at screening, and while on study approximately every 8 (±1) weeks from the first dose of study drugs in the first 12 months and approximately every 12 (±1) weeks for the second year. If a participant remains on study beyond 2 years, exams may be performed approximately every 16 (±2) weeks. If participants report new visual disturbances such as diminished central vision, blurry vision, or loss of vision at time, a prompt ophthalmological assessment is required with follow-up as needed. Participants who wear contact lenses should be evaluated for any signs or symptoms of keratitis. Any CS findings and symptoms, including those confirmed by the ophthalmologist, must be reported as an AE.

Assessment of ECOG PS is required throughout the study at timepoints delineated in the SOA (see Table 7). Clinical laboratory safety assessments may be performed locally with results and local laboratory normal values entered into the eCRF. Collection of blood samples for laboratory assessments (serum chemistry, hematology, coagulation, and thyroid function) and urinalysis are performed at the timepoints indicated in the SOA (see Table 7) and samples are analyzed by the site local laboratory. If the safety laboratory screening tests are performed >96 hours prior to the first administration of study drug on Cycle 1 Day 1, then these tests should be repeated and reviewed within 48 hours before first study drug administration.

Blood samples for CPK monitoring are taken at screening; Cycle 1 Day 1, Day 8, and Day 15; on Day 1 of every subsequent treatment cycle from Cycle 2 onwards; and at the EoT visit and safety follow-up visits.

All participants should have their electrolytes monitored (for hypomagnesemia and hypocalcemia) during treatment with panitumumab and for 8 weeks after the completion of panitumumab therapy.

Blood samples for assessing coagulation parameters are collected at screening; at Cycle 1 Day 1; and then at Day 1 of every cycle only if the participant is taking anticoagulants, and as clinically indicated.

Thyroid function is assessed from blood samples collected at screening and on Day 1 of every 3 cycles after baseline, starting at Cycle 4 (Cycle 4, 7, 10 etc.). Local laboratory thyroid function testing is conducted based on the analysis of thyroid stimulating hormone (TSH), free T3, and free T4.

Additional blood collection for safety laboratory tests may be performed at other times if deemed necessary by the investigator, in conjunction with the Sponsor, i.e., for repeat laboratory or safety evaluations, including follow-up to AEs.

Participants are not required to fast prior to collection of blood samples for safety laboratory testing. Detailed instructions on the handling and processing of specimens are provided in the study laboratory manual.

### Serology

Hepatitis B surface antigen (HBsAg), antibodies against HBsAg, hepatitis core antibody (HBcAb), and HCV serology are tested at screening. In addition, viral load assessment (HBV DNA or HCV RNA) is done for participants with HBsAg positive or HCV antibody positive at screening, respectively.

In participants with known HIV, CD4+ T-cell counts should be performed at screening.

### Human Chorionic Gonadotropin (HCG) Pregnancy Test

A serum pregnancy test must be performed and documented as negative within 7 days prior to the first administration of the study drugs. A negative pregnancy test (serum or urine test) must be documented within 2 days prior to initiating treatment on Day 1 of each cycle thereafter. A serum pregnancy test is undertaken at the EoT visit (and in cases of premature discontinuation of study participation) and at the safety follow-up visit scheduled for 30+7 days after last dose of study drugs. If a urine pregnancy test is positive, it must be confirmed by a serum pregnancy test. Postmenopausal status is confirmed and documented through confirmation of FSH levels at screening for amenorrheic female participants, as applicable.

### Tumor Response Evaluation

Tumor response (antitumor efficacy) is assessed by either computed tomography (CT) or magnetic resonance imaging (MRI), with preference for CT (of chest with or without contrast and abdomen and pelvis with oral contrast, unless contraindicated). Positron emission tomography/CT (PET/CT) is permitted as an additional assessment if indicated by the investigator. The same imaging modality and radiographic procedure used to assess disease sites at screening is required to be used throughout the study (i.e., the same contrast protocol for scans).

Tumor imaging is performed within 28 days prior to first administration of study drug, and while on study approximately every 8 (±1) weeks from the first dose of study drugs in the first 12 months and approximately every 12 (±1) weeks for the second year. If a participant remains on study beyond 2 years, scans may be performed approximately every 16 (±2) weeks. In addition, participants must have a CT/MRI scan of the brain at screening to confirm presence or absence of CNS metastasis. Participants with CNS metastases at baseline should have follow-up CT/MRI of the brain following the scanning schedule.

Tumor response and progression of the cancer under study is assessed using RECIST v.1. The RECIST criteria takes priority regarding participant treatment and discontinuation decisions. Participants who are found to have clinical or radiological PD by RECIST v1.1 are discontinued from study treatment.

Participants who discontinue study treatment early for reasons other than RECIST-defined disease progression (e.g., toxicity) continue to undergo tumor assessments following the tumor response assessment schedule (see Table 7) until the participant begins a subsequent anticancer treatment, experiences disease progression, withdraws consent, dies, or until the study terminates, whichever occurs first.

### Central Review

Study imaging including CT and/or MRI should be performed per the schedule in the SOA (see Table 7) at a qualified imaging facility. For all participants in the dose expansion portion, sites submit all CT and/or MRI scans to the central imaging core laboratory for Central Imaging Review. The purpose of the Central Imaging Review is to provide an independent, unbiased, and objective review of the CT and MRI data. Sites are provided with an Imaging Acquisition Manual and an Imaging Submission Manual, which describe the imaging methods and submission process that must be followed. All image data submitted to the central imaging core laboratory must be de-identified prior to submission. The participant identifiers on the imaging data must be consistent with all study-related documents throughout the study. Study investigators can readily obtain details on de-identification requirements from available imaging manuals.

### Pharmacokinetic Assessments

Blood samples for plasma PK analysis of Compound A and any relevant metabolites (if appropriate) are obtained and processed according to instructions provided in the study laboratory manual at the time points delineated PK sampling table (see Table 7). The PK of panitumumab may be assessed in this study.

PK samples are collected at Cycle 1 Day 1 and Cycle 2 Day 1 for the quantification of Compound A and any relevant metabolites, if appropriate. On Day 1 of Cycle 1 and Cycle 2, blood samples for PK analysis are collected prior to Compound A dosing and at 2 to 4 hours post-Compound A dose. Starting from Cycle 3, blood samples for PK analysis are collected prior to dosing with Compound A on Day 1 of each treatment cycle for determination of steady state Ctrough.

The timing of PK sample collection may be altered and/or PK samples may be obtained at additional time points to ensure appropriate PK monitoring. The actual collection time of each sample must be recorded in the source data, on the collection tube and in the eCRF and provided to the bioanalytical laboratory.

The concentration of Compound A and any relevant metabolites (if appropriate) in plasma is determined by an accredited laboratory, using a suitably qualified and validated chromatography method.

Shipping, storage, and handling of samples for the PK assays is managed through a bioanalytical laboratory. Instruction manuals and supply kits are provided for all PK assessments.

### Pharmacodynamic Assessments

### Mutation Status for Study Eligibility

Collection of tumor tissue at the screening visit, either from archived tumor tissue or fresh tumor biopsies, is mandatory to establish baseline retrospective mutation status. Participants are selected for screening and eligibility based on known mutation status through the local molecular testing obtained from the tumor tissue sample collected any time prior to screening. Participants with CRC with a known mutation status and tumor harboring an oncogenic mutation of BRAF, KRAS, or NRAS are enrolled to Part 1 of the study. Participants with CRC with KRAS or NRAS mutations and participants with PDAC that harbors KRAS mutations are enrolled to Part 2 of the study.

Collection of fresh baseline tumor biopsies for analysis at screening is strongly recommended in participants with readily accessible tumor lesions. All study participants have mutational status determined locally using fresh or archival tissue, at any time prior to screening.

### Retrospective Confirmatory Mutation Analysis

The mutation result from the local molecular testing assay must be confirmed by a central analysis testing assay. Confirmatory mutation testing should be performed at the central laboratory at any time during the study to support the study data analysis. When possible, the tumor sample tested locally should be the same as the sample tested centrally.

Definitive central laboratory mutation results (positive or negative) cannot be repeated. If a sample is determined to be inadequate or the result is indeterminate on central testing, an additional sample may be resubmitted to the central laboratory for retesting. If there is non-concordance between the local and central laboratory results, or if the local result cannot be confirmed by central laboratory (e.g., inadequate sample or poor sample quality), participants may continue receiving study treatment provided there is no clinical deterioration or disease progression and the participant is benefiting from study treatment, as determined by the investigator and in consultation with the Sponsor medical monitor. In such cases, participants are informed as soon as possible that their mutation status is unconfirmed with information to describe follow-up procedures and alternative treatment options.

Tumor tissue should be of good quality based on total and viable tumor content. Fine-needle aspiration, brushing, cell pellets from pleural effusion, and lavage samples are not acceptable.

Fresh biopsies should be limited to readily accessible tumor lesions (i.e., skin, peripheral lymph nodes, lung, liver, or internal lymph node metastases which can be readily accessed using CT guidance). Acceptable fresh biopsy samples include core needle biopsies for deep tumor tissue or excisional, incisional, punch, or forceps biopsies for cutaneous, subcutaneous, or mucosal lesions. If performed, 3-5 tissue cylinders should be obtained that have proper size for histological examination and biomarker analysis. A minimum of 3 cores per biopsy is required.

Freshly collected tumor biopsies, when available, and archival tumor tissues (tissues should either be a formalin-fixed paraffin-embedded block or approximately 15 unstained slides) are sent to a central laboratory for mutation analysis. The panel of genes to be evaluated for baseline mutation testing for retrospective confirmatory analysis by the central laboratory is described in the laboratory manual.

### Blood Sampling for Analysis of Biomarkers

Peripheral whole blood samples are collected at timepoints as delineated in Table 7 for analysis of PDx biomarkers that include but are not limited to mutational, amplification, transcriptional, and/or phosphorylation alterations of MAPK pathway signaling. Shipping, storage, and handling of tumor tissues and blood samples for the assessment of biomarkers are managed through a central laboratory. The methods of sample handling and testing can readily be obtained from the common knowledge in the art.

Blood samples are also collected for analysis of the following tumor prognostic biomarkers in participant serum samples: Carcinoembryonic antigen (CEA) in all Part 1 dose finding cohorts and in Part 2 dose expansion Group 1 (participants with CRC that harbors KRAS or NRAS mutations); and Carbohydrate antigen 19-9 (CA19-9) in Part 2 dose expansion Group 2 only (participants with PDAC that harbors KRAS mutations).

All blood samples for analysis of tumor prognostic biomarkers are collected prior to dosing with the study drugs during the treatment period, and at other timepoints as indicated in Table 7. These blood samples are analyzed locally.

### Statistical Methods and Sample Size Determination

Statistical methods are outlined in a statistical analysis plan (SAP), which is finalized before the final database lock. The study results are presented by study part and dose level cohort/group (as applicable). The study results may be listed by participant as appropriate. In general, descriptive statistics (mean, standard deviation, median, minimum, and maximum) are calculated for summaries of continuous data, and frequency counts and percentages (where appropriate) are calculated for summaries of discrete/categorical data. If participants have missing or uninterpretable data for safety, efficacy, PK or PDx, or if a participant withdraws from the study during the DLT period for reasons other than toxicity, the investigator, in consultation with the Sponsor, may enroll an additional participant to replace the missing information and maintain the planned sample size for the analysis. Baseline is defined as the last non-missing evaluable measurement obtained prior to administration of study drugs on Day 1 of Cycle 1. No statistical hypotheses are formally evaluated in this study.

### Population

### Sample Size Rationale Dose Finding (Part 1):

The sample size for Part 1 consists of approximately 24 evaluable participants. Actual sample size is dependent on the number of dose escalation cohorts, as guided by the mTPI-2 model-assisted design.

### Dose Expansion (Part 2):

The initial goal of accrual for the dose expansion portion of the study is 20 evaluable participants in each of Group 1 (CRC patients with KRAS or NRAS mutations who have been treated and had documented disease progression by RECIST criteria during or after at least 1 line of prior therapy), and Group 2 (PDAC patients with KRAS mutations who have been treated and had documented disease progression by RECIST criteria during or after at least 1 line of prior therapy).

Dose expansion groups 1 and 2 are evaluated separately. If promising preliminary efficacy results are observed in either group after treating all planned participants (e.g., based on higher ORR or longer PFS), more participants may be added to the relevant group to further assess efficacy before progressing to Phase 2/3 clinical development.

### Populations for Analysis

The populations for analysis defined for the study are presented in Table 6.

Participant inclusion into each analysis population are determined after database lock and prior to unblinding for the final analysis.

TABLE 6
Populations for Analysis

| Population | Description |
|---|---|
| All Enrolled Population | All participants who sign an informed |
| | consent form and satisfy eligibility |
| | criteria. |
| Safety Population | All participants who receive at least 1 |
| | dose of the study drug/s (either |
| | Compound A or panitumumab). |
| Efficacy Evaluable Population | All dosed participants who have |
| | radiologically confirmed evaluable |
| | disease at baseline, and at least |
| | 1 evaluable post-baseline, radiological |
| | tumor response assessment. |
| Modified Intent-to-Treat (mITT) | All dosed participants who have |
| Population | radiologically confirmed evaluable |
| | disease at baseline. |
| Dose Limiting Toxicities (DLT) | Participants who receive ≥80% of the |
| Evaluable Population | assigned dose of Compound A in |
| | combination with both doses of |
| | panitumumab and remain on study for 28 |
| | days |
| | (4 weeks) from Cycle 1 Day 1 |
| | administration of Compound A + |
| | panitumumab or who experience a DLT |
| | preventing the participant from receiving |
| | 28 days of therapy during the defined |
| | dose finding window in Cycle 1. |
| Pharmacokinetic (PK) Population | All dosed participants with at least one |
| | quantifiable plasma sample of Compound |
| | A or relevant metabolites. |
| Pharmacodynamic (PDx) Population | All participants with valid Compound A + |
| | panitumumab PDx sampling after |
| | treatment with Compound A + |
| | panitumumab. |

### Statistical Analysis

### Safety Analysis

Safety for Parts 1 and 2 is determined by the reporting of AEs and by safety laboratory values (hematology, clinical chemistry, thyroid function testing, coagulation, and urinalysis). Vital signs, ECG and ECHO/MUGA scan (as applicable) findings, physical and ophthalmological examinations, and ECOG PS are also used to determine the safety profile of the Compound A+panitumumab combination. Safety endpoints are summarized using the Safety population.

The incidence of AEs is presented as the number (percentage) of participants with TEAEs by system organ class (SOC) and preferred term (PT) using the most current Medical Dictionary for Regulatory Activities (MedDRA) available at the time of study commencement, additionally the proportion of participants with dose interruptions or reductions of Compound A or panitumumab due to TEAEs is summarized. Descriptive summary statistics (i.e., n, mean, standard deviation, median, minimum, maximum for continuous variables; n [%] for categorical variables) and changes from baseline are determined for laboratory parameters, ECGs,

ECHO/MUGA scan (as applicable) and vital signs. Shifts from baseline in ECOG PS are summarized descriptively using frequency counts and percentages at the protocol scheduled time points. Newly occurring or worsening CS abnormalities identified on physical examination are captured as AEs and are not summarized or listed separately. For ophthalmological examinations, the overall assessment by the ophthalmologist (normal, abnormal not clinically significant (NCS), abnormal CS) is summarized. In case the result of the ophthalmologist's exam is abnormal in only one eye, then the result is summarized as abnormal for the overall eye exam. The results of the eye examination are listed by participant, and by eye, for each test method.

Additionally for Part 1, the MTD is determined in accordance with the mTPI-2 design and is based on the occurrence of DLTs in the first 28 days of Cycle 1. This analysis is conducted on the DLT evaluable population. At the end of Part 1, the MTD is selected as the dose with the smallest difference between the isotonically transformed posterior mean of the toxicity probability and target toxicity rate among the doses with posterior toxicity probability being greater than 0.30.

In the dose finding portion, participants are carefully monitored for AEs/SAEs. The recommendations according to the mTPI-2 design are followed, and if the lowest dose is under investigation and the maximum UPM is at an interval above (0.25, 0.33), then the trial is terminated due to excessive toxicity. Additionally, the study is terminated using Safety Rule 1 where if the posterior toxicity probability being greater than 0.30 is more than 95%, for the lowest dose, the trial is terminated due to excessive toxicity. Otherwise, the study is continued using the mTPI-2 recommendations and Safety Rule 2.

### Efficacy Analysis

The efficacy analysis is conducted on the mITT population for Parts 1 and 2. The efficacy endpoints based on response assessments using RECIST v1.1 (i.e., ORR, DOR, DCR, and PFS) are summarized to evaluate the antitumor activities of Compound A+panitumumab combination.

ORR is defined as the proportion of participants with confirmed CR or PR. DOR is defined as the time from the first determination of response in those participants with a confirmed response until the first documentation of progression or death due to any cause, whichever occurs first. DCR is defined as the proportion of participants with best overall response (BOR) of confirmed CR, PR, or SD ≥24 weeks. PFS is defined as the time from the date of the first administration of study drugs to the date of the first documentation of disease progression or death due to any cause, whichever occurs first.

The ORR and DCR is summarized with the 2-sided exact (Clopper-Pearson) 95% CI. The time-to-event endpoints, including PFS and DOR is analyzed by Kaplan-Meier methods. Statistical methods for analysis of efficacy are described in detail in the SAP.

The Efficacy Evaluable Population includes all dosed patients who have radiologically confirmed evaluable disease at baseline, and at least 1 evaluable post-baseline radiological tumor response assessment.

### Pharmacokinetics Analysis

PK blood samples are collected for the quantification of Compound A and any relevant metabolites, if appropriate, in plasma. Compound A (and any relevant metabolites) plasma concentration data may not be summarized but provided as a listing. Additional PK analyses including population PK (PopPK) analysis may be conducted as appropriate. These analyses may be reported separately from the CSR.

Exposure-response (efficacy or safety endpoints) analysis may be carried out if supported by data. Correlations between PK and biomarker endpoints may be explored as appropriate. Results of such analyses may be reported separately from the CSR.

### Pharmacodynamic and Other Exploratory Analysis

Summary statistics are provided for predictive and PDx biomarkers, including but not limited to assessment of the MAPK pathway for mutational, amplification, transcriptional, and/or phosphorylation status in the baseline tumor tissue and peripheral blood samples. Depending on the data available, the analysis for biomarkers may be descriptive in nature.

The results of exploratory analyses may be reported separately from the CSR.

A number of references have been cited, the disclosures of which were incorporated herein by reference in their entirety.

TABLE 7
Schedule of Assessments for the Study in Example 3 (Part 1 Dose Finding and Part 2 Dose Expansion)

| | Study Periods | | | | | | | | | | | |
|---|---|---|---|---|---|---|---|---|---|---|---|---|
| | | | | | | | | | | End of | | |
| | | | | | | | | | | Treatment | | |
| | | Treatment (28 days per cycle) | | | | | | | | (EoT)^{z} | Safety Follow-up | |
| | Screening^{a} | Cycle 1^{x} | | | | Cycle 2-8 | | ≥Cycle 9^{z} | | ≤7 days | 30 (+7) | 60 (+7) |
| | Day/s (window) | | | | | | | | | after study | days after | days after |
| | | | | | D 22 | | | | | drug | last dose | last dose |
| | D −28 to | | D 8 | D 15 | (±2 d) | D 1 | D 15 | D 1 | D 15 | discon- | of study | of |
| | D −1 | D 1 | (±1 d) | (±2 d) | TC^{y} | (+2 d) | (±2 d) | (±3 d) | (±2 d) | tinued | drugs | panitumumab |
| Informed consent^{b} | X | | | | | | | | | | | |
| Eligibility criteria | X | X^{e} | | | | | | | | | | |
| review | | | | | | | | | | | | |
| Demographics^{c} | X | | | | | | | | | | | |
| Medical history^{d} | X | X^{e} | | | | | | | | | | |
| Prior medications | X | X^{e} | | | | | | | | | | |
| Pregnancy test (for | X | X | | | | X | | X | | X | X | |
| WOCBP only) or | | | | | | | | | | | | |
| FSH test^{f} | | | | | | | | | | | | |
| Complete physical | X | | | | | | | | | X | X | X |
| exam^{g} | | | | | | | | | | | | |
| Limited physical | | X | | | | X | | X | | | | |
| exam^{g} | | | | | | | | | | | | |
| Ophthalmologic | X | Every 8 (±1) weeks from first dose in the first year. | | | | | | | | X | X | |
| exam^{h} | | Approximately every 12 (±1) weeks in the second year. | | | | | | | | | | |
| | | Approximately every 16 (±2) weeks >2 years. | | | | | | | | | | |
| Heightⁱ | X | | | | | | | | | | | |
| Weightⁱ | X | X | | | | X | | X | | X | X | |
| Vital signs^{j} | X | X | X | X | | X | X | X | X | X | X | |
| 12-lead ECG^{k} | X | X | X | | | X^{k} | | | | X | X | |
| ECHO/MUGA | X | As clinically indicated, based on participant symptoms | | | | | | | | | X | |
| scan^{l} | | | | | | | | | | | | |
| Viral serology^{m} | X | | | | | | | | | | | |
| Hematology, serum | X | X | X | X | | X | | X | | X | X | X |
| chemistry | | | | | | | | | | | | |
| (including CPK | | | | | | | | | | | | |
| testing)ⁿ | | | | | | | | | | | | |
| Coagulationⁿ | X | X | | | | D 1 of every cycle if the | | | | X | X | |
| | | | | | | participant is taking | | | | | | |
| | | | | | | anticoagulants, and as | | | | | | |
| | | | | | | clinically indicated | | | | | | |
| Thyroid function | X | | | | | D 1 of every 3 cycles after | | | | X | X | |
| testing^{o} | | | | | | baseline, starting at Cycle 4 | | | | | | |
| | | | | | | (Cycle 4, 7, 10 etc.) | | | | | | |
| Urinalysis^{p} | X | X | X | | | X | | X | | X | X | |
| ECOG PS^{q} | | X | | | | X | | X | | X | X | |
| Tumor evaluation | X | Every 8 (±1) weeks from first dose in the first year. | | | | | | | | X | | |
| (imaging/ | | Approximately every 12 (±1) weeks in the second year. | | | | | | | | | | |
| RECIST v1.1)^{r} | | Approximately every 16 (±2) weeks >2 years. | | | | | | | | | | |
| Tumor tissue | X | | | | | | | | | | | |
| biopsy (archival or | | | | | | | | | | | | |
| fresh)^{s} | | | | | | | | | | | | |
| Exploratory | | X | Every 8 (±1) weeks from first dose in the first year. | | | | | | | X | | |
| biomarker whole | | | Approximately every 12 (±1) weeks in the second year. | | | | | | | | | |
| blood sample | | | | | | | | | | | | |
| collection | | | | | | | | | | | | |
| Blood for CEA | | X | | | | X | | X | | X | X | |
| monitoring^{t} | | | | | | | | | | | | |
| Dose Finding Cohorts | | | | | | | | | | | | |
| and Dose Expansion | | | | | | | | | | | | |
| Group 1 only | | | | | | | | | | | | |
| Blood for CA19-9 | | X | | | | X | | X | | X | X | |
| monitoring^{t} | | | | | | | | | | | | |
| Dose Expansion | | | | | | | | | | | | |
| Group 2 only | | | | | | | | | | | | |
| Study drug | | | | | | | | | | | | |
| administration^{u} | | | | | | | | | | | | |
| Compound A | | X (continuous daily dosing) | | | | | | | | | | |
| (PO QD) | | | | | | | | | | | | |
| Panitumumab | | X | | X | | X | X | X | X | | | |
| (IV infusion Q2W) | | | | | | | | | | | | |
| Dispense study | | X | | | | X | | X | | | | |
| drug/re-supply | | | | | | | | | | | | |
| (Compound A) | | | | | | | | | | | | |
| Review returned | | | | | | X | | X | | X | | |
| study drug for | | | | | | | | | | | | |
| accountability and | | | | | | | | | | | | |
| compliance | | | | | | | | | | | | |
| (Compound A) | | | | | | | | | | | | |
| PK blood sampling^{v} | | X | | | | X | | X | | | | |
| AE review^{w} | X | X | X | X | X | X | X | X | X | X | X | |
| Concomitant | | X | X | X | X | X | X | X | X | X | X | |
| medication review^{w} | | | | | | | | | | | | |

Abbreviations: AE=adverse event; CA19-9=carbohydrate antigen 19-9; CEA=carcinoembryonic antigen; CPK=creatine phosphokinase; CRC=colorectal cancer; CT=computed tomography; D/d=day; DLT=dose limiting toxicity; ECG=electrocardiogram; ECHO=echocardiogram; ECOG PS=Eastern Cooperative Oncology Group Performance Status; EoT=end of treatment; FSH=follicle-stimulating hormone; IV=intravenous; MRI=magnetic resonance imaging; MUGA=multiple-gated acquisition; PDx=pharmacodynamic; PDAC=pancreatic ductal adenocarcinoma; PK=pharmacokinetics; PO=per oral; Q2W=once every 2 weeks; QD=once daily; RECIST v1.1=Response Evaluation Criteria in Solid Tumors version 1.1; TC=telephone contact; WOCBP=women of childbearing potential.

Screening: Individuals who do not meet the criteria for participation in this study (screen failure) for administrative reasons, or who have borderline test results, may be rescreened once. Rescreened participants should repeat all abnormal screening test and procedures.

Informed consent must be documented before any study-specific procedure, including for screening, is performed.

Demographics: Year of birth, age (calculated), sex, self-reported race/ethnicity is recorded as part of the screening procedures. Female participants are assessed as WOCBP or nonchildbearing potential.

Medical history: Includes any history of clinically significant (CS) disease, surgery, and cancer history.

Eligibility criteria, medical history and prior/ongoing medication use: Review prior to first administration of study drugs on Day 1 of Cycle 1 and document any changes since screening.

Pregnancy or FSH testing: Serum pregnancy test (for women of childbearing potential (WOCBP)) must be performed and documented as negative within 7 days prior to the first administration of the study drugs. A negative pregnancy test (serum or urine test) must be documented within 2 days prior to initiating treatment on Day 1 of each cycle thereafter. A serum pregnancy test is undertaken at the end of treatment (EoT) visit (and in cases of premature discontinuation of study participation) and at the safety follow-up visit (30+7 days after last dose of study drugs). If a urine pregnancy test is positive, it must be confirmed by a serum pregnancy test. Postmenopausal status is confirmed through testing of follicle-stimulating hormone (FSH) levels (>30 IU/L) at screening for amenorrheic female participants.

Physical examination: Complete and limited, symptom-directed physical examinations is performed by a licensed physician. A complete physical examination includes assessments of 1) head, eyes, ears, nose, throat 2) cardiovascular, 3) dermatological, 4) musculoskeletal, 5) respiratory, 6) gastrointestinal, and 7) neurological systems. Potential dermatological toxicity is assessed as part of the physical examination with the characteristics and grade of rash. Limited, symptom-directed examinations are performed at the specified time points or as clinically indicated. Physical examinations may be performed at various unscheduled time points if deemed necessary by the investigator.

Ophthalmologic examination: Full ophthalmic assessment, including for visual acuity, intraocular pressure (provided as a numerical value), slit lamp examination, cup-to-disc ratio, dilated fundoscopy, and optical coherence tomography (OCT), should be performed at screening, and while on study approximately every 8 (±1) weeks from the first dose of study drugs in the first 12 months and approximately every 12 (±1) weeks for the second year. If a participant remains on study beyond 2 years, exams may be performed approximately every 16 (±2) weeks. Other methods may be performed if indicated by the investigator, an optometrist, or the ophthalmologist (e.g., fluorescein angiography, etc.). If participants report new visual disturbances such as diminished central vision, blurry vision, or loss of vision at any time, a prompt ophthalmological assessment is required with follow-up as needed. For participants who use contact lenses, please ensure evaluations include careful review for keratitis. Any CS findings and symptoms, including those confirmed by the ophthalmologist, must be reported as an AE.

Height and weight: Height is measured only at screening. Body weight is monitored throughout the study and is to be recorded using the same scales (where possible) at each time point.

Vital signs include measurements of temperature (tympanic), heart rate, respiratory rate, and blood pressure (systolic and diastolic) after the participant has been sitting for at least 5 minutes. Pulse oximetry should also be performed and documented. At the specified visits, vital signs should be taken within 15 minutes pre-dose and within 15 minutes post-dose of each study drug administration.

ECG: A 12-lead ECG is performed at screening and during the treatment period within 60 minutes pre-dose and between 2 to 4 hours post-Compound A dose for Cycle 1 Day 1, Cycle 1 Day 8, and Cycle 2, Day 1. ECGs are required at EoT and in the safety follow-up period. ECGs beyond Cycle 2 Day 1 is not required but may be obtained if clinically indicated. To reduce spurious readings, every effort should be made to perform ECGs in triplicate, with 1- to 2-minute intervals between ECG readings; however, a single ECG is allowed, if necessary, i.e., due to a public health emergency. ECGs are performed with participants in a supine or semi-reclined position for at least 5 minutes before the reading is taken. All ECG tracings are reviewed by the investigator or a qualified designee. Heart rate, PR, QRS, QTcF, RR and result interpretation are recorded. Additional ECG monitoring may be performed at other times if deemed necessary by the investigator.

ECHO/MUGA: The same methodology should be used throughout the study, either consistently using ECHO or MUGA.

Viral serology: HBV and HCV tests include HBV and HCV serology (hepatitis B surface antigen (HBsAg), hepatitis B surface antibody (HBsAb), hepatitis B core antibody (HBcAb), and HCV antibody). In addition, viral load assessment (HBV deoxyribonucleic acid (DNA) or HCV ribonucleic acid (RNA)) are done for participants with HBsAg positive or HCV antibody positive at screening, respectively. In participants with known HIV, CD4+ T-cell counts should be performed at screening.

Hematology, chemistry, and coagulation: Local laboratory assessments for hematology, serum chemistry, and coagulation parameters are conducted. If the screening tests are performed >96 hours prior to the first administration of study drug on Cycle 1 Day 1, then the tests should be repeated and reviewed within 48 hours before first study drug administration. Local laboratory assessments may be performed up to 2 days prior to Day 1 (−2).

### In Addition:

Blood samples for creatine phosphokinase (CPK) monitoring are taken at screening; Cycle 1 Day 1, Day 8, and Day 15; on Day 1 of every subsequent treatment cycle from Cycle 2 onwards; and at the EoT visit (as applicable) and safety follow-up visits.
All participants should have their electrolytes monitored (for hypomagnesemia and hypocalcemia) during treatment with panitumumab and for 8 weeks after the completion of panitumumab therapy.
Participants are not required to fast prior to collection of blood samples for safety laboratory testing.

Thyroid function testing: Local laboratory thyroid function testing for thyroid stimulating hormone (TSH), free T3, and free T4.

Urinalysis: Local laboratory assessments for urinalysis parameters are conducted.

### Eastern Cooperative Oncology Group.

Tumor imaging—Either computed tomography (CT) or magnetic resonance imaging (MRI), with preference for CT of chest with or without contrast and abdomen and pelvis with oral contrast, is performed within 28 days prior to first administration of study drug and while on study approximately every 8 (±1) weeks from the first dose of study drugs in the first 12 months and approximately every 12 (±1) weeks for the second year. Positron emission tomography/CT (PET/CT) is permitted as an additional assessment if indicated by the investigator. Scans that are completed within the time window but prior to signing the ICF can be used for baseline scan. If a participant remains on study beyond 2 years, scans may be performed approximately every 16 (±2) weeks. The same imaging technique should be used in a participant throughout the study. In addition, participants must have a CT/MRI scan of the brain at screening to confirm presence or absence of CNS metastasis. Participants with CNS metastases at baseline should have follow-up CT/MRI of the brain following the scanning schedule.

Tumor tissue biopsy: Collection of tumor tissue at the screening visit, either from archived tumor tissue or fresh tumor biopsies, is mandatory to establish baseline retrospective mutation status. Collection of fresh baseline tumor biopsies for analysis at screening is strongly recommended in participants with readily accessible tumor lesions. Participants are selected for screening and eligibility based on known mutation status from the local molecular testing result obtained from the tumor tissue sample collected any time prior to screening. The mutation result from the local molecular testing is confirmed by testing undertaken at the central laboratory. The central testing is retrospective and may occur at any time during the study. Tumor samples should be submitted as either be a formalin-fixed paraffin-embedded block or approximately 15 unstained slides. Where possible, the tumor sample tested centrally should be the same as the locally tested sample.

Blood samples for monitoring of carcinoembryonic antigen (CEA) and carbohydrate antigen 19-9 (CA19-9): Blood samples are collected for analysis of CEA in dose finding cohorts and in dose expansion Group 1 (participants with colorectal cancer (CRC) that harbors KRAS or NRAS mutations). Blood samples are collected for analysis of CA19-9 in dose expansion Group 2 only (participants with pancreatic ductal adenocarcinoma (PDAC) that harbors KRAS mutations). All blood samples for analysis of tumor biomarkers are collected prior to dosing with the study drugs during the treatment period and these are analyzed locally.

Study drug administration: Compound A is administered orally (PO) once daily (QD). Panitumumab is administered once every 2 weeks (Q2W; on Day 1 and Day 15 of each treatment cycle) by intravenous (IV) infusion and is given within 60 minutes (+30 minutes) after oral dosing with Compound A. All participants undergo repeated Compound A+panitumumab treatment cycles of 28 days. Participants are administered the study drugs (Compound A followed by panitumumab) onsite at study visits conducted on Day 1 and Day 15 of each treatment cycle. Participants are instructed to self-administer daily doses of Compound A on all other study days in the treatment period (i.e., days 2-14 and days 16-28 per treatment cycle).

PK: Blood samples for analysis of Compound A and any relevant metabolites in plasma (if appropriate) are collected on Cycle 1 Day 1 and Cycle 2 Day 1 (pre-dose and 2 to 4 hours post-Compound A dose) and on Day 1 only of each subsequent cycle (prior to Compound A dosing on these days).

AEs and concomitant medication review: Elicitation of all AEs, as well as concomitant medication use occur at each interaction with the participant from the time of informed consent. Participants are also instructed to inform the investigator or clinic staff of any AEs or intercurrent illnesses experienced during the trial. For the purposes of tolerability decisions in Part 1, a DLT is defined for this study as any AE or abnormal laboratory value assessed as unrelated to underlying disease, disease progression, intercurrent illness or concomitant medications/therapies that occurs within the first 28-day cycle of treatment with the study drugs and meets at least 1 of the criteria

DLT period: The incidence of DLTs is evaluated during the first 28 days of treatment of Cycle 1 with the Compound A+panitumumab combination in Part 1 (dose finding portion).

Cycle 1 Day 22 Telephone Contact: Monitoring for AEs and concomitant medication use is conducted via follow-up telephone contact (TC) between site staff and the study participant on Day 22 of the first treatment cycle. If required, following the TC, an onsite visit is scheduled, at the investigator's discretion.

End of Treatment: All participants receive study drugs until clinical or radiological disease progression; cessation of study treatment due to death, intolerance, or withdrawal of consent from the study; completion of 2 years of treatment (unless the investigator's benefit-risk assessment supports continued treatment); investigator's decision; or the study is stopped by the Sponsor for any reason. All participants who discontinue study participation prematurely complete all specified assessments at an EoT visit, wherever possible. The EoT visit is optimally conducted within 7 days after the investigator determines that study drug is no longer to be used. Participants who discontinue study treatment early for reasons other than disease progression (e.g., toxicity) continue to undergo tumor assessments following the tumor response assessment schedule per protocol until the participant begins a subsequent anticancer treatment, experiences disease progression, withdraws consent, dies, or until the study terminates, whichever occurs first.

### Note:

Investigators or participants may request additional, unscheduled onsite visits at any time throughout the study. Assessments at unscheduled visits are undertaken as clinically indicated. Whenever vital signs, 12-lead ECGs, and PK/PDx biomarker blood draws are scheduled for the same nominal time, the assessments should occur in the following order: 12-lead ECG, vital signs, PK/PDx biomarker blood draws; so that the timing of the assessments allow the blood draw to occur at the exact nominal time.
Participants who discontinue treatment with the study drugs early for reasons other than disease progression (e.g., toxicity) continue to undergo tumor assessments following the tumor response assessment schedule per protocol until the participant begins a subsequent anticancer treatment, experiences disease progression, withdraws consent, dies, or until the study terminates, whichever occurs first.

A number of references have been cited, the disclosures of which were incorporated herein by reference in their entirety.

What is claimed is:

1. A combination comprising
(i) Compound A having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or the structure of formula (I): 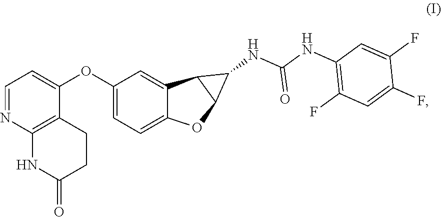 [SMILES: O=C1CCc2c(Oc3ccc4c(c3)[C@H]3[C@H](NC(=O)Nc5cc(F)c(F)cc5F)[C@H]3O4)ccnc2N1]
or a pharmaceutically acceptable salt, tautomer, stereoisomer, enantiomer, isotopologue, solvate, or prodrug thereof; and
(ii) an anti-EGFR antibody or an antigen binding fragment thereof.

2. The combination of claim 1, wherein the combination comprises an anti-EGFR antibody.

3. The combination of claim 2, wherein the anti-EGFR antibody is
(a) panitumumab; or
(b) an isolated human antibody comprising a heavy chain immunoglobulin molecule comprising:
a) a CDR1 comprising amino acids 8 to 15 of SEQ ID NO: 3;
b) a CDR2 comprising amino acids 29 to 45 of SEQ ID NO: 3; and
c) a CDR3 comprising amino acids 77 to 85 of SEQ ID NO: 3;
and comprising a light chain immunoglobulin molecule comprising:
d) a CDR1 comprising amino acids 5 to 15 of SEQ ID NO: 4;
e) a CDR2 comprising amino acids 31 to 37 of SEQ ID NO: 4; and
f) a CDR3 comprising amino acids 70 to 78 of SEQ ID NO: 4.

4. The combination of claim 2, wherein Compound A is in the form of Form F.

5. A combination kit comprising a combination of any one of claims 1-4 together with a pharmaceutically acceptable carrier or carriers.

6. The combination kit of claim 5, wherein the anti-EGFR antibody or an antigen binding fragment thereof is provided in a form suitable for IV administration.

7. The combination kit of claim 5, wherein the an anti-EGFR antibody or an antigen binding fragment thereof is provided in a form suitable for subcutaneous administration.

8. A method of treating a cancer in a subject in need thereof, comprising administering to the subject the combination or combination kit of any one of claims 1-7.

9. A method of treating a cancer in a subject in need thereof, comprising administering to the subject simultaneously
(i) Compound A having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or the structure of formula (I): 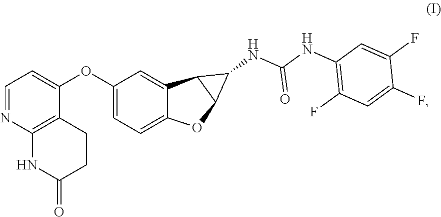 [SMILES: O=C1CCc2c(Oc3ccc4c(c3)[C@H]3[C@H](NC(=O)Nc5cc(F)c(F)cc5F)[C@H]3O4)ccnc2N1]
or a pharmaceutically acceptable salt, tautomer, stereoisomer, enantiomer, isotopologue, solvate, or prodrug thereof; and
(ii) an anti-EGFR antibody.

10. A method of treating a cancer in a subject in need thereof, comprising administering to the subject
Compound A having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or the structure of formula (I): 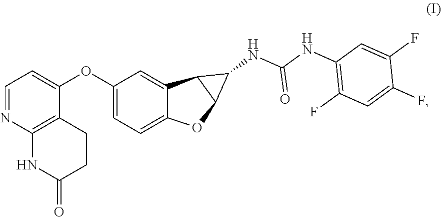 [SMILES: O=C1CCc2c(Oc3ccc4c(c3)[C@H]3[C@H](NC(=O)Nc5cc(F)c(F)cc5F)[C@H]3O4)ccnc2N1]
or a pharmaceutically acceptable salt, tautomer, stereoisomer, enantiomer, isotopologue, solvate, or prodrug thereof,
wherein the subject has received an anti-EGFR antibody.

11. A method of treating a cancer in a subject in need thereof, comprising administering to the subject an anti-EGFR antibody,
wherein the subject has received Compound A having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or the structure of formula (I): 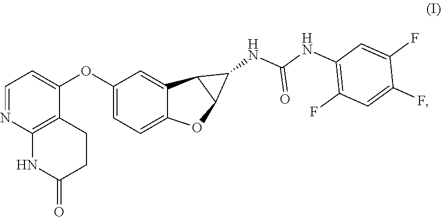 [SMILES: O=C1CCc2c(Oc3ccc4c(c3)[C@H]3[C@H](NC(=O)Nc5cc(F)c(F)cc5F)[C@H]3O4)ccnc2N1]
or a pharmaceutically acceptable salt, tautomer, stereoisomer, enantiomer, isotopologue, solvate, or prodrug thereof.

12. The combination or combination kit of any one of claims 1-7 for use in treating a cancer in a subject in need thereof.

13. Use of Compound A having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or the structure of formula (I): 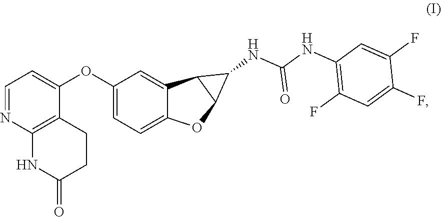 [SMILES: O=C1CCc2c(Oc3ccc4c(c3)[C@H]3[C@H](NC(=O)Nc5cc(F)c(F)cc5F)[C@H]3O4)ccnc2N1]
or a pharmaceutically acceptable salt, tautomer, stereoisomer, enantiomer, isotopologue, solvate, or prodrug thereof in the manufacture of a medicament for treating a cancer in a subject in need thereof, where the medicament is suitable for being administered with panitumumab.

14. Use of panitumumab in the manufacture of a medicament for treating a cancer in a subject in need thereof, where the medicament is suitable for being administered with Compound A having the name of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or the structure of formula (I): 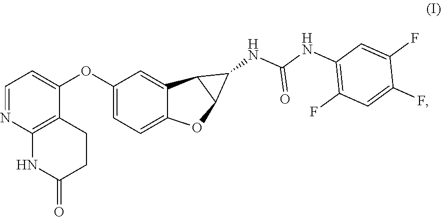 [SMILES: O=C1CCc2c(Oc3ccc4c(c3)[C@H]3[C@H](NC(=O)Nc5cc(F)c(F)cc5F)[C@H]3O4)ccnc2N1]
or a pharmaceutically acceptable salt, tautomer, stereoisomer, enantiomer, isotopologue, solvate, or prodrug thereof.

15. Use of the combination of any one of claims 1-4 in the manufacture of a medicament for treating a cancer in a subject in need thereof.

16. The method, combination, combination kit, or use of any one of claims 8-15, wherein the cancer is colorectal cancer, pancreatic cancer, or non-small cell lung cancer.

17. The method, combination, combination kit, or use of claim 16, wherein the cancer is colorectal cancer, metastatic colorectal cancer, BRAF mutant metastatic colorectal cancer, BRAF V600E mutant metastatic colorectal cancer, KRAS mutant colorectal cancer, KRAS G12C mutant colorectal cancer, KRAS G12D mutant colorectal cancer, KRAS G12V mutant colorectal cancer, NRAS mutant colorectal cancer, or TRP53 mutant colorectal cancer.

18. The method, combination, combination kit, or use of any one of claims 8-15, wherein the cancer is pancreatic cancer, pancreatic ductal adenocarcinoma (PDAC), BRAF mutant pancreatic cancer, BRAF V600E mutant pancreatic cancer, KRAS mutant pancreatic cancer, KRAS G12C mutant pancreatic cancer, KRAS G12D mutant pancreatic cancer, KRAS G12V mutant pancreatic cancer, Trp53 mutant pancreatic cancer, or NRAS mutant pancreatic cancer.

19. The method, combination, combination kit, or use of any one of claims 8-15, wherein the cancer is non-small cell lung cancer, BRAF mutant non-small cell lung cancer, BRAF V600E mutant non-small cell lung cancer, KRAS mutant non-small cell lung cancer, KRAS G12C mutant non-small cell lung cancer, KRAS G12D mutant non-small cell lung cancer, KRAS G12V mutant non-small cell lung cancer, Trp53 mutant non-small cell lung cancer, or NRAS mutant non-small cell lung cancer.

20. The method, combination, combination kit, or use of any one of claims 8-15, wherein the cancer is colorectal cancer, pancreatic cancer, melanoma, non-small cell lung cancer, brain cancer, lung cancer, kidney cancer, bone cancer, liver cancer, bladder cancer, breast cancer, head and neck cancer, ovarian cancer, skin cancer, adrenal cancer, cervical cancer, lymphoma, or thyroid cancer.

21. The method, combination, combination kit, or use of any one of claims 8-15, wherein the cancer is characterized by a mutation in a gene selected from RAS, NRAS, KRAS, RAF, BRAF, CRAF, ARAF, and any combination thereof.

22. The method, combination, combination kit, or use of claim 18, wherein the cancer is characterized by a mutation in a gene selected from RAS, NRAS, KRAS, RAF, BRAF, and any combination thereof.

23. The method, combination, combination kit, or use of claim 22, wherein the cancer is characterized by a mutation in a gene selected from NRAS, KRAS, BRAF, and any combination thereof.

24. The method, combination, combination kit, or use of any one of claims 8-15, wherein the cancer is characterized by:
(i) a mutation in a gene selected from ARAF, BRAF, RAF1, KRAS, HRAS, NF1, MAP2K1, MAP2K2, MAPK1, and any combination thereof,
(ii) a mutation selected from BRAF N20T, BRAF A33T, BRAF S36A, BRAF V47_G393del, BRAF V47_G327del, BRAF V47_D380del, BRAF V47_M438del, BRAF N49I, BRAF M53I, BRAF L64I, BRAF G69S, BRAF A81_D380del, BRAF A81_M438del, BRAF G104E, BRAF T119S, BRAF P141L, BRAF S151A, BRAF P162S, BRAF V169_G327del, BRAF V169_D380del, BRAF R188T, BRAF Q201H, BRAF G203_G393del, BRAF K205Q, BRAF V226L, BRAF E228V, BRAF R239Q, BRAF T241P, BRAF T241M, BRAF L245F, BRAF A246P, BRAF F247L, BRAF Q2579 BRAF Q257H, BRAF G258V, BRAF F259L, BRAF Q2629 BRAF H269Y, BRAF R271H, BRAF E275K, BRAF D287H, BRAF F294L, BRAF T310I, BRAF A320T, BRAF I326V, BRAF P341S, BRAF R347*, BRAF P348T, BRAF S363F, BRAF S364L, BRAF P367S, BRAF P367R BRAF P367L, BRAF D380H, BRAF R389C, BRAF T401I, BRAF A404Cfs*9, BRAF P407L, BRAF S419Y, BRAF G421V, BRAF R444W, BRAF D448Y, BRAF D449Y, BRAF W450*, BRAF W450L, BRAF E451K, BRAF E451Q, BRAF P453T, BRAF V459L, BRAF R462E, BRAF R462K, BRAF R462I, BRAF 1463T, BRAF 1463S, BRAF G4641, BRAF G4649 BRAF G464E, BRAF G464A, BRAF G464V, BRAF S465D, BRAF S465E, BRAF S465A, BRAF G466R BRAF G466E, BRAF G466A, BRAF G466V, BRAF S467A, BRAF S467L, BRAF F468C, BRAF G469L, BRAF G469del, BRAF G469S, BRAF G469R BRAF G469E, BRAF G469A, BRAF G469V, BRAF T470K, BRAF V471I, BRAF V471F, BRAF Y472dup, BRAF Y472S, BRAF Y472C, BRAF G478C, BRAF K483E, BRAF K483M, BRAF L485_P490del, BRAF L485Y, BRAF L485_P490delinsY, BRAF L485S, BRAF L485W, BRAF L485F, BRAF L485_P490delinsF, BRAF N486_Q494del, BRAF N486del, BRAF N486_T488del, BRAF N486_T491del, BRAF N486_L495del, BRAF N486D, BRAF N486_V487del, BRAF N486 P490del, BRAF N486_A489delinsK, BRAF N486_T491delinsK, BRAF V487_P490del, BRAF V487_P492delinsA, BRAF T488_P492del, BRAF T488_Q493delinsK, BRAF A489_P490del, BRAF P490del, BRAF P490_Q494del, BRAF K499E, BRAF K499N, BRAF E501K, BRAF E501G, BRAF V504 R506dup, BRAF V504I, BRAF L505F, BRAF L505H, BRAF R509G, BRAF R509H, BRAF L514V, BRAF M5171, BRAF Q524L, BRAF L525R BRAF T529M, BRAF T529N, BRAF T529I, BRAF W531C, BRAF G534D, BRAF Y538H, BRAF R558Q, BRAF G563D, BRAF H568D, BRAF H574N, BRAF H574Y, BRAF H574Q, BRAF N581D, BRAF N581Y, BRAF N581T, BRAF N581S, BRAF N581I, BRAF N581K, BRAF I582M, BRAF F583C, BRAF L584F, BRAF H585Y, BRAF E586K, BRAF D587A, BRAF D587G, BRAF D587E, BRAF V590I, BRAF V590G, BRAF 1592V, BRAF 1592M, BRAF G593D, BRAF D594N, BRAF D594H, BRAF D594Y, BRAF D594_T599dup, BRAF D594A, BRAF D594G, BRAF D594V, BRAF D594E, BRAF F595L, BRAF F595S, BRAF G596S, BRAF G5969 BRAF G596C, BRAF G596D, BRAF G596V, BRAF L597S, BRAF L597V, BRAF L597Q, BRAF L597P, BRAF L597R, BRAF A598T, BRAF A598S, BRAF A598V, BRAF A598_T599insARC, BRAF A598_T599insV, BRAF T599dup, BRAF T599A, BRAF T599K, BRAF T599R BRAF T599I, BRAF T599_V600insTT, BRAF T599_V600insS, BRAF T599_V600insETT, BRAF T599_V600insEAT, BRAF V600_K601delinsEN, BRAF V600_S605delinsEISRWR, BRAF V600K, BRAF V6009 BRAF V600Q, BRAF V600dup, BRAF V600delinsYM, BRAF V600M, BRAF V600L, BRAF V600D, BRAF V600_K601delinsE, BRAF V600E, BRAF V600A, BRAF V600G, BRAF K601del, BRAF K601Q, BRAF K601E, BRAF K601_W604del, BRAF K601T, BRAF K601I, BRAF K601_S602delinsNT, BRAF K601N, BRAF S602T, BRAF S602Y, BRAF S602F, BRAF R603*, BRAF W604del, BRAF W604R BRAF W604G, BRAF S605A, BRAF S605F, BRAF S605E, BRAF S605G, BRAF S605N, BRAF S605I, BRAF G606W, BRAF G606E, BRAF G606A, BRAF G606V, BRAF S607P, BRAF S607F, BRAF H6089 BRAF Q609E, BRAF Q609L, BRAF Q609H, BRAF E61D, BRAF L613F, BRAF G615J BRAF L618F, BRAF W6191 BRAF S637*, BRAF V639I, BRAF E648Q, BRAF Y656D, BRAF R671Q, BRAF P676S, BRAF L678I, BRAF V681I, BRAF E695K, BRAF K698R, BRAF L711F, BRAF A712T, BRAF R719S, BRAF H725Y, BRAF A728V, BRAF P731T, BRAF P731S, BRAF P731L, BRAF A762E, BRAF A762V, and any combination thereof,
(iii) a mutation selected from KIAA1549-BRAF fusion, BCAS1-BRAF fusion, CCDC6-BRAF fusion, CDC42BPB-BRAF fusion, FAM131B-BRAF fusion, FXR1-BRAF fusion, GIT2-BRAF fusion, KLHL7-BRAF fusion, RNF130-BRAF fusion, TMEM106B-BRAF fusion, MKRN1-BRAF fusion, AGAP3-BRAF fusion, AGK-BRAF fusion, AKAP9-BRAF fusion, ARMC10-BRAF fusion, CUL1-BRAF fusion, GTF2I-BRAF fusion, PAPSS1-BRAF fusion, PCBP2-BRAF fusion, PPFIBP2-BRAF fusion, SND1-BRAF fusion, TRIM24-BRAF fusion, ZKSCAN1-BRAF fusion, SEPT3-BRAF fusion, and any combination thereof, or
(iv) a mutation selected from NRAS G12A, NRAS G12C, NRAS G12D, NRAS G12N, NRAS G12P, NRAS G12R NRAS G12S, NRAS G12V, NRAS G12Y, NRAS G3A, NRAS G13C, NRAS G13D, NRAS G3E, NRAS G13N, NRAS G13R NRAS G3S, NRAS G13V, NRAS A18T, NRAS I24N, NRAS P34L, NRAS Y40*, NRAS Q43*, NRAS T50I, NRAS T58I, NRAS A59G, NRAS A59D, NRAS A59T, NRAS G60E, NRAS G60R NRAS Q61E, NRAS Q61H, NRAS Q61H, NRAS Q61K, NRAS Q61L, NRAS Q61L, NRAS Q61P, NRAS Q61R NRAS Q61J NRAS Q61R NRAS Q61*, NRAS E63K, NRAS Y64D, NRAS S65C, NRAS R68S, NRAS S89A, NRAS G115Efs*46, NRAS E132K, NRAS K135N, NRAS A146P, NRAS A146T, NRAS A146V, NRAS E162*, and any combination thereof.

25. The method, combination, combination kit, or use of any one of claims 8-15, wherein the cancer is characterized by a mutation selected from NRAS Q61R, NRAS Q61K, NRAS Q61L, NRAS G12S, NRAS G13J KRAS G12A, KRAS G12C, KRAS G12D, KRAS G12V, BRAF V600E, BRAF fusion, and any combination thereof.

26. The method, combination, combination kit, or use of claim 25, wherein the cancer is characterized by a mutation selected from NRAS Q61R, NRAS Q61K, NRAS Q61L, KRAS G12D, KRAS G12V, BRAF V600E, BRAF fusion, and any combination thereof.

27. The method, combination, combination kit, or use of claim 26, wherein the cancer is characterized by a mutation selected from NRAS Q61R, NRAS Q61K, NRAS Q61L, KRAS G12D, KRAS G12V, and any combination thereof.

28. The method of any one of claims 8-11, wherein the cancer is characterized by MAPK pathway genomic aberration.

29. The method of any one of claims 8-11, wherein Compound A or a pharmaceutically acceptable salt, tautomer, stereoisomer, enantiomer, isotopologue, solvate, or prodrug thereof is administered one to three times a day.

30. The method of any one of claims 8-11, wherein Compound A or a pharmaceutically acceptable salt, tautomer, stereoisomer, enantiomer, isotopologue, solvate, or prodrug thereof is administered once a day.

31. The method of any one of claims 8-11, wherein Compound A or a pharmaceutically acceptable salt, tautomer, stereoisomer, enantiomer, isotopologue, solvate, or prodrug thereof is administered in an amount of from about 5 mg to about 60 mg per day.

32. The method of claim 31, wherein Compound A or a pharmaceutically acceptable salt, tautomer, stereoisomer, enantiomer, isotopologue, solvate, or prodrug thereof is administered at about 5 mg, about 10 mg, about 15 mg, about 20 mg, about 25 mg, about 30 mg, about 35 mg, about 40 mg, about 45 mg, about 50 mg, about 55 mg, or about 60 mg per day.

33. The method of any one of claims 8-11, wherein the method provides a plasma Compound A AUC₈ₕ between about 2,128 ng*h/ml and about 45,000 ng*h/ml in the subject.

34. The method of any one of claims 8-11, wherein panitumumab is administered in an amount of about 6 mg/kg.

35. The method of claim 34, wherein panitumumab is administered in an amount of about 6 mg/kg administered as an intravenous infusion over 60 minutes.

36. The method of claim 35, wherein panitumumab is administered in an amount of about 6 mg/kg administered as an intravenous infusion over about 60 minutes every two weeks.

37. The method of any one of claims 34 to 36, wherein panitumumab is coadministered with Compound A or a pharmaceutically acceptable salt or solvate thereof.

38. The method of any one of claims 8-11, wherein the subject achieves a stable disease, a partial response, or a complete response.

39. The method, combination, combination kit, or use of any one of claims 8-38, wherein panitumumab is administered intravenously in an amount of about 6 mg/kg every 14 days as an intravenous infusion over about 60 minutes, when the amount of panitumumab administered in 14 days is no more than about 1000 mg, or about 90 minutes, when the amount of panitumumab administered in 14 days is more than about 1000 mg; and Compound A is administered orally once per day at about 5 mg, about 10 mg, about 15 mg, about 20 mg, about 25 mg, about 30 mg, about 35 mg, or about 40 mg.
